(19) United States
(12) Patent Application Publication
(10) Pub. No.: US 2026/0137674 A1
(43) Pub. Date: May 21, 2026

(54) COMBINATION THERAPY USING A KRAS G12D INHIBITOR, AND A NUCLEOSIDE ANALOG, AND/OR A MICROTUBULE INHIBITOR

(71) Applicant: Incyte Corporation, Wilmington, DE (US)

(72) Inventors: Matthew Farren, Wilmington, DE (US); Valerie Roman, Wilmington, DE (US); Renee Wallower, Wilmington, DE (US)

(21) Appl. No.: 19/366,209

(22) Filed: Oct. 22, 2025

Related U.S. Application Data
(60) Provisional application No. 63/710,999, filed on Oct. 23, 2024.
(60) Provisional application No. 63/902,197, filed on Oct. 20, 2025.

(51) Int. Cl. A61K 31/4745; A61K 31/337; A61K 31/7068; A61K 47/64; A61P 35/00

(52) U.S. Cl. CPC A61K 31/4745; A61K 31/337; A61K 31/7068; A61K 47/643; A61P 35/00

(57) ABSTRACT

Provided herein are methods of treating cancer by administering a combination therapy comprising a KRAS G12D inhibitor, a nucleoside analog, and a microtubule inhibitor.

### RELATED APPLICATIONS

This application claims priority to U.S. Provisional Application No. 63/710,999 filed on Oct. 23, 2024, and U.S. Provisional Application No. 63/902,197 filed on Oct. 20, 2025, the content of each of which is incorporated in its entirety.

### BACKGROUND

KRAS mutations are among the most common genetic alterations in cancer (D. A. Erlanson et al., Curr. Opin. Chem. Biol., 2021, 62, 101-108). KRAS is a membrane-bound GTPase that, when activated through upstream receptor tyrosine kinases, promotes cell survival and proliferation (D. Uprety et al., Cancer Treat. Rev., 2020, 89, 102070). KRAS proteins exist in a GTP-bound “on” state and GDP bound “off” state. When GTP-bound, signals are transduced through activation of the mitogen activated protein kinase pathway and the PI3K pathway, in addition to others. KRAS mutations are found in approximately 23% of solid tumors. The G12D isoform is the most common, accounting for approximately 29% of KRAS mutations in cancer (J. K. Lee, et al., NPJ Precis. Oncol., 2022, 6, 91). KRAS G12D mutations are found in approximately 40% of pancreatic cancers (pancreatic ductal adenocarcinoma), 15% of colorectal carcinomas, and 5% of non-small cell lung adenocarcinomas, representing major unmet medical needs. The KRAS G12D mutation impairs GTP hydrolysis, resulting in a hyperactivated KRAS isoform that drives high levels of oncogenic ERK and PI3K signaling (M. Malumbres, et al., Nat. Rev. Cancer, 2003, 3(6), 459-65).

Inhibiting KRAS G12D is hypothesized to abrogate KRAS signaling and halt tumor growth in KRAS G12D mutant tumors. Inhibiting KRAS G12D in this way is hypothesized to abrogate KRAS signaling and halt tumor growth in KRAS G12D mutant tumors.

Taxanes are a class of microtubule inhibitors or stabilizers. This class includes paclitaxel (Taxol) and docetaxel (Taxotere), which are important drugs used in the treatment of cancer. Microtubule stabilizers and formulations of these agents continue to provide advances in cancer therapy.

Nucleoside analogs are structural analogs of a nucleoside, which normally contain a nucleobase and a sugar. While typically used as therapeutic antiviral products to prevent viral replication in infected cells, nucleoside analogs have also shown anti-cancer activity.

Current treatments for metastatic or advanced pancreatic cancer include chemotherapy combinations, such as gemcitabine and nab-paclitaxel (K. Ducreux, et al. Ann. Oncol., 2015, 26(Supp.5), v56-68),

### SUMMARY

Provided herein is a method of treating cancer in a subject in need thereof comprising administering to the subject a KRAS G12D inhibitor, or a pharmaceutically acceptable salt thereof, a nucleoside analog, or a pharmaceutically acceptable salt thereof, and a microtubule inhibitor, or pharmaceutically acceptable salt thereof. Targeting KRAS G12D mutant tumors with a selective and reversible inhibitor in combination with a nucleoside analog and/or a microtubule inhibitor can be a promising cancer treatment for patients with KRAS G12D mutations.

### BRIEF DESCRIPTION OF THE DRAWINGS

FIG. 1 shows antitumor activity of Compound 1±gemcitabine and nab-paclitaxel in the 2838c (KPCY-013) Model.

FIG. 2 shows antitumor activity of Compound 1±gemcitabine and nab-paclitaxel in HPAC Xenograft Tumors.

### DETAILED DESCRIPTION

Ras proteins are part of the family of small GTPases that are activated by growth factors and various extracellular stimuli. The Ras family regulates intracellular signaling pathways responsible for growth, migration, survival and differentiation of cells. Activation of Ras proteins at the cell membrane results in the binding of key effectors and initiation of a cascade of intracellular signaling pathways within the cell, including the RAF and PI3K kinase pathways. Somatic mutations in RAS may result in uncontrolled cell growth and malignant transformation while the activation of RAS proteins is tightly regulated in normal cells (D. Simanshu, et al., Cell, 2017, 170(1), 17-33). The Ras family is comprised of three members: KRAS, NRAS and HRAS. RAS mutant cancers account for about 25% of human cancers. KRAS is the most frequently mutated isoform accounting for 85% of all RAS mutations whereas NRAS and HRAS are found mutated in 12% and 3% of all Ras mutant cancers respectively (D. Simanshu, et al., Cell, 2017, 170(1), 17-33). KRAS mutations are prevalent amongst the top three most deadly cancer types: pancreatic (97%), colorectal (44%), and lung (30%) (A. D. Cox, et al. Nat. Rev. Drug. Discov., 2014, 13(11), 828-51). The majority of RAS mutations occur at amino acid residue 12, 13, and 61. The frequency of specific mutations varies between RAS gene isoforms and while G12 and Q61 mutations are predominant in KRAS and NRAS respectively, G12, G13 and Q61 mutations are most frequent in HRAS. Furthermore, the spectrum of mutations in a RAS isoform differs between cancer types. HRAS. Furthermore, the spectrum of mutations in a RAS isoform differs between cancer types. For example, KRAS G12D mutations predominate in pancreatic cancers (40%), followed by colorectal adenocarcinomas (15%) and lung cancers (5%)(Lee J K, et al. NPJ Precis. Oncol., 2022, 6, 459-465). Genomic studies across hundreds of cancer cell lines have demonstrated that cancer cells harboring KRAS mutations are highly dependent on KRAS function for cell growth and survival (R. McDonald, et al., Cell, 2017, 170(3), 577-92). The role of mutant KRAS as an oncogenic driver is further supported by extensive in vivo experimental evidence showing mutant KRAS is required for early tumor onset and maintenance in animal models (A. D. Cox, et al. Nat. Rev. Drug. Discov., 2014, 13(11), 828-51).

The present disclosure is related to methods of treating cancer in a subject in need thereof comprising administering to the subject a KRAS G12D inhibitor, or a pharmaceutically acceptable salt thereof, a nucleoside analog, or a pharmaceutically acceptable salt thereof, and/or a microtubule inhibitor, or pharmaceutically acceptable salt thereof.

Certain terms used herein are described below. Compounds of the present disclosure are described using standard nomenclature. Unless defined otherwise, all technical and scientific terms used herein have the same meaning as is commonly understood by one of skill in the art to which this invention belongs.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art.

Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

The term “about” when used in connection with a numerical value, means that a collection or range of values is included. For example, “about X” includes a range of values that are ±10%, ±5%, ±2%, ±1%, ±0.5%, ±0.2%, or ±0.1% of X, where X is a numerical value.

In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

As used herein, “pharmaceutical combination” or “combination” refers to formulations of the separate compounds with or without instructions for combined use or to combination products. The combination compounds may thus be entirely separate pharmaceutical dosage forms or in pharmaceutical compositions that are also sold independently of each other and where instructions for their combined use are provided in the package equipment, e.g., leaflet or the like, or in other information, e.g., provided to physicians and medical staff (e.g., oral communications, communications in writing or the like), for simultaneous or sequential use for being jointly active.

The term “treat,” “treated,” “treating,” or “treatment” includes the diminishment or alleviation of at least one symptom associated or caused by the state, disorder or disease being treated. In certain embodiments, the treatment comprises bringing into contact with KRAS an effective amount of a compound disclosed herein for conditions related to cancer.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the term “free base equivalent” refers to the amount of active agent, or a pharmaceutically acceptable salt of the active agent (e.g., Compound 1) that is equivalent to the free-base of the active agent dose. Stated alternatively, the term “free base equivalent” means either an amount of Compound 1 free base, or the equivalent amount of Compound 1 free base that is provided by a pharmaceutically acceptable salt of said compound.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein a parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts described herein include the conventional non-toxic salts of the parent compound formed, for example, from non-toxic inorganic or organic acids. The pharmaceutically acceptable salts discussed herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, EtOAc, ethanol, isopropanol, or MeCN are used. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66(1), 1-19, S. Gaisford in A. Adejare (Ed.), Remington, The Science and Practice of Pharmacy, 23^{rd} Ed., (Elsevier, 2020), Chapter 17, pp. 307-14; S. M. Berge et al., J. Pharm. Sci., 1977, 66(1), 1-19, T. S. Wiedmann, et al., Asian J. Pharm. Sci., 2016; 11, 722-34. D. Gupta et al., Molecules, 2018, 23(7), 1719; P. H. Stahl et al., Handbook of Pharmaceutical Salts: Properties, Selection, and Use, (Wiley, 2002) and in P. H. Stahl et al., Handbook of Pharmaceutical Salts: Properties, Selection, and Use, 2^{nd} Ed. (Wiley, 2011).

As used herein, the term “composition” or “pharmaceutical composition” refers to a mixture of at least one compound with a pharmaceutically acceptable carrier. The pharmaceutical composition facilitates administration of the composition to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound disclosed herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of a compound disclosed herein, and are physiologically acceptable to the patient. Supplementary active compounds may also be incorporated into the compositions. The “pharmaceutically acceptable carrier” may further include a pharmaceutically acceptable salt of the compound(s) disclosed herein. Other additional ingredients that may be included in the pharmaceutical compositions are known in the art and described, for example, in P. Beringer, et al., (Eds.), Remington: The Science and Practice of Pharmacy, 21^{st} Ed.; (Lippincott Williams & Wilkins: Philadelphia, Pa., 2005); A. Adejare (Ed.), Remington, The Science and Practice of Pharmacy, 23^{rd} Ed., (Elsevier, 2020); R. C. Rowe et al., Eds., Handbook of Pharmaceutical Excipients, 6^{th} Ed.; (Pharmaceutical Press, 2009); P. J. Shesky et al., Eds., Handbook of Pharmaceutical Excipients, 9^{th} Ed.; (The Pharmaceutical Press, 2020); M. Ash, et al., (Eds.), Handbook of Pharmaceutical Additives, 3^{rd} Ed.; (Gower Publishing Company: 2007); and M. Gibson (Ed.), Pharmaceutical Preformulation and Formulation, 2^{nd} Ed. (CRC Press LLC, 2009).

The term “single formulation” as used herein refers to a single carrier or vehicle formulated to deliver effective amounts of both therapeutic agents to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

The term “combination therapy” refers to the administration of two or more therapeutic compounds to treat a therapeutic condition or disorder described in the present disclosure. Such administration encompasses co-administration of these therapeutic compounds in a substantially simultaneous manner, such as in a single capsule having a fixed ratio of active ingredients or in multiple, or in separate containers (e.g., capsules) for each active ingredient. In addition, such administration also encompasses use of each type of therapeutic compound in a sequential manner, either at approximately the same time or at different times. In either case, the treatment regimen will provide beneficial effects of the drug combination in treating the conditions or disorders described herein.

The combination of agents described herein may display a synergistic effect. The term “synergistic effect” as used herein, refers to action of two agents such as, for example, a KRAS inhibitor (e.g., a KRAS inhibitor of formula I) and a nucleoside analog in combination with a microtubule inhibitor, producing an effect, for example, slowing the symptomatic progression of cancer or symptoms thereof, which is greater than the simple addition of the effects of each drug administered by themselves. A synergistic effect can be calculated, e.g., using suitable methods such as the Sigmoid-Emax equation (N. H. G. Holford, et al., Clin. Pharmacokinet., 1981, 6: 429-53), the equation of Loewe additivity (S. Loewe, et al., Arch. Exp. Pathol Pharmacol., 1926, 114, 313-26) the median-effect equation (T. C. Chou, et al., Adv. Enzyme Regul., 1984, 22: 27-55), or based on the Bliss definition of drugs independence (E. Demidenko, et al., PLoS ONE, 2019, 14(11): e0224137). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

As used herein, the term “synergy” refers to the effect achieved when the active ingredients, i.e., KRAS inhibitor and a nucleoside analog in combination with a microtubule inhibitor, used together is greater than the sum of the effects that results from using the compounds separately.

As used herein, “nucleoside analog” refers to a drug that is a structural analog of a nucleoside, which normally contains a nucleobase (adenine, cytosine, guanine, thymine, uracil, and derivatives thereof) and a sugar. Non-limiting examples of a nucleoside analog include remdesivir, cytarabine, gemcitabine, entecavir, ribavirin, and aciclovir.

As used herein, “microtubule inhibitor” refers to an active agent that inhibits mitosis (cell division). These agents disrupt microtubules, which are structures that pull the chromosomes apart when a cell divides. Microtubule inhibitors fall into two categories: stabilizing agents and destabilizing agents. Stabilizing agents, i.e., microtubule stabilizers, include taxanes and epothilones. Microtubule stabilizers promote polymerization whereas destabilizing agents, e.g., vinca alkaloids, lead to depolymerization.

As used herein, “taxane” refers to a diterpine having the following taxadiene core:

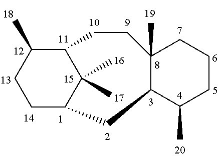

Examples of taxane include, but are not limited to, paclitaxel, docetaxel, cabazitaxel, nab-paclitaxel, or alternative formulations thereof.

In an embodiment, provided herein is a combination therapy comprising an effective amount of a KRAS inhibitor, a nucleoside analog, and a microtubule inhibitor. An “effective amount” of a combination of agents (i.e., a KRAS inhibitor, a nucleoside analog, and a microtubule inhibitor) is an amount sufficient to provide an observable improvement over the baseline clinically observable signs and symptoms of the disorders treated with the combination.

Provided herein is a combination of therapeutic agents and administration of the combination of agents to treat cancer, and related indications. As used herein, the term “cancer” includes related indications, such as anemia. As used herein, a “combination of agents” and similar terms refer to a combination of different types of agents: a KRAS G12D inhibitor, or a pharmaceutically acceptable salt thereof, a nucleoside analog, or a pharmaceutically acceptable salt thereof, and/or a microtubule inhibitor, or pharmaceutically acceptable salt thereof. Use of racemic mixtures of the individual agents is also provided.

Pharmacologically active metabolites include those that are inactive but converted into pharmacologically active forms in the body after administration.

As used herein, the term “alkyl,” by itself or as part of another substituent means, unless otherwise stated, a straight or branched chain hydrocarbon having the number of carbon atoms designated (i.e., C₁-C₆-alkyl means an alkyl having one to six carbon atoms) and includes straight and branched chains. In an embodiment, C₁-C₃, C₁-C₄, C₁-C₆ alkyl groups are provided herein. Examples include methyl, ethyl, propyl, isopropyl, butyl, isobutyl, tert-butyl, pentyl, neopentyl, and hexyl.

The term “alkylene,” employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

As used herein, the term “alkoxy,” refers to the group —O-alkyl, wherein alkyl is as defined herein. Alkoxy includes, by way of example, methoxy, ethoxy, n-propoxy, isopropoxy, n-butoxy, sec-butoxy, t-butoxy and the like. In an embodiment, C₁-C₃, C₁-C₄, C₁-C₆ alkoxy groups are provided herein.

The term “amino,” employed alone or in combination with other terms, refers to a group of formula —NH₂, wherein the hydrogen atoms may be substituted with a substituent described herein. For example, “alkylamino” can refer to —NH(alkyl) and —N(alkyl)₂.

As used herein, the term “halo” or “halogen” alone or as part of another substituent means, unless otherwise stated, a fluorine, chlorine, bromine, or iodine atom.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1}halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CC₃, CHCl₂, C₂Cl₅ and the like. In some embodiments, the haloalkyl group is a fluoroalkyl group.

The term “haloalkoxy,” employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

As used herein, the term “cycloalkyl” means a non-aromatic carbocyclic system that is partially or fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-10, 3-8, 3-7, 3-6, and 5-10 atoms. The term “cycloalkyl” includes, but is not limited to, cyclopropyl, cyclobutyl, cyclopentyl, cyclohexyl, bicyclo[3.1.0]hexyl, spiro[3.3]heptanyl, bicyclo[2.2.2]octanyl and bicyclo[1.1.1]pentyl. In an embodiment, 3-10 membered cycloalkyl groups are provided herein.

As used herein, the term “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. Heterocycloalkyl also includes bicyclic structures that may be bridged or spirocyclic in nature with each individual ring within the bicycle varying from 3-8, 5-10, 4-6, or 3-10 atoms, and containing 0, 1, or 2 N, O, or S atoms. The term “heterocycloalkyl” includes cyclic esters (i.e., lactones) and cyclic amides (i.e., lactams) and also specifically includes, but is not limited to, epoxidyl, oxetanyl, THFyl, tetrahydropyranyl (i.e., oxanyl), pyranyl, dioxanyl, aziridinyl, azetidinyl, pyrrolidinyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-aza-bicyclo[2.1.1]hexanyl, 5-azabicyclo[2.1.1]hexanyl, 6-azabicyclo[3.1.1]heptanyl, 2-azabicyclo-[2.2.1]heptanyl, 3-aza-bicyclo[3.1.1]heptanyl, 2-azabicyclo[3.1.1]heptanyl, 3-azabicyclo-[3.1.0]hexanyl, 2-aza-bicyclo[3.1.0]hexanyl, 3-azabicyclo[3.2.1]octanyl, 8-azabicyclo[3.2.1]-octanyl, 3-oxa-7-aza-bicyclo[3.3.1]nonanyl, 3-oxa-9-azabicyclo[3.3.1]nonanyl, 2-oxa-5-aza-bicyclo[2.2.1]heptanyl, 6-oxa-3-azabicyclo[3.1.1]heptanyl, 2-azaspiro[3.3]heptanyl, 2-oxa-6-azaspiro[3.3]heptanyl, 2-oxaspiro[3.3]heptanyl, 2-oxaspiro[3.5]nonanyl, 3-oxaspiro[5.3]-nonanyl, and 8-oxabicyclo-[3.2.1]octanyl. In an embodiment, 3-10 membered heterocycloalkyl groups are provided herein. In another embodiment, 5-10 membered heterocycloalkyl groups are provided herein. In still another embodiment, 4-6 membered heterocycloalkyl groups are provided herein.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “COₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments, aryl groups have 6 carbon atoms.

In some embodiments, aryl groups have 10 carbon atoms. In some embodiments, the aryl group is phenyl. In some embodiments, the aryl group is naphthyl.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thiophenyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]-pyridinyl, pyrazolo[1,5-a]pyridinyl, 5,6,7,8-tetrahydroisoquinolinyl, 5,6,7,8-tetra-hydroquinolinyl, 6,7-dihydro-5H-cyclopenta[b]pyridinyl, 6,7-dihydro-5H-cyclopenta[c]-pyridinyl, 1,4,5,6-tetrahydrocyclopenta[c]pyrazolyl, 2,4,5,6-tetrahydrocyclopenta[c]-pyrazolyl, 5,6-dihydro-4H-pyrrolo[1,2-b]pyrazolyl, 6,7-dihydro-5H-pyrrolo[1,2-b]-[1,2,4]triazolyl, 5,6,7,8-tetrahydro-[1,2,4]triazolo[1,5-a]pyridinyl, 4,5,6,7-tetrahydro-pyrazolo[1,5-a]pyridinyl, 4,5,6,7-tetrahydro-1H-indazolyl and 4,5,6,7-tetrahydro-2H-indazolyl. In an embodiment, 5-10 membered heteroaryl groups are provided herein.

It is to be understood that if a cycloalkyl, heterocycloalkyl, aryl, or heteroaryl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thioenyl, and so forth.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

### KRAS G12D Inhibitors

The present disclosure relates to a combination therapy comprising a KRAS G12D inhibitor, or a pharmaceutically acceptable salt thereof, in combination with a nucleoside analog, or a pharmaceutically acceptable salt thereof, and/or a microtubule inhibitor, or pharmaceutically acceptable salt thereof. This combination therapy can be used to treat various disorders associated with abnormal activity of KRAS.

In an embodiment, the KRAS G12D inhibitor is a compound of Formula I:

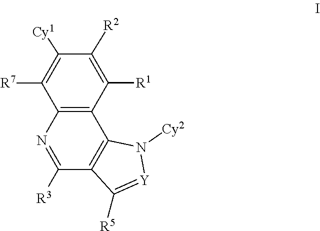

or a pharmaceutically acceptable salt thereof, wherein:
Y is N or CR⁶;
R¹ is selected from H, C₁₋₃ alkyl, C₁₋₃ haloalkyl, cyclopropyl, halo, D, CN, and OR^{a1}; wherein said C₁₋₃ alkyl and cyclopropyl are each optionally substituted with 1 or 2 substituents independently selected from R^{g};
R² is selected from H, C₁₋₃ alkyl, C₁₋₃ haloalkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene, halo, D, CN, and OR^{a2}; wherein said C₁₋₃ alkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene are each optionally substituted with 1 or 2 substituents independently selected from R⁹;
Cy¹ is selected from C₃₋₁₀ cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3, or 4 substituents independently selected from R¹⁰;
R³ is selected from H, C₁₋₃ alkyl, C₁₋₃ haloalkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR³, C(O)NR^{c3}R^{d3}, NR^{c3}R^{d3}, and NR^{c3}C(O)R^{b3}; wherein said C₁₋₃ alkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 substituents independently selected from R³⁰;
R⁵ is selected from H, C₁₋₃ alkyl, C₁₋₃ haloalkyl, cyclopropyl, halo, D, CN, and OR^{a5}; wherein said C₁₋₃ alkyl and cyclopropyl are each optionally substituted with 1 or 2 substituents independently selected from R⁹;
R⁶ is selected from H, C₁₋₃ alkyl, C₁₋₃ haloalkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR^{a6}, and C(O)NR^{c6}R^{d6}; wherein said C₁₋₃ alkyl, C₃₋₆cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1 or 2 substituents independently selected from R⁶⁰;
R⁷ is selected from H, C₁₋₃ alkyl, C₁₋₃ haloalkyl, cyclopropyl, halo, D, CN, and OR^{a7}; wherein said C₁₋₃ alkyl and cyclopropyl are each optionally substituted with 1 or 2 substituents independently selected from R^{g};
Cy² is selected from

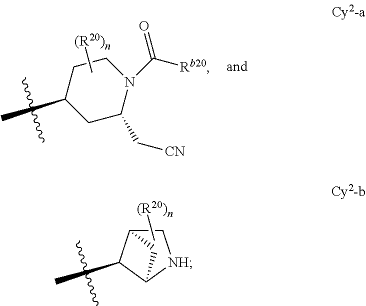

wherein n is 0, 1, or 2;
each R¹⁰ is independently selected from C₁₋₃ alkyl, C₁₋₃ haloalkyl, halo, D, CN, OR^{a10}, C(O)R^{b10}, C(O)NR^{c10}R^{d10}, C(O)OR^{a10}, NR^{c10}R^{d10}, and S(O)₂R^{b10};
each R²⁰ is independently selected from C₁₋₃ alkyl, C₁₋₃ haloalkyl, halo, D, CN, and OR^{a20};
each R³⁰ is independently selected from C₁₋₃ alkyl, C₁₋₃ haloalkyl, C₃₋₆ cycloalkyl, 4-6 membered heterocycloalkyl, phenyl, 5-6 membered heteroaryl, halo, D, CN, OR^{a30}, C(O)R^{b30}, C(O)NR^{c30}R^{d30}, C(O)OR^{a30}, NR^{c30}R^{d30}, and S(O)₂R^{b30}; wherein said C₁₋₃ alkyl, C₃₋₆ cycloalkyl, 4-6 membered heterocycloalkyl, phenyl, and 5-6 membered heteroaryl are each optionally substituted with 1 or 2 substituents independently selected from R³¹ each R³¹ is independently selected from C₁₋₃ alkyl, C₁₋₃ haloalkyl, halo, D, CN, OR^{a31}, C(O)R^{b31}, C(O)NR^{c31}R^{d31}, C(O)OR^{a3}1, NR^{c31}R^{d31}, and S(O)₂R^{b31};
each R³³ is independently selected from C₁₋₃ alkyl, C₁₋₃ haloalkyl, C₃₋₆ cycloalkyl, 4-membered heterocycloalkyl, 6-membered heterocycloalkyl, phenyl, 5-6 membered heteroaryl, halo, D, CN, OR^{a30}, C(O)NR^{c30}R^{d30}, and NR^{c30}R^{d30}; wherein said C₁₋₃ alkyl, C₃₋₆ cycloalkyl, 4-membered heterocycloalkyl, 6-membered heterocycloalkyl, phenyl, and 5-6 membered heteroaryl are each optionally substituted with 1 or 2 substituents independently selected from R³¹;
each R⁶⁰ is independently selected from C₁₋₃ alkyl, C₁₋₃ haloalkyl, 4-6 membered 20 heterocycloalkyl, 5-6 membered heteroaryl, halo, D, CN, OR^{a60}, C(O)R^{b60}, C(O)NR⁶⁰R^{d60} NR^{c60}C(O)R^{b60}, C(O)OR^{a60}, NR^{c60}C(O)OR^{a60}, NR^{c60}R^{d60}, NR^{c60}S(O)₂R^{b60}, and S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, C₁₋₃ haloalkyl, halo, D, CN, OR^{a61}, and NR^{c61}R^{d61};
R^{a1} is selected from H, C₁₋₃ alkyl, and C₁₋₃ haloalkyl;
each R^{a2} is independently selected from H, C₁₋₃ alkyl, and C₁₋₃ haloalkyl;
each R^{b3}, R^{c3} and R^{d3} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R³⁰;
or R^{c3} and R^{d3} attached to the same N atom, together with the N atom to which they are attached, form a 4-, 5-, or 6-membered heterocycloalkyl group optionally substituted with 1, 2, or 3 substituents independently selected from R³⁰
R^{j3} is selected from C₁₋₃ alkyl, C₁₋₃ haloalkyl, C₃₋₆ cycloalkyl, 4-6 membered heterocycloalkyl, phenyl, and 5-6 membered heteroaryl; wherein said C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R³⁰;
or R^{c3} and R^{j3} attached to the same N atom, together with the N atom to which they are attached, form a 4-, 5-, or 6-membered heterocycloalkyl group optionally substituted with 1, 2, or 3 substituents independently selected from R³⁰;
R^{f3} is selected from C₁₋₃ haloalkyl, C₃₋₆ cycloalkyl, 4-6 membered heterocycloalkyl, phenyl and 5-6 membered heteroaryl; wherein said C₁₋₃ haloalkyl, C₃₋₆ cycloalkyl, 4-6 membered heterocycloalkyl, phenyl, and 5-6 membered heteroaryl are each optionally substituted with 1, 2, or 3 substituents independently selected from R³⁰; or
R^{f3} is selected from

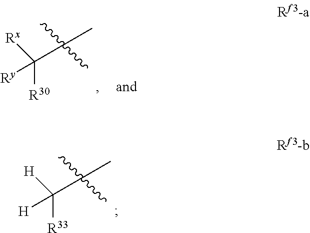

wherein R^{x} is H or C₁₋₂ alkyl and Ry is C₁₋₂ alkyl;
or R^{x} and R^{y}, together with the C atom to which they are attached, form a 3-, or 4-membered cycloalkyl group;
R^{a5} is selected from H, C₁₋₃ alkyl, and C₁₋₃ haloalkyl;
each R^{a6}, R^{c6} and R^{d6} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl and 5-6 membered heteroaryl are each optionally substituted with 1 or 2 substituents independently selected from R⁶⁰;
R^{a7} is selected from H, C₁₋₃ alkyl, and C₁₋₃ haloalkyl;
each R^{a10}, R^{b10}, R^{c10} and R^{d10} is independently selected from H, C₁₋₃ alkyl, and C₁₋₃ haloalkyl;
each R^{a20} is independently selected from H, C₁₋₃ alkyl, and C₁₋₃ haloalkyl;
R^{b20} is selected from NH₂, C₁₋₃ alkyl, and C₁₋₃ haloalkyl;
each R^{a30}, R^{b30}, R^{c30} and R^{d30}, is independently selected from H, C₁₋₃ alkyl, and C₁₋₃ haloalkyl;
each R^{a31}, R^{b31}, R^{c31} and R^{d31} is independently selected from H, C₁₋₃ alkyl, and C₁₋₃ haloalkyl;
each R^{a60}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆ cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹;
each R^{a61}, R^{c61}, and R^{d61}, is independently selected from H, C₁₋₃ alkyl, and C₁₋₃ haloalkyl; and
each R⁹ is independently selected from D, OH, CN, halo, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino.

In an embodiment of Formula I, or a pharmaceutically acceptable salt thereof,
Y is CR⁶;
R¹ is selected from H, C₁₋₃ alkyl, and C₁₋₃ haloalkyl;
R² is selected from H, C₁₋₃ alkyl, C₁₋₃ haloalkyl, halo, D, CN, and OR^{a2}; wherein said C₁₋₃ alkyl is optionally substituted with 1 or 2 substituents independently selected from R^{g};
Cy¹ is selected from C₃₋₁₀ cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3, or 4 substituents independently selected from R¹⁰;
R³ is selected from H, C₁₋₃ alkyl, C₁₋₃ haloalkyl, C₃₋₆ cycloalkyl, 4-6 membered heterocycloalkyl, phenyl, 5-6 membered heteroaryl, halo, D, CN, C(O)NR^{c3}R^{d3}, and NR^{c3}C(O)R^{b3}; wherein said C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R³⁰;
R⁵ is selected from H, C₁₋₃ alkyl, C₁₋₃ haloalkyl, and halo;
R⁶ is selected from H, C₁₋₃ haloalkyl, C₃₋₆ cycloalkyl, 4-8 membered heterocycloalkyl, phenyl, 5-6 membered heteroaryl, halo, D, CN, OR^{a6}, and C(O)NR^{c6}R^{d6}; wherein said C₃₋₆ cycloalkyl, 4-8 membered heterocycloalkyl, phenyl, and 5-6 membered heteroaryl are each optionally substituted with 1 or 2 substituents independently selected from R⁶⁰;
R⁷ is selected from H, C₁₋₃ alkyl, C₁₋₃ haloalkyl, halo, and CN;
Cy² is selected from

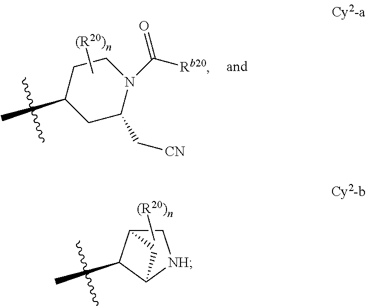

wherein n is 0, 1, or 2;
each R¹⁰ is independently selected from C₁₋₃ alkyl, C₁₋₃ haloalkyl, halo, D, CN, OR^{a10}, C(O)R^{b10}, C(O)NR^{c10}R^{d10}, C(O)OR^{a10}, NR^{c10}R^{d10}, and S(O)₂R^{b10};
each R²⁰ is independently selected from C₁₋₃ alkyl, C₁₋₃ haloalkyl, halo, D, CN, and OR^{a20};
each R³⁰ is independently selected from C₁₋₃ alkyl, C₁₋₃ haloalkyl, C₃₋₆ cycloalkyl, 4-6 membered heterocycloalkyl, phenyl, 5-6 membered heteroaryl, halo, D, CN, OR^{a30}, C(O)R^{b30}, C(O)NR^{c30}R^{d30}, C(O)OR^{a30}, NR^{c30}R^{d30}, and S(O)₂R^{b30}; wherein said C₁₋₃ alkyl, C₃₋₆ cycloalkyl, 4-6 membered heterocycloalkyl, phenyl, and 5-6 membered heteroaryl are each optionally substituted with 1 or 2 substituents independently selected from R³¹;
each R³¹ is independently selected from C₁₋₃ alkyl, C₁₋₃ haloalkyl, halo, D, CN, OR^{a31}, C(O)R^{b31}, C(O)NR^{c31}R^{d31}, C(O)OR^{a3}1, NR^{c31}R^{d31}, and S(O)₂R^{b31};
each R⁶⁰ is independently selected from C₁₋₃ alkyl, C₁₋₃ haloalkyl, C₁₋₃ haloalkoxy, 4-6 membered heterocycloalkyl, 5-6 membered heteroaryl, halo, D, CN, OR^{a60}, C(O)R^{b60}, C(O)NR^{c60}R^{d60}, NR^{c60}C(O)R^{b60}, C(O)OR^{a60}, NR^{c60}C(O)OR^{a60}, NR^{c60}R^{d60}, NR^{c60}S(O)₂R^{b60}, and S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, C₁₋₃ haloalkyl, halo, D, CN, OR^{a61}, and NR^{c61}R^{d61};
each R^{a2} is independently selected from H, C₁₋₃ alkyl, and C₁₋₃ haloalkyl;
each R^{b3}, R^{c3} and R^{d3} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said, C₁₋₃ alkyl, C₃₋₆ cycloalkyl, 4-6 membered heterocycloalkyl, phenyl and 5-6 membered heteroaryl are each optionally substituted with 1, 2, or 3 substituents independently selected from R³⁰;
or R^{c3} and R^{d3} attached to the same N atom, together with the N atom to which they are attached, form a 4-, 5-, or 6-membered heterocycloalkyl group optionally substituted with 1, 2, or 3 substituents independently selected from R³⁰;
each R^{a6}, R^{c6} and R^{d6} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl and 5-6 membered heteroaryl are each optionally substituted with 1 or 2 substituents independently selected from R⁶⁰;
each R^{a10}, R^{b10}, R^{c10} and R^{d10} is independently selected from H, C₁₋₃ alkyl, and C₁₋₃ haloalkyl;
each R^{a20} is independently selected from H, C₁₋₃ alkyl, and C₁₋₃ haloalkyl;
R^{b20} is selected from NH₂, C₁₋₃ alkyl, and C₁₋₃ haloalkyl;
each R^{a30}, R^{b30}, R^{c30} and R^{d30}, is independently selected from H, C₁₋₃ alkyl, and C₁₋₃ haloalkyl;
each R^{a31}, R^{b31}, R^{c31} and R^{d31} is independently selected from H, C₁₋₃ alkyl, and C₁₋₃ haloalkyl;
each R^{a6}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆ cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹; and
each R^{a61}, R^{c61}, and R^{d61}, is independently selected from H, C₁₋₃ alkyl, and C₁₋₃ haloalkyl; and
each R⁹ is independently selected from D, CN, halo, C₁₋₃ alkyl, and C₁₋₃ haloalkyl.

In another embodiment of Formula I, or a pharmaceutically acceptable salt thereof,
Y is CR⁶;
R¹ is H;
R² is selected from C₁₋₃ alkyl, C₁₋₃ haloalkyl, halo, CN, and —CH₂CH₂CN;
Cy¹ is selected from C₃₋₁₀ cycloalkyl, C₆₋₁₀ aryl and 6-10 membered heteroaryl; wherein the 6-10 membered heteroaryl has at least one ring-forming carbon atom and 1, ring-forming heteroatoms independently selected from N and S; and wherein the C₃₋₁₀ cycloalkyl, C₆₋₁₀ aryl and 6-10 membered heteroaryl are each optionally substituted with 1 or 2 substituents independently selected from R¹⁰;
R³ is selected from H, C₁₋₃ alkyl, C₁₋₃ haloalkyl, 4-6 membered heterocycloalkyl, phenyl, and 5-6 membered heteroaryl; wherein said C₁₋₃ alkyl, 4-6 membered heterocycloalkyl, phenyl, and 5-6 membered heteroaryl are each optionally substituted with 1, 2, or 3 substituents independently selected from R³⁰;
R⁵ is selected from H and halo;
R⁶ is selected from H, C₁₋₃ haloalkyl, 4-8 membered heterocycloalkyl, and 5-6 membered heteroaryl; wherein said 4-8 membered heterocycloalkyl and 5-6 membered heteroaryl are each optionally substituted with 1 or 2 substituents independently selected from R⁶⁰; or
R⁷ is halo;
Cy² is

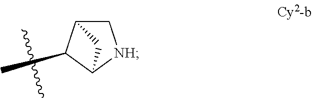

each R¹⁰ is independently selected from C₁₋₃ alkyl, C₁₋₃ haloalkyl, halo, D, CN, and OR^{a10};
each R³⁰ is independently selected from C₁₋₃ alkyl, C₁₋₃ haloalkyl, 4-6 membered heterocycloalkyl, halo, D, CN, OR^{a30}, C(O)NR^{c30}R^{d30}, and NR^{c30}R^{d30}; wherein said C₁₋₃ alkyl and 4-6 membered heterocycloalkyl are each optionally substituted with 1 or 2 substituents independently selected from R³¹;
each R³¹ is independently selected from C₁₋₃ alkyl, C₁₋₃ haloalkyl, halo, CN, OR^{a31}, and NR^{c31}R^{d31};
each R⁶⁰ is independently selected from C₁₋₃ alkyl, C₁₋₃ haloalkyl, C₁₋₃ haloalkoxy, 4-6 membered heterocycloalkyl, 5-6 membered heteroaryl, halo, D, CN, OR^{a60}, C(O)R^{b60}, C(O)NR^{c60}R^{d60}, NR^{c60}C(O)R^{b60}, C(O)OR^{a60}, NR^{c60}C(O)OR^{a60}, NR^{c60}R^{d60}, NR^{c60}S(O)₂R^{b60}, and S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, C₁₋₃ haloalkyl, halo, and CN;
each R^{a10} is independently selected from H and C₁₋₃ alkyl;
each R^{a30}, R^{c30} and R^{d30} is independently selected from H and C₁₋₃ alkyl;
each R^{a31}, R^{c31} and R^{d31} is independently selected from H and C₁₋₃ alkyl;
each R^{a6}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆ cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹.

In yet another embodiment of Formula I, or a pharmaceutically acceptable salt thereof,
Y is CR⁶;
R¹ is H;
R² is —CH₂CH₂CN;
Cy¹ is phenyl; wherein the phenyl is optionally substituted with 1 or 2 substituents independently selected from R¹⁰;
R³ is selected from H, C₁₋₃ alkyl, phenyl, and 5-6 membered heteroaryl; wherein said C₁₋₃ alkyl, phenyl, and 5-6 membered heteroaryl are each optionally substituted with 1, 2 or 3 substituents independently selected from R³⁰;
R⁵ is selected from H and halo;
R⁶ is selected from 4-8 membered heterocycloalkyl; wherein said 4-8 membered heterocycloalkyl is optionally substituted with 1 or 2 substituents independently selected from R⁶⁰; or
R⁶ is selected from C₁₋₃ alkyl; wherein said C₁₋₃ alkyl is substituted with 1 or 2 substituents independently selected from R⁶⁰;
R⁷ is halo;
Cy² is

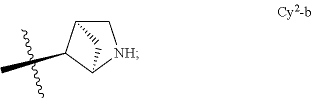

each R¹⁰ is independently selected from C₁₋₃ alkyl and halo;
each R³⁰ is independently selected from C₁₋₃ alkyl, halo, D, OH, and C(O)NR^{c30}R^{d30}; wherein said C₁₋₃ alkyl is optionally substituted with 1 substituent independently selected from R³¹;
each R³¹ is OR^{a31};
each R⁶⁰ is independently selected from C₁₋₃ alkyl, C₁₋₃ haloalkoxy, 4-6 membered heterocycloalkyl, 5-6 membered heteroaryl, halo, C(O)R^{b60}, C(O)NR^{c60}R^{d60}, NR^{c60}C(O)R^{b60}, C(O)OR^{a60}, NR^{c60}C(O)OR^{a60}, and NR^{c60}S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, and halo;
each R^{c30} and R^{d30} is independently selected from H and C₁₋₃ alkyl;
each R^{a31} is independently selected from H and C₁₋₃ alkyl; and
each R^{a60}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆ cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹.

In still another embodiment of Formula I, or a pharmaceutically acceptable salt thereof,
Y is CR⁶;
R¹ is H;
R² is —CH₂CH₂CN;
Cy¹ is phenyl; wherein the phenyl is optionally substituted with 1 or 2 substituents independently selected from R¹⁰;
R³ is selected from H, methyl, ethyl, phenyl, 1,2,4-triazolyl, pyrazyl, and pyridyl; wherein said methyl, phenyl, 1,2,4-triazolyl, pyrazyl, and pyridyl are each optionally substituted with 1, 2 or 3 substituents independently selected from R³⁰;
R⁵ is selected from H and chloro;
R⁶ is selected from pyrrolidinyl, 2-azabicyclo[3.1.0]hexanyl, 2-azabicyclo[2.2.1]heptanyl, and 5-oxo-1,2,3,5-tetrahydroindolizin-3-yl; wherein said pyrrolidinyl, 2-azabicyclo[3.1.0]hexanyl, 2-azabicyclo[2.2.1]heptanyl, and 5-oxo-1,2,3,5-tetrahydroindolizin-3-yl are optionally substituted with 1 or 2 substituents independently selected from R⁶⁰;
R⁷ is fluoro;
Cy² is

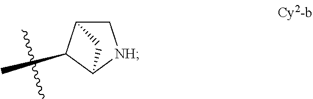

each R¹⁰ is independently selected from methyl, fluoro, and chloro;
each R³⁰ is independently selected from methyl, fluoro, OH, D, and C(O)NR^{c30}R^{d30}; wherein said methyl is optionally substituted with 1 substituent that is R³¹;
each R³¹ is OR^{a31};
each R⁶⁰ is independently selected from methyl, fluoro, C₁₋₂ haloalkoxy, 3-oxomorpholinyl, 2-oxopyrazin-1(2H)-yl), C(O)R^{b60}, C(O)NR⁶⁰R^{d60}, NR^{c60}C(O)R^{b60}, C(O)OR^{a60}, NR^{c60}C(O)OR^{a60}, and NR^{c60}S(O)₂R^{b60}; wherein said 3-oxomorpholinyl, and 2-oxopyrazin-1(2H)-yl) are each optionally substituted with 1 or 2 substituents independently selected from R⁶¹;
each R⁶¹ is independently selected from methyl and fluoro;
each R^{c30} and R^{d30} is independently selected from H and methyl;
each R^{a31} is independently selected from H and methyl; and
each R^{a60}, R^{b60}, R^{c60} and R^{d60} is independently selected from H, C₁₋₂ alkyl, C₁ haloalkyl, cyclopropyl, THFyl, and thiazolyl; wherein said C₁₋₂ alkyl, cyclopropyl, THFyl, and thiazolyl are each optionally substituted with 1 or 2 substituents independently selected from R⁶¹;
or any R^{c60} and R^{d60} attached to the same N atom, together with the N atom to which they are attached, form an azetidinyl group optionally substituted with 1 or 2 substituents independently selected from R⁶¹.

In an embodiment, the compound of Formula I is a compound of Formula II:

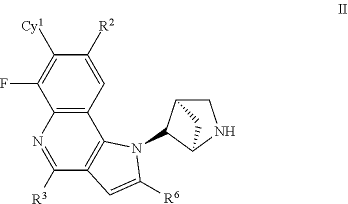

or a pharmaceutically acceptable salt thereof.

In an embodiment of Formula II,
R² is —CH₂CH₂CN;
Cy¹ is phenyl; wherein the phenyl is optionally substituted with 1 or 2 substituents independently selected from R¹⁰;
R³ is selected from H, methyl, and ethyl;
R⁶ is selected from pyrrolidinyl, 2-azabicyclo[3.1.0]hexanyl, 2-azabicyclo[2.2.1]heptanyl, and 5-oxo-1,2,3,5-tetrahydroindolizin-3-yl; wherein said pyrrolidinyl, 2-azabicyclo[3.1.0]hexanyl, 2-azabicyclo[2.2.1]heptanyl, and 5-oxo-1,2,3,5-tetrahydroindolizin-3-yl are optionally substituted with 1 or 2 substituents independently selected from R⁶⁰;
each R¹⁰ is independently selected from methyl, fluoro, and chloro;
each R⁶⁰ is independently selected from methyl, fluoro, C₁₋₂ haloalkoxy, 3-oxomorpholinyl, 2-oxopyrazin-1(2H)-yl), C(O)R^{b60}, C(O)NR⁶⁰R^{d60}, NR^{c60}C(O)R^{b60}, C(O)OR^{a60}, NR^{c60}C(O)OR^{a60}, and NR^{c60}S(O)₂R^{b60}; wherein said 3-oxomorpholinyl, and 2-oxopyrazin-1(2H)-yl) are each optionally substituted with 1 or 2 substituents independently selected from R⁶¹;
each R⁶¹ is independently selected from methyl and fluoro; and
each R^{a60}, R^{b60}, R^{c60} and R^{d60} is independently selected from H, C₁₋₂ alkyl, C₁ haloalkyl, cyclopropyl, THFyl, and thiazolyl.

In another embodiment, the compound of Formula I is a compound of Formula III:

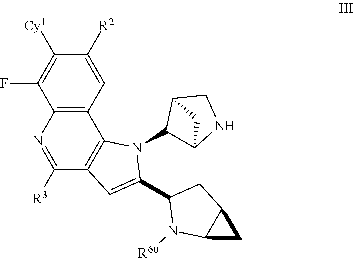

or a pharmaceutically acceptable salt thereof.

In an embodiment of Formula III,
R² is —CH₂CH₂CN;
Cy¹ is phenyl; wherein the phenyl is optionally substituted with 1 or 2 substituents independently selected from R¹⁰;
R³ is selected from H, methyl, and ethyl;
each R¹⁰ is independently selected from methyl, fluoro, and chloro;
each R⁶⁰ is independently selected from methyl, fluoro, C₁₋₂ haloalkoxy, 3-oxomorpholinyl, 2-oxopyrazin-1(2H)-yl), C(O)R^{b60}, C(O)NR⁶⁰R^{d60}, NR^{c60}C(O)R^{b60}, C(O)OR^{a60}, NR^{c60}C(O)OR^{a60}, and NR^{c60}S(O)₂R^{b60}; and
each R^{a6}, R^{b60}, R^{c60} and R^{d60} is independently selected from H, C₁₋₂ alkyl, C₁ haloalkyl, cyclopropyl, THFyl, and thiazolyl.

In an embodiment of Formula I, or a pharmaceutically acceptable salt thereof, Y is CR⁶. In another embodiment of Formula I, or a pharmaceutically acceptable salt thereof, R¹ is H. In yet another embodiment of Formula I, or a pharmaceutically acceptable salt thereof, Cy¹ is phenyl optionally substituted with 1 or 2 substituents independently selected from halo. In still another embodiment of Formula I, or a pharmaceutically acceptable salt thereof, R³ is methyl. In an embodiment of Formula I, or a pharmaceutically acceptable salt thereof, R⁵ is H. In an embodiment of Formula I, or a pharmaceutically acceptable salt thereof, R⁶ is 2-azabicyclo[3.1.0]hexanyl substituted with R⁶⁰. In another embodiment of Formula I, or a pharmaceutically acceptable salt thereof, R⁷ is fluoro. In yet another embodiment of Formula I, or a pharmaceutically acceptable salt thereof, Cy² is Cy²-b. In still another embodiment, R⁶⁰ is C(O)cyclopropyl.

In an embodiment, the KRAS G12D inhibitor is selected from
3-(1-((1R,4R,5S)-2-azabicyclo[2.1.1]hexan-5-yl)-7-(7-chloro-3-hydroxynaphthalen-1-yl)-6-fluoro-2-methyl-4-(1H-1,2,4-triazol-1-yl)-1H-pyrrolo[3,2-c]quinolin-8-yl)propanenitrile;
3-(1-((1R,4R,5S)-2-azabicyclo[2.1.1]hexan-5-yl)-7-(5,7-difluoro-1H-indol-3-yl)-6-fluoro-2-methyl-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-6-fluoro-7-(6-fluoro-5-methyl-1H-indol-3-yl)-2-methyl-4-((S)-1-((S)-1-methylpyrrolidin-2-yl)ethoxy)-1H-pyrrolo[3,2-c]quinolin-8-yl)propanenitrile;
3-(2-(3-(azetidin-1-yl)-3-oxopropyl)-1-((1R,4R,5S)-2-azabicyclo[2.1.1]hexan-5-yl)-7-(2,3-dichlorophenyl)-6-fluoro-4-((S)-1-((S)-1-methylpyrrolidin-2-yl)ethoxy)-1H-pyrrolo[3,2-c]quinolin-8-yl)propanenitrile;
3-((1-((1R,4R,5S)-2-azabicyclo[2.1.1]hexan-5-yl)-6-fluoro-7-(3-hydroxynaphthalen-1-yl)-8-methyl-4-((S)-1-((S)-1-methylpyrrolidin-2-yl)ethoxy)-1H-pyrrolo[3,2-c]quinolin-2-yl)methyl)oxazolidin-2-one;
8-(1-((1R,4R,5S)-2-azabicyclo[2.1.1]hexan-5-yl)-6-fluoro-2,8-dimethyl-4-((S)-1-((S)-1-methylpyrrolidin-2-yl)ethoxy)-1H-pyrrolo[3,2-c]quinolin-7-yl)-1-naphthonitrile;
1-((2S,4S)-1-acetyl-2-(cyanomethyl)piperidin-4-yl)-7-(8-cyanonaphthalen-1-yl)-6-fluoro-4-((S)-1-((S)-1-methylpyrrolidin-2-yl)ethoxy)-1H-pyrazolo[4,3-c]quinoline-8-carbonitrile;
8-(1-((1R,4R,5S)-2-azabicyclo[2.1.1]hexan-5-yl)-6-fluoro-8-methyl-4-((S)-1-((S)-1-methylpyrrolidin-2-yl)ethoxy)-2-((3-oxomorpholino)methyl)-1H-pyrrolo[3,2-c]quinolin-7-yl)-1-naphthonitrile;
3-(7-(benzo[b]thiophen-3-yl)-1-((1R,4R,5S)-2-azabicyclo[2.1.1]hexan-5-yl)-6-fluoro-4-((S)-1-((S)-1-methylpyrrolidin-2-yl)ethoxy)-2-((2-oxopyrrolidin-1-yl)methyl)-1H-pyrrolo[3,2-c]quinolin-8-yl)propanenitrile;
3-(1-((1R,4R,5S)-2-azabicyclo[2.1.1]hexan-5-yl)-4-(((S)-1-(dimethylamino)propan-2-yl)oxy)-6-fluoro-7-(7-fluoronaphthalen-1-yl)-2-((2-oxopyrrolidin-1-yl)methyl)-1H-pyrrolo[3,2-c]quinolin-8-yl)propanenitrile;
8-(1-((1R,4R,5S)-2-azabicyclo[2.1.1]hexan-5-yl)-8-(2-cyanoethyl)-6-fluoro-2-methyl-4-((S)-1-((S)-1-methylpyrrolidin-2-yl)ethoxy)-1H-pyrrolo[3,2-c]quinolin-7-yl)-1-naphthonitrile;
3-(1-((1R,4R,5S)-2-azabicyclo[2.1.1]hexan-5-yl)-7-(2,3-dichloro-5-hydroxyphenyl)-6-fluoro-2-methyl-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-8-(2-cyanoethyl)-6-fluoro-4-((3-fluoro-1-methylazetidin-3-yl)methoxy)-7-(3-hydroxynaphthalen-1-yl)-1H-pyrrolo[3,2-c]quinolin-2-yl)-N,N-dimethylpropanamide;
3-(1-((1R,4R,5S)-2-azabicyclo[2.1.1]hexan-5-yl)-6-fluoro-7-(3-hydroxynaphthalen-1-yl)-2-methyl-4-(5-methylpyrazin-2-yl)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoronaphthalen-1-yl)-4-methyl-2-((4-methyl-2-oxopiperazin-1-yl)methyl)-1H-pyrrolo[3,2-c]quinolin-8-yl)propanenitrile;
3-(1-((1R,4R,5S)-2-azabicyclo[2.1.1]hexan-5-yl)-7-(2,3-dichloro-5-hydroxyphenyl)-4-ethoxy-6-fluoro-2-((4-isopropyl-2-oxopiperazin-1-yl)methyl)-1H-pyrrolo[3,2-c]quinolin-8-yl)propanenitrile;
3-(1-((1R,4R,5S)-2-azabicyclo[2.1.1]hexan-5-yl)-4-(3-(dimethylamino)-3-methylazetidin-1-yl)-6-fluoro-7-(7-fluoronaphthalen-1-yl)-2-((3-oxomorpholino)methyl)-1H-pyrrolo[3,2-c]quinolin-8-yl)propanenitrile;
3-(1-((1R,4R,5S)-2-azabicyclo[2.1.1]hexan-5-yl)-4-ethoxy-6-fluoro-7-(3-hydroxynaphthalen-1-yl)-2-(1-(3-oxomorpholino)ethyl)-1H-pyrrolo[3,2-c]quinolin-8-yl)propanenitrile;
3-(1-((endo)-2-azabicyclo[2.1.1]hexan-5-yl)-6-fluoro-7-(3-hydroxynaphthalen-1-yl)-4-((S)-1-((S)-1-methylpyrrolidin-2-yl)ethoxy)-2-(pyridin-3-yl)-1H-pyrrolo[3,2-c]quinolin-8-yl)propanenitrile;
3-(2-(3-(azetidin-1-yl)-3-oxopropyl)-1-((1R,4R,5S)-2-azabicyclo[2.1.1]hexan-5-yl)-7-(7,8-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;
3-(2-(3-(azetidin-1-yl)-3-oxopropyl)-1-((1R,4R,5S)-2-azabicyclo[2.1.1]hexan-5-yl)-7-(6,7-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoro-3-hydroxynaphthalen-1-yl)-2-methyl-4-((S)-1-((S)-1-methylpyrrolidin-2-yl)ethoxy)-1H-pyrrolo[3,2-c]quinolin-8-yl)propanenitrile;
1-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrazolo[4,3-c]quinolin-7-yl)isoquinoline-8-carbonitrile;
8-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrrolo[3,2-c]quinolin-7-yl)-1-naphthonitrile;
8-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrazolo[4,3-c]quinolin-7-yl)-1-naphthonitrile;
3-(1-((1R,4R,5S)-2-azabicyclo[2.1.1]hexan-5-yl)-6-fluoro-7-(7-fluoro-3-hydroxynaphthalen-1-yl)-2-methyl-4-(1H-1,2,4-triazol-1-yl)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoronaphthalen-1-yl)-2-methyl-4-((S)-1-((S)-1-methylpyrrolidin-2-yl)ethoxy)-1H-pyrrolo[3,2-c]quinolin-8-yl)propanenitrile;
(2R)-2-(1-((1R,4R,5S)-2-azabicyclo[2.1.1]hexan-5-yl)-8-(2-cyanoethyl)-7-(2,3-dichlorophenyl)-6-fluoro-4-(1H-1,2,4-triazol-1-yl)-1H-pyrrolo[3,2-c]quinolin-2-yl)-N,N-dimethylpyrrolidine-1-carboxamide;
methyl (2R)-2-(1-((1R,4R,5S)-2-azabicyclo[2.1.1]hexan-5-yl)-7-(2-chloro-3-methylphenyl)-8-(2-cyanoethyl)-6-fluoro-4-(1H-1,2,4-triazol-1-yl)-1H-pyrrolo[3,2-c]quinolin-2-yl)pyrrolidine-1-carboxylate;
methyl (1S,3R,5S)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;
3-(1-((1R,4R,5S)-2-azabicyclo[2.1.1]hexan-5-yl)-7-(2,3-dichlorophenyl)-6-fluoro-4-methyl-2-(5-oxo-1,2,3,5-tetrahydroindolizin-3-yl)-1H-pyrrolo[3,2-c]quinolin-8-yl)propanenitrile;
methyl (2R)-2-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)pyrrolidine-1-carboxylate;
methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(methylcarbamoyl)pyridin-3-yl)-1H-pyrrolo[3,2-c]quinolin-2-yl)pyrrolidine-1-carboxylate;
3-(1-((1R,4R,5S)-2-azabicyclo[2.1.1]hexan-5-yl)-7-(2-chloro-3-fluorophenyl)-2-((R)-1-(cyclopropanecarbonyl)pyrrolidin-2-yl)-6-fluoro-4-methyl-1H-pyrrolo[3,2-c]quinolin-8-yl)propanenitrile;
8-(2-((R)-1-acetylpyrrolidin-2-yl)-1-((1R,4R,5S)-2-azabicyclo[2.1.1]hexan-5-yl)-6-fluoro-8-methyl-4-(2-methylpyridin-4-yl)-1H-pyrrolo[3,2-c]quinolin-7-yl)-1,2,3,4-tetrahydronaphthalene-1-carbonitrile;
5-(1-((1R,4R,5S)-2-azabicyclo[2.1.1]hexan-5-yl)-7-(3-chloro-2-methylphenyl)-8-(2-cyanoethyl)-6-fluoro-2-((R)-1-(2-oxopyrazin-1(2H)-yl)ethyl)-1H-pyrrolo[3,2-c]quinolin-4-yl)-N-methylpicolinamide;
3-(1-((1R,4R,5S)-2-azabicyclo[2.1.1]hexan-5-yl)-6-fluoro-7-(7-fluoronaphthalen-1-yl)-4-(6-(2-hydroxypropan-2-yl)pyridin-3-yl)-2-((R)-1-(2-oxopyrazin-1(2H)-yl)ethyl)-1H-pyrrolo[3,2-c]quinolin-8-yl)propanenitrile;
3-(1-((1R,4R,5S)-2-azabicyclo[2.1.1]hexan-5-yl)-7-(3-chloro-2-methylphenyl)-6-fluoro-4-(5-methylpyrazin-2-yl)-2-((R)-1-(2-oxopyrazin-1(2H)-yl)ethyl)-1H-pyrrolo[3,2-c]quinolin-8-yl)propanenitrile;
methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(5-fluoro-6-(methylcarbamoyl)pyridin-3-yl)-1H-pyrrolo[3,2-c]quinolin-2-yl)pyrrolidine-1-carboxylate;
methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
ethyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
3-(1-((1R,4R,5S)-2-azabicyclo[2.1.1]hexan-5-yl)-7-(2,3-dichlorophenyl)-2-((R)-1-(3,3-difluoroazetidine-1-carbonyl)pyrrolidin-2-yl)-6-fluoro-4-(methyl-d3)-1H-pyrrolo[3,2-c]quinolin-8-yl) propanenitrile;
3-(1-((1R,4R,5S)-2-azabicyclo[2.1.1]hexan-5-yl)-7-(2,3-dichlorophenyl)-2-((R)-1-(3,3-difluoroazetidine-1-carbonyl)pyrrolidin-2-yl)-6-fluoro-1H-pyrrolo[3,2-c]quinolin-8-yl)propanenitrile;
3-(1-((1R,4R,5S)-2-azabicyclo[2.1.1]hexan-5-yl)-7-(3-chloro-2-methylphenyl)-6-fluoro-4-(5-methylpyrazin-2-yl)-2-((R)-1-(3-oxomorpholino)ethyl)-1H-pyrrolo[3,2-c]quinolin-8-yl)propanenitrile;
5-(1-((1R,4R,5S)-2-azabicyclo[2.1.1]hexan-5-yl)-8-(2-cyanoethyl)-6-fluoro-7-(7-fluoronaphthalen-1-yl)-2-((R)-1-(3-oxomorpholino)ethyl)-1H-pyrrolo[3,2-c]quinolin-4-yl)-N-methylpicolinamide;
3-(1-((1R,4R,5S)-2-azabicyclo[2.1.1]hexan-5-yl)-6-fluoro-7-(7-fluoronaphthalen-1-yl)-4-(6-(2-hydroxypropan-2-yl)pyridin-3-yl)-2-((R)-1-(3-oxomorpholino)ethyl)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoronaphthalen-1-yl)-4-(5-methylpyrazin-2-yl)-2-((R)-1-(3-oxomorpholino)ethyl)-1H-pyrrolo[3,2-c]quinolin-8-yl)propanenitrile;
methyl (1R,3R,5R)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;
3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-6-fluoro-4-(6-(2-hydroxypropan-2-yl)pyridin-3-yl)-2-((R)-1-(2-oxopyrazin-1(2H)-yl)ethyl)-1H-pyrrolo[3,2-c]quinolin-8-yl)propanenitrile;
methyl (2R,4S)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-4-fluoropyrrolidine-1-carboxylate;
methyl (2R,5R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-5-methylpyrrolidine-1-carboxylate;
methyl (2R)-2-(1-((1R,4R,5S)-2-azabicyclo[2.1.1]hexan-5-yl)-3-chloro-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
4-(1-((1R,4R,5S)-2-azabicyclo[2.1.1]hexan-5-yl)-8-(2-cyanoethyl)-7-(2,3-dichlorophenyl)-6-fluoro-2-((R)-1-(2-oxopyrazin-1(2H)-yl)ethyl)-1H-pyrrolo[3,2-c]quinolin-4-yl)-2-fluoro-N-methylbenzamide;
methyl ((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)carbamate;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-2,2-difluoroacetamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-2,2-difluoroacetamide;
(2S)—N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)TH F-2-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)cyclopropanesulfonamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)thiazole-4-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-N-methylcyclopropanecarboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-methylcyclopropane-1-carboxamide;
3-(1-((1R,4R,5S)-2-azabicyclo[2.1.1]hexan-5-yl)-7-(2,3-dichlorophenyl)-6-fluoro-4-(1-hydroxyethyl)-2-((1R,3R,5R)-2-(1-methylcyclopropane-1-carbonyl)-2-azabicyclo[3.1.0]hexan-3-yl)-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-(1-hydroxyethyl)-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-methyl-1H-pyrrolo[3,2-c]quinolin-8-yl)propanenitrile;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclopropane-1-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclobutane-1-carboxamide;
3-(1-((1R,4R,5S)-2-azabicyclo[2.1.1]hexan-5-yl)-7-(3-chloro-2-methylphenyl)-2-(1-(2,6-dimethyl-3-oxo-2,3-dihydropyridazin-4-yl)ethyl)-6-fluoro-4-methyl-1H-pyrrolo[3,2-c]quinolin-8-yl)propanenitrile;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)pyrimidine-4-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)pyridazine-3-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-3,3-difluoroazetidine-1-carboxamide;
3-(1-((1R,4R,5S)-2-azabicyclo[2.1.1]hexan-5-yl)-7-(2,3-dichlorophenyl)-6-fluoro-4-methyl-2-((R)-1-((1-methyl-1H-pyrazol-4-yl)amino)ethyl)-1H-pyrrolo[3,2-c]quinolin-8-yl)propanenitrile;
5-(1-((1R,4R,5S)-2-azabicyclo[2.1.1]hexan-5-yl)-8-(2-cyanoethyl)-7-(2,3-dichlorophenyl)-6-fluoro-2-((R)-1-(1-fluorocyclopropane-1-carbonyl)pyrrolidin-2-yl)-1H-pyrrolo[3,2-c]quinolin-4-yl)-N,N-dimethylpicolinamide; and
methyl (2R)-2-(1-((1R,4R,5S)-2-azabicyclo[2.1.1]hexan-5-yl)-8-(2-cyanoethyl)-7-(2,3-dichlorophenyl)-4-(4-((dimethylamino)methyl)-2,3-difluorophenyl)-6-fluoro-1H-pyrrolo[3,2-c]quinolin-2-yl)pyrrolidine-1-carboxylate;
   and pharmaceutically acceptable salts thereof.

In another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

In yet another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In still another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”):

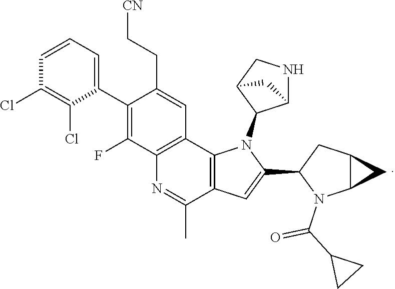

In an embodiment, the KRAS G12D inhibitor is 3-((Sa)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1b”):

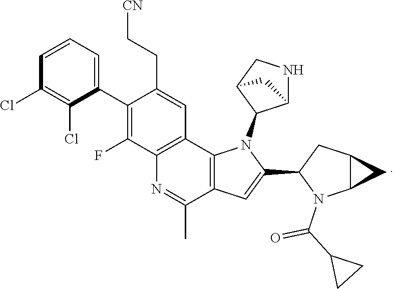

In another aspect, the KRAS G12D inhibitor is a compound of Formula IV:

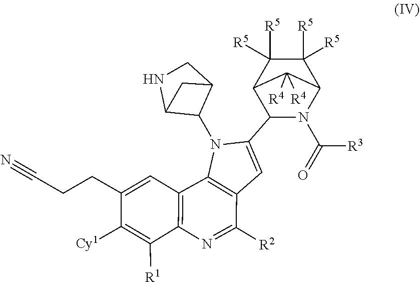

or a pharmaceutically acceptable salt thereof, wherein:
Cy¹ is phenyl optionally substituted with 1, 2, 3, or 4 substituents each selected from D, C₁₋₃ alkyl, C₁₋₃ haloalkyl, C₂₋₃ alkenyl, C₂₋₃ alkynyl, halo, OH, C₁₋₃ alkoxy, and C₁₋₃ haloalkoxy;
R¹ is halogen;
R² is selected from H, D, C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, CN, OR^{a2}, C(O)R^{b2}, C(O)NR^{c2}R^{d2}, NR^{c2}R^{e2}, and NR^{c2}C(O)R^{b2}; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R² are each optionally substituted with 1, 2, or 3 substituents independently selected from R^{2A} wherein the ring-forming atoms of the 4-6 membered heterocycloalkyl, 5-6 membered heteroaryl, C₃₋₆ cycloalkyl-C₁₋₃ alkylene, and 4-6 membered heterocycloalkyl-C₁₋₃ alkylene forming R² consist of at least one carbon atom and 1, 2, 3, or 4 heteroatoms selected from O, N, and S; wherein a ring-forming carbon atom of the 4-6 membered heterocycloalkyl, 5-6 membered heteroaryl, C₃₋₆ cycloalkyl-C₁₋₃ alkylene, and 4-6 membered heterocycloalkyl-C₁₋₃ alkylene forming R² is optionally substituted by oxo to form a carbonyl group; and wherein the C₁₋₃ alkyl, C₂₋₃ alkenyl, and C₂₋₃ alkynyl forming R² are each optionally substituted with 1, 2, or 3 substituents independently selected from R^{2B};
each R^{a2} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a2} are each optionally substituted with 1, 2, or 3 substituents independently selected from R^{2A}; wherein the ring-forming atoms of the 4-6 membered heterocycloalkyl and 5-6 membered heteroaryl forming R^{a2} consist of at least one carbon atom and 1, 2, 3, or 4 heteroatoms selected from O, N, and S; wherein a ring-forming carbon atom of the 4-6 membered heterocycloalkyl and 5-6 membered heteroaryl forming R^{a2} is optionally substituted by oxo to form a carbonyl group; and wherein the C₁₋₆ alkyl, C₂₋₆ alkenyl, and C₂₋₆ alkynyl forming R^{a2} are each optionally substituted with 1, 2, or 3 substituents independently selected from R^{2B} each R^{b2}, R^{c2}, and R^{d2} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b2}, R^{c2}, and R^{d2} are each optionally substituted with 1, 2, or 3 substituents independently selected from R^{2A}; the ring-forming atoms of the 4-6 membered heterocycloalkyl and 5-6 membered heteroaryl forming R^{b2}, R^{c2}, and R^{d2} consist of at least one carbon atom and 1, 2, 3, or 4 heteroatoms selected from O, N, and S; wherein a ring-forming carbon atom of the 4-6 membered heterocycloalkyl and 5-6 membered heteroaryl forming R^{b2}, R^{c2}, and R^{d2} is optionally substituted by oxo to form a carbonyl group; and wherein the C₁₋₃ alkyl, C₂₋₃ alkenyl, and C₂₋₃ alkynyl forming R^{b2}, R^{c2} and R^{d2} are each optionally substituted with 1, 2, or 3 substituents independently selected from R^{2B}; or
any R^{c2} and R^{d2} attached to the same N atom, together with the N atom to which they are attached, form a 4-, 5-, or 6-membered heterocycloalkyl group optionally substituted with 1, 2, or 3 substituents independently selected from R^{2B}
each R^{e2} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e2} are each optionally substituted with 1, 2, or 3 substituents independently selected from R^{2A}; wherein the ring-forming atoms of the 4-6 membered heterocycloalkyl and 5-6 membered heteroaryl forming R^{e2} consist of at least one carbon atom and 1, 2, 3, or 4 heteroatoms selected from O, N, and S; wherein a ring-forming carbon atom of the 4-6 membered heterocycloalkyl and 5-6 membered heteroaryl forming R^{e2} is optionally substituted by oxo to form a carbonyl group; and wherein the C₁₋₃ alkyl, C₂₋₃ alkenyl, and C₂₋₃ alkynyl, forming R^{e2} are each optionally substituted with 1, 2, or 3 substituents independently selected from R^{2B}; or
R^{c2} and R^{e2} attached to the same N atom, together with the N atom to which they are attached, form a 4-, 5-, or 6-membered heterocycloalkyl group optionally substituted with 1, 2, or 3 substituents independently selected from R^{2B};
each R^{2A} is independently selected from C₁₋₃ alkyl, C₂₋₃ alkenyl, C₂₋₃ alkynyl, C₁₋₃ haloalkyl, and R^{2B}, wherein the C₁₋₃ alkyl, C₂₋₃ alkenyl, and C₂₋₃ alkynyl, forming R^{2A} are each optionally substituted with 1, 2 or 3 substituents independently selected from R^{2B};
each R^{2B} is independently selected from C₃₋₆ cycloalkyl, 4-10 membered heterocycloalkyl, phenyl, 5-6 membered heteroaryl, halo, D, CN, OR^{a2}B, C(O)R^{b2B}; C(O)NR^{c2B}R^{d2B}, C(O)OR^{a2B}, NR^{c2B}R^{d2B}, and S(O)₂R^{b2B}; wherein the C₁₋₃ alkyl, C₃₋₆cycloalkyl, 4-6 membered heterocycloalkyl, phenyl, and 5-6 membered heteroaryl are each optionally substituted with 1, 2, or 3 substituents independently selected from R^{2C};
each R^{2C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a2C}, C(O)R^{b2C}, C(O)NR^{c2C}R^{d2C}, C(O)OR^{a2C}, NR^{c2C}R^{d2C}, and S(O)₂R^{b2C};
each R^{a2B}, R^{b2B}, R^{c2B} and R^{d2B} is independently selected from H, C₁₋₃ alkyl, and C₁₋₃ haloalkyl;
each R^{a2C}, R^{b2C}, R^{c2C} and R^{d2C} is independently selected from H, C₁₋₃ alkyl, and C₁₋₃ haloalkyl;
R³ is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, OR^{3A}, and NR^{3B}R^{3C}; wherein the C₃₋₁₀ cycloalkyl, 4-10 membered heterocycloalkyl, C₆₋₁₀ aryl, and 5-10 membered heteroaryl C₁₋₃ alkyl forming R³ are each optionally substituted with 1, 2, or 3 substituents independently selected from R^{3D}; wherein the ring-forming atoms of the 4-10 membered heterocycloalkyl and 5-10 membered heteroaryl forming R³ consist of at least one carbon atom and 1, 2, 3, or 4 heteroatoms selected from O, N, and S; wherein a ring-forming carbon atom of the 4-10 membered heterocycloalkyl or 5-10 membered heteroaryl forming R³ is optionally substituted by oxo to form a carbonyl group; and wherein the C₁₋₃ alkyl, C₂₋₃ alkenyl, and C₂₋₃ alkynyl forming R³ are each optionally substituted with 1, 2, or 3 substituents independently selected from R^{3E};
R^{3A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl; wherein the C₃₋₁₀ cycloalkyl, 4-10 membered heterocycloalkyl, C₆₋₁₀ aryl, and 5-10 membered heteroaryl C₁₋₃ alkyl forming R^{3A} are each optionally substituted with 1, 2, or 3 substituents independently selected from R^{3D}; wherein the ring-forming atoms of the 4-10 membered heterocycloalkyl and 5-10 membered heteroaryl forming R^{3A} consist of at least one carbon atom and 1, 2, 3, or 4 heteroatoms selected from O, N, and S; wherein a ring-forming carbon atom of the 4-10 membered heterocycloalkyl or 5-10 membered heteroaryl forming R^{3A} is optionally substituted by oxo to form a carbonyl group; and wherein the C₁₋₃ alkyl, C₂₋₃ alkenyl, and C₂₋₃ alkynyl forming R^{3A} are each optionally substituted with 1, 2, or 3 substituents independently selected from R^{3E};
R^{3B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl; wherein the C₃₋₁₀ cycloalkyl, 4-10 membered heterocycloalkyl, C₆₋₁₀ aryl, and 5-10 membered heteroaryl forming R^{3B} are each optionally substituted with 1, 2, or 3 substituents independently selected from R^{3D}; wherein the ring-forming atoms of the 4-10 membered heterocycloalkyl and 5-10 membered heteroaryl forming R^{3B} consist of at least one carbon atom and 1, 2, 3, or 4 heteroatoms selected from O, N, and S; wherein a ring-forming carbon atom of the 4-10 membered heterocycloalkyl and 5-10 membered heteroaryl forming R^{3B} is optionally substituted by oxo to form a carbonyl group; and wherein the C₁₋₃ alkyl, C₂₋₃ alkenyl, and C₂₋₃ alkynyl forming R^{3B} are each optionally substituted with 1, 2, or 3 substituents independently selected from R^{3E};
R^{3B} and R^{3C}, together with the N atom to which they are both attached, optionally form a 4-, 5-, 6-, or 7-membered heterocycloalkyl group that is optionally substituted with 1, 2, or 3 substituents independently selected from independently selected from R^{3D};
R^{3C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{3C} are each optionally substituted with 1, 2, or 3 substituents independently selected from R^{3E};
each R^{3D} is independently selected from C₁₋₃ alkyl, C₁₋₃ haloalkyl, C₂₋₃ alkenyl, C₂₋₃ alkynyl, and R^{3E}; wherein each of the C₁₋₃ alkyl, C₂₋₃ alkenyl, and C₂₋₃ alkynyl forming R^{3D} is optionally substituted with 1, 2, or 3 substituents independently selected from R^{3E};
each R^{3E} is independently selected from D, halo, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3} NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3} S(O)R^{b3}, S(O)NR^{c3}R^{d3}, S(O)₂R^{b3}, NR^{c3}S(O)₂R^{b3}, and S(O)₂NR^{c3}R^{d3};
R^{a3}, R^{b3}, R^{c3}, and R^{d3} are each independently selected from H, C₁₋₃ alkyl, C₂₋₃ alkenyl, C₂₋₃ alkynyl, C₆₋₁₀ aryl, C₃₋₇ cycloalkyl, 5-10 membered heteroaryl, 4-10 membered heterocycloalkyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a3}, R^{b3}, R^{c3}, and R^{d3} are each optionally substituted with 1, 2, 3, 4, or 5 substituents independently selected from C₁₋₃ alkyl, C₁₋₃ haloalkyl, C₂₋₃ alkenyl, C₂₋₃ alkynyl, halo, CN, OR^{a3A} SR^{a3A} C(O)R^{b3A}, C(O)NR^{c3A}R^{d3A} C(O)OR^{a3A} OC(O)R^{b3A}, OC(O)NR^{c3A}R^{d3A}, NR^{c3A}R^{d3A}, NR^{c3A}C(O)R^{b3A}, NR^{c3A}C(O)NR^{c3A}R^{d3A}, NR^{c3A}C(O)OR^{a3A}, C(═NR^{e3A})NR^{c3A}R^{d3A}, NR^{c3A}C(═NR^{e3A})NR^{c3A}R^{d3A}, S(O)R^{b3A}, S(O)NR^{c3A}R^{d3A}, S(O)₂R^{b3A}, NR^{c3A}S(O)₂R^{b3A} and S(O)₂NR^{c3A}R^{d3A}; wherein the ring-forming atoms of the 4-10 membered heterocycloalkyl, 5-10 membered heteroaryl, 5-10 membered heteroaryl-C₁₋₃ alkyl, and 4-10 membered heterocycloalkyl-C₁₋₃ alkyl forming R^{a3}, R^{b3}, R^{c3}, and R^{d3} consist of at least one carbon atom and 1, 2, 3, or 4 heteroatoms selected from O, N, and S; wherein a ring-forming carbon atom of the 4-10 membered heterocycloalkyl, 5-10 membered heteroaryl, 5-10 membered heteroaryl-C₁₋₃ alkyl, and 4-10 membered heterocycloalkyl-C₁₋₃ alkyl forming R^{a3}, R^{b3}, R^{c3}, and R^{d3} is optionally substituted by oxo to form a carbonyl group; or
R^{c3} and R^{d3} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{a3A} SR^{a3A}C(O)R^{b3A}, C(O)NR^{c3A}R^{d3A}, C(O)OR^{a3A}, OC(O)R^{b3A}, OC(O)NR^{c3A}R^{d3A}, NR^{c3A}R^{d3A}, NR^{c3A}C(O)R^{b3A}, NR^{c3A}C(O)NR^{c3A}R^{d3A}, NR^{c3A}C(O)OR^{a3A}, C(═NR^{e3A})NR^{c3A}R^{d3A}, NR^{c3A}C(═NR^{e3A})NR^{c3A}R^{d3A}, S(O)R^{b3A}, S(O)NR^{c3A}R^{d3A}, S(O)₂R^{b3A}, NR^{c3A}S(O)₂R^{b3A}, and S(O)₂NR^{c3A}R^{d3A};
R^{a3A}, R^{b3A}, R^{c3A} and R^{d3A} are each independently selected from H, C₁₋₃ alkyl, C₁₋₃ haloalkyl, C₂₋₃ alkenyl, C₂₋₃ alkynyl, aryl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₆ alkyl, C₁₋₆ haloalkyl, C₂₋₆ alkenyl, C₂₋₆ alkynyl C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a3A}, R^{b3A}, R^{c3A} and R^{d3A} are each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy; wherein the ring-forming atoms of the 4-10 membered heterocycloalkyl, 5-10 membered heteroaryl, 5-10 membered heteroaryl-C₁₋₃ alkyl, and 4-10 membered heterocycloalkyl-C₁₋₃ alkyl forming R^{a3A}, R^{b3A}, R^{c3A}, and R^{d3A} consist of at least one carbon atom, and 1, 2, 3, or 4 heteroatoms selected from O, N, and S; and wherein a ring-forming carbon atom of the 4-10 membered heterocycloalkyl, 5-10 membered heteroaryl, 5-10 membered heteroaryl-C₁₋₃ alkyl, and 4-10 membered heterocycloalkyl-C₁₋₃ alkyl forming R^{a3A}, R^{b3A}, R^{c3A}, and R^{d3A} is optionally substituted by oxo to form a carbonyl group; or
R^{c3A} and R^{d3A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy; R^{e3}, and R^{e3A} are each, independently, H, CN or NO₂;
each R⁴ is independently selected from H, D, C₁₋₃ alkyl, C₁₋₃ haloalkyl, C₂₋₃ alkenyl, C₂₋₃ alkynyl, halo, and OR^{a4};
each R^{a4} is independently selected from H, C₁₋₃ alkyl, and C₁₋₃ haloalkyl;
one R⁵ is R^{5A}; and each other R⁵ is independently selected from H, D, halo, C₁₋₃ alkyl, OR^{a5}, C₁₋₃ haloalkyl, C₂₋₃ alkenyl, and C₂₋₃ alkynyl; or, optionally, two other R⁵ attached to the same carbon atom, together with the carbon atom to which they are both attached, form a spiro C₃₋₆ cycloalkyl ring that is optionally substituted with 1, 2, 3, or 4 substituents each selected from D, C₁₋₃ alkyl, and halo; or, optionally, two other R⁵ attached to adjacent carbon atoms, together with the carbon atoms to which they are each attached, form a fused C₃₋₆ cycloalkyl ring that is optionally substituted with 1, 2, 3, or 4 substituents each selected from D, C₁₋₃ alkyl, and halo;
R^{5A} is H, D, C₁₋₃ alkyl, C₁₋₃ haloalkyl, C₂₋₃ alkenyl, C₂₋₃ alkynyl, halo, OR^{a5A}, CN, or Cy²; wherein the C₁₋₃ alkyl forming R^{5A} is optionally substituted with 1, 2, 3 or 4 substituents each selected from R^{5B} and also optionally substituted with Cy², or, optionally, R^{5A} and R⁵ attached to the same carbon atom, together with the carbon atom to which they are both attached, form a spiro C₃₋₆ cycloalkyl ring that is optionally substituted with 1, 2, 3, or 4 substituents each selected from D, C₁₋₃ alkyl, and halo; or, optionally, R^{5A} and R⁵ attached to adjacent carbon atoms, together with the carbon atoms to which they are each attached, form a fused C₃₋₆ cycloalkyl ring that is optionally substituted with 1, 2, 3, or 4 substituents each selected from D, C₁₋₃ alkyl, and halo;
each R^{5B} is independently selected from D and halo;
each R^{a5} is independently selected from H, C₁₋₃ alkyl, and C₁₋₃ haloalkyl;
R^{a5A} is selected from H, C₁₋₃ alkyl, C₁₋₃ haloalkyl, and Cy², wherein the C₁₋₃ alkyl forming R^{a5A} is optionally substituted with 1, 2, 3 or 4 substituents each selected from R^{5B} and also optionally substituted with Cy²;
Cy² is selected from C₃₋₇ cycloalkyl, 4-10 membered heterocycloalkyl, C₆₋₁₀ aryl, and 5-10 membered heteroaryl; wherein the C₃₋₇ cycloalkyl, 4-10 membered heterocycloalkyl, C₆₋₁₀ aryl, 5-10 membered heteroaryl, C₆₋₁₀ aryl, and 5-10 membered heteroaryl forming Cy² is optionally substituted with 1, 2, 3, or 4 substituents independently selected from R^{Cy2}; wherein the ring-forming atoms of the 4-10 membered heterocycloalkyl and 5-10 membered heteroaryl forming Cy² consist of at least one carbon atom and 1, 2, 3, or 4 heteroatoms independently selected from N, O, and S; and wherein a ring-forming carbon atom of the 4-10 membered heterocycloalkyl and 5-10 membered heteroaryl forming Cy² is optionally substituted by oxo to form a carbonyl group;
each R^{Cy2} is independently selected from D, C₁₋₃ alkyl, C₁₋₃ haloalkyl, C₂₋₃ alkenyl, C₂₋₃ alkynyl, C₃₋₆ cycloalkyl, 4-10 membered heterocycloalkyl, C₆₋₁₀ aryl, 5-10 membered heteroaryl, halo, CN, OR^{aCy21}, SR^{aCy21}, C(O)R^{bCy21}, C(O)NR^{cCy21}R^{dCy21}, C(O)OR^{aCy21}, OC(O)R^{bCy21}, OC(O)NR^{cCy21}R^{dCy21}, NR^{cCy21}R^{dCy21}, NR^{eCy21}C(O)R^{bCy21}, NR^{cCy21}C(O)NR^{cCy21}R^{dCy21}, NR^{cCy21}C(O)OR^{aCy21}, C(═NR^{eCy21})NR^{cCy21}R^{dCy21}, NR^{cCy21}C(═NR^{eCy21})NR^{cCy21}R^{dCy21}, S(O)R^{bCy21}, S(O)NR^{cCy21}R^{dCy21}, S(O)₂R^{bCy21}, NR^{cCy21}S(O)₂R^{bCy21}, and S(O)₂NR^{cCy21}R^{dCy21}; wherein the C₃₋₆ cycloalkyl, 4-10 membered heterocycloalkyl, C₆₋₁₀ aryl, and 5-10 membered heteroaryl forming R^{Cy2} are each optionally substituted by 1, 2, 3, or 4 substituents independently selected from R^{Cy2A}; wherein the ring-forming atoms of the 4-10 membered heterocycloalkyl and 5-10 membered heteroaryl forming R^{Cy2} consist of at least one carbon atom and 1, 2, 3, or 4 heteroatoms independently selected from N, O, and S; wherein a ring-forming carbon atom of the 4-10 membered heterocycloalkyl and 5-10 membered heteroaryl forming R^{Cy2} is optionally substituted by oxo to form a carbonyl group; and wherein the C₁₋₃ alkyl, C₂₋₃ alkenyl, and C₂₋₃ alkynyl forming R^{Cy2} are each optionally substituted by 1, 2, or 3 substituents independently selected from R^{Cy2B};
each R^{Cy2A} is independently selected from C₁₋₃ alkyl, C₁₋₃ haloalkyl, C₂₋₃ alkenyl, C₂₋₃ alkynyl, and R^{Cy2B}; wherein the C₁₋₃ alkyl, C₂₋₃ alkenyl, and C₂₋₃ alkynyl forming R^{Cy2A} are each optionally substituted by 1, 2, or 3 substituents independently selected from R^{Cy2B},
each R^{Cy2B} is independently selected from D, halo, CN, OR^{aCy21}, SR^{aCy21}, C(O)R^{bCy21}, C(O)NR^{cCy21}R^{dCy21}, C(O)OR^{aCy21}, OC(O)R^{bCy21}, OC(O)NR^{cCy21}R^{dCy21}, NR^{cCy21}R^{dCy21}, NR^{cCy21}C(O)R^{bCy21}, NR^{cCy21}C(O)NR^{cCy21}R^{dCy21}, NR^{cCy21}C(O)OR^{aCy21}, C(═NR^{eCy21})NR^{cCy21}R^{dCy21}, NR^{cCy21}C(═NR^{eCy21})NR^{cCy21}R^{dCy21}, S(O)R^{bCy21}, S(O)NR^{cCy21}R^{dCy21}, S(O)₂R^{bCy21}, NR^{cCy21}S(O)₂R^{bCy21}, and S(O)₂NR^{cCy21}R^{dCy21},
R^{aCy21}, R^{bCy21}, R^{cCy21}, and R^{dCy21} are each independently selected from H, C₁₋₃ alkyl, C₁₋₃ haloalkyl, C₂₋₃ alkenyl, C₂₋₃ alkynyl, C₆₋₁₀ aryl, C₃₋₇ cycloalkyl, 5-10 membered heteroaryl, 4-10 membered heterocycloalkyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{aCy21}, R^{bCy21}, R^{cCy21}, and R^{dCy21} are each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{aCy22}, SR^{aCy22}, C(O)R^{bCy22}, C(O)NR^{cCy22}R^{dCy2}, C(O)OR^{aCy22}C(O)R^{bCy22}, OC(O)NR^{cCy22}R^{dCy22}, NR^{cCy22}R^{dCy22}, NR^{cCy22}C(O)R^{bCy22}, NR^{cCy22}C(O)NR^{cCy22}R^{dCy22}, NR^{cCy22}C(O)OR^{aCy22}, C(═NR^{eCy22})NR^{cCy22}R^{dCy22}, NR^{eCy22}C(═NR^{eCy22})NR^{cCy22}R^{dCy22}, S(O)R^{bCy22}, S(O)NR^{cCy22}R^{dCy22}, S(O)₂R^{bCy22}, NR^{eCy22}S(O)₂R^{bCy22}, and S(O)₂NR^{Cy22}R^{dCy22}; wherein the ring-forming atoms each of the 5-10 membered heteroaryl, 4-10 membered heterocycloalkyl, 5-10 membered heteroaryl-C₁₋₃ alkyl, and 4-10 membered heterocycloalkyl-C₁₋₃ alkyl forming R^{aCy21}, R^{bCy21}, R^{cCy21}, and R^{dCy21}, consist of at least one carbon atom and 1, 2, 3, or 4 heteroatoms independently selected from N, O, and S; and wherein a ring-forming carbon atom of the 5-10 membered heteroaryl, 4-10 membered heterocycloalkyl, 5-10 membered heteroaryl-C₁₋₃ alkyl, and 4-10 membered heterocycloalkyl-C₁₋₃alkyl forming R^{aCy21}, R^{bCy21}, R^{cCy21}, and R^{dCy21} is optionally substituted by oxo to form a carbonyl group;
or R^{cCy21} and R^{dCy21} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{aCy22}, SR^{aCy22}, C(O)R^{bCy22}, C(O)NR^{cCy22}R^{dCy22}, C(O)OR^{aCy22}, OC(O)RbCy²², OC(O)NR^{cCy22}R^{dCy22}, NR^{cCy22}R^{dCy22}, NR^{cCy22}C(O)R^{bCy22}, NR^{cCy22}C(O)NR^{cCy22}R^{dCy22} NR^{cCy22}C(O)OR^{aCy22}, C(═NR^{eCy22})NR^{cCy22}R^{dCy22}, NR^{cCy22}C(═NR^{eCy22})NR^{cCy22}R^{dCy22} S(O)R^{bCy22}, S(O)NR^{Cy22}R^{dCy22}, S(O)₂R^{bCy22}, NR^{eCy22}S(O)₂R^{bCy22}, and S(O)₂NR^{eCy22}R^{dCy22}, R^{aCy22}, R^{bCy22}, R^{cCy22}, and R^{dCy22} are each independently selected from H, C₁₋₃ alkyl, C₁₋₃ haloalkyl, C₂₋₃ alkenyl, C₂₋₃ alkynyl, aryl, C₆₋₁₀ aryl-C₁₋₃alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₃ alkyl, C₂₋₃ alkenyl, C₂₋₃ alkynyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{aCy22}, R^{bCy22}, R^{cCy22}, and R^{dCy22} are each optionally substituted with 1, 2, or 3 substituents independently selected from OH, CN, amino, NH(C₁₋₃ alkyl), N(C₁₋₃ alkyl)₂, halo, C₁₋₃alkyl, C₁₋₃ alkoxy, C₁₋₃ haloalkyl, and C₁₋₃ haloalkoxy; wherein the ring-forming atoms each of the 5-10 membered heteroaryl, 4-10 membered heterocycloalkyl, 5-10 membered heteroaryl-C₁₋₃ alkyl, and 4-10 membered heterocycloalkyl-C₁₋₃ alkyl forming R^{aCy22}, R^{bCy22}, R^{cCy22}, and R^{dCy22} consist of at least one carbon atom and 1, 2, 3, or 4 heteroatoms independently selected from N, O, and S; and wherein a ring-forming carbon atom of the 5-10 membered heteroaryl, 4-10 membered heterocycloalkyl, 5-10 membered heteroaryl-C₁₋₃ alkyl, and 4-10 membered heterocycloalkyl-C₁₋₃ alkyl forming R^{aCy22}, R^{bCy22}, R^{cCy22}, and R^{dCy22} is optionally substituted by oxo to form a carbonyl group; or
R^{eCy22} and R^{dCy22} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₃alkyl, C₁₋₃ alkoxy, C₁₋₃ haloalkyl, and C₁₋₃ haloalkoxy; and
R^{eCy21} and R^{eCy22} are each, independently, H, CN or NO₂.

In an embodiment of Formula IV,
Cy¹ is phenyl optionally substituted with 1 or 2 substituents each selected from D, C₁₋₃ alkyl, C₁₋₃ haloalkyl, halo, OH, and C₁₋₃ alkoxy;
R¹ is halo;
R² is C₁₋₃ alkyl optionally substituted with OH;
R³ is C₃₋₁₀ cycloalkyl optionally substituted with halo;
each R⁴ is H;
one R⁵ is R^{5A}; and each other R⁵ is independently selected from H, D, halo, C₁₋₃ alkyl, OC₁₋₃ alkyl, C₁₋₃ haloalkyl; or, optionally, two other R⁵ attached to adjacent carbon atoms, together with the carbon atoms to which they are each attached, form a fused C₃₋₆ cycloalkyl ring that is optionally substituted with 1 or 2 substituents each selected from D, C₁₋₃ alkyl, and halo; and
R^{5A} is H, halo, or OR^{a5A},
R^{a5A} is selected from C₁₋₃ alkyl, C₁₋₃ haloalkyl, and Cy², wherein the C₁₋₃ alkyl forming R^{a5A} is optionally substituted with 1, 2, or 3 D, and also optionally substituted with Cy²; and Cy² is selected from C₆₋₁₀ aryl and 5-10 membered heteroaryl.

In another embodiment of Formula IV,
Cy¹ is phenyl optionally substituted with 1 or 2 substituents each selected from D, C₁₋₃ alkyl, C₁₋₃ haloalkyl, halo, OH, and C₁₋₃ alkoxy;
R¹ is halo;
R² is C₁₋₃ alkyl optionally substituted with OH;
R³ is OR^{3A} or C₃₋₁₀ cycloalkyl optionally substituted with halo;
R^{3A} is C₁₋₃ alkyl;
each R⁴ is H;
one R⁵ is R^{5A}; and each other R⁵ is independently selected from H, D, halo, C₁₋₃ alkyl, OC₁₋₃ alkyl, C₁₋₃ haloalkyl; or, optionally, two other R⁵ attached to adjacent carbon atoms, together with the carbon atoms to which they are each attached, form a fused C₃₋₆ cycloalkyl ring that is optionally substituted with 1 or 2 substituents each selected from D, C₁₋₃ alkyl, and halo;
R^{5A} is H, halo, or OR^{a5A};
R^{a5A} is selected from C₁₋₃ alkyl, C₁₋₃ haloalkyl, and Cy², wherein the C₁₋₃ alkyl forming R^{a5A} is optionally substituted with 1, 2, or 3 D, and also optionally substituted with Cy²; and
Cy² is selected from C₆₋₁₀ aryl and 5-10 membered heteroaryl.

In another embodiment of Formula IV,
Cy¹ is phenyl optionally substituted with 1 or 2 substituents each selected from C₁₋₃ alkyl, C₁₋₃ haloalkyl, halo, OH, and C₁₋₃ alkoxy;
R¹ is halo;
R² is C₁₋₃ alkyl optionally substituted with OH;
R³ is OR^{3A} or C₃₋₁₀ cycloalkyl optionally substituted with halo;
R^{3A} is C₁₋₃ alkyl;
each R⁴ is H;
one R⁵ is R^{5A}; and each other R⁵ is independently selected from H, halo, C₁₋₃ alkyl, OC₁₋₃ alkyl, C₁₋₃ haloalkyl;
R^{5A} is H, halo, or OR^{a5A}; and
R^{a5A} is selected from C₁₋₃ alkyl and C₁₋₃ haloalkyl, wherein the C₁₋₃ alkyl forming R^{a5A} is optionally substituted with 1, 2, or 3 D.

In an embodiment, the compound of Formula IV is a compound of Formula IV-A or Formula IV-B:

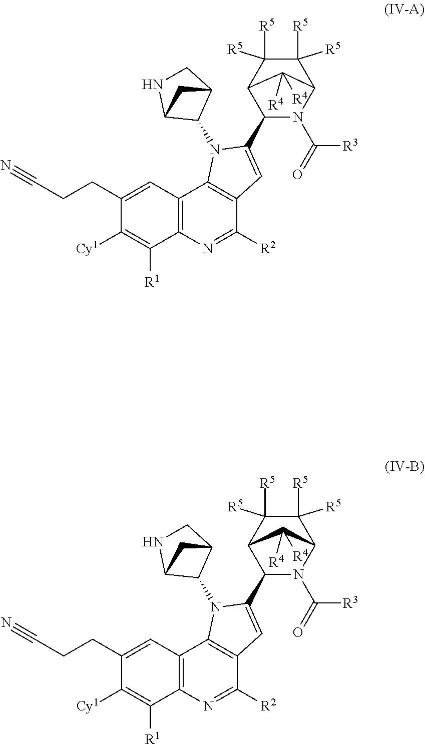

or a pharmaceutically acceptable salt thereof.

In another embodiment, the compound of Formula IV is selected from:
3-(1-(2-azabicyclo[2.1.1]hexan-5-yl)-2-(2-(cyclopropanecarbonyl)-5-methoxy-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-fluoro-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(6-(cyclopropanecarbonyl)-6-azatricyclo[3.2.1.02,4]octan-7-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(methoxy-d3)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(pyridin-3-yloxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(2-(5-(benzyloxy)-2-(cyclopropanecarbonyl)-2-azabicyclo[2.2.1]heptan-3-yl)-1-(2-azabicyclo[2.1.1]hexan-5-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(difluoromethoxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-6-fluoro-2-(5-fluoro-2-(1-fluorocyclopropane-1-carbonyl)-2-azabicyclo[2.2.1]heptan-3-yl)-4-((R)-1-hydroxyethyl)-1H-pyrrolo[3,2-c]quinolin-8-yl)propanenitrile;
3-(1-(2-azabicyclo[2.1.1]hexan-5-yl)-2-(2-(cyclopropanecarbonyl)-5-(difluoromethyl)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
5-(1-(2-azabicyclo[2.1.1]hexan-5-yl)-8-(2-cyanoethyl)-2-(2-(cyclopropanecarbonyl)-2-azabicyclo[2.2.1]heptan-3-yl)-7-(2,3-dichlorophenyl)-6-fluoro-1H-pyrrolo[3,2-c]quinolin-4-yl)-N,N-dimethylpicolinamide;
3-(1-(2-azabicyclo[2.1.1]hexan-5-yl)-2-(2-(cyclopropanecarbonyl)-2-azabicyclo[2.2.1]heptan-3-yl)-7-(2,3-dichlorophenyl)-6-fluoro-4-(6-(2-hydroxypropan-2-yl)pyridin-3-yl)-1H-pyrrolo[3,2-c]quinolin-8-yl)propanenitrile;
4-(1-(2-azabicyclo[2.1.1]hexan-5-yl)-8-(2-cyanoethyl)-2-(2-(cyclopropanecarbonyl)-2-azabicyclo[2.2.1]heptan-3-yl)-7-(2,3-dichlorophenyl)-6-fluoro-1H-pyrrolo[3,2-c]quinolin-4-yl)-2-fluoro-N-methylbenzamide;
3-(1-(2-azabicyclo[2.1.1]hexan-5-yl)-2-(2-(cyclopropanecarbonyl)-5-methyl-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-hydroxy-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(pyridin-2-yloxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-(pyridin-4-yloxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-2-(5-(difluoromethoxy)-2-(1-fluorocyclopropane-1-carbonyl)-2-azabicyclo[2.2.1]heptan-3-yl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-6-fluoro-2-(5-fluoro-2-(1-fluorocyclopropane-1-carbonyl)-2-azabicyclo[2.2.1]heptan-3-yl)-4-(1-hydroxyethyl)-1H-pyrrolo[3,2-c]quinolin-8-yl)propanenitrile;
3-(1-(2-azabicyclo[2.1.1]hexan-5-yl)-2-(5-chloro-2-(1-fluorocyclopropane-1-carbonyl)-2-azabicyclo[2.2.1]heptan-3-yl)-7-(2,3-dichlorophenyl)-6-fluoro-4-(1-hydroxyethyl)-1H-pyrrolo[3,2-c]quinolin-8-yl)propanenitrile;
3-(1-(2-azabicyclo[2.1.1]hexan-5-yl)-2-(2-(cyclopropanecarbonyl)-5-(trifluoromethoxy)-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-2-(5-(difluoromethoxy)-2-(1-fluorocyclopropane-1-carbonyl)-2-azabicyclo[2.2.1]heptan-3-yl)-6-fluoro-4-methyl-1H-pyrrolo[3,2-c]quinolin-8-yl)propanenitrile;
3-(1-(2-azabicyclo[2.1.1]hexan-5-yl)-2-(2-(cyclopropanecarbonyl)-5-cyclopropoxy-2-azabicyclo[2.2.1]heptan-3-yl)-7-(2,3-dichlorophenyl)-6-fluoro-4-methyl-1H-pyrrolo[3,2-c]quinolin-8-yl)propanenitrile;
3-(1-(2-azabicyclo[2.1.1]hexan-5-yl)-7-(2,3-dichlorophenyl)-2-(5-(difluoromethoxy)-2-(1-fluorocyclopropane-1-carbonyl)-2-azabicyclo[2.2.1]heptan-3-yl)-6-fluoro-4-(1-hydroxyethyl)-1H-pyrrolo[3,2-c]quinolin-8-yl)propanenitrile;
3-(1-(2-azabicyclo[2.1.1]hexan-5-yl)-2-(5-cyclopropoxy-2-(1-fluorocyclopropane-1-carbonyl)-2-azabicyclo[2.2.1]heptan-3-yl)-7-(2,3-dichlorophenyl)-6-fluoro-4-(1-hydroxyethyl)-1H-pyrrolo[3,2-c]quinolin-8-yl)propanenitrile;
methyl 3-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(trifluoromethoxy)-2-azabicyclo[2.2.1]heptane-2-carboxylate;
3-(1-(2-azabicyclo[2.1.1]hexan-5-yl)-7-(2,3-dichlorophenyl)-6-fluoro-2-(2-(1-fluorocyclopropane-1-carbonyl)-5-(trifluoromethoxy)-2-azabicyclo[2.2.1]heptan-3-yl)-4-methyl-1H-pyrrolo[3,2-c]quinolin-8-yl)propanenitrile;
methyl 3-(1-(2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethyl)-2-azabicyclo[2.2.1]heptane-2-carboxylate; and
3-(1-(2-azabicyclo[2.1.1]hexan-5-yl)-7-(2,3-dichlorophenyl)-2-(5-(difluoromethyl)-2-(1-fluorocyclopropane-1-carbonyl)-2-azabicyclo[2.2.1]heptan-3-yl)-6-fluoro-4-methyl-1H-pyrrolo[3,2-c]quinolin-8-yl)propanenitrile;
   and pharmaceutically acceptable salts thereof.

In another embodiment, the KRAS G12D inhibitor is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2”), or a pharmaceutically acceptable salt thereof.

In still another embodiment, the KRAS G12D inhibitor is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2a”):

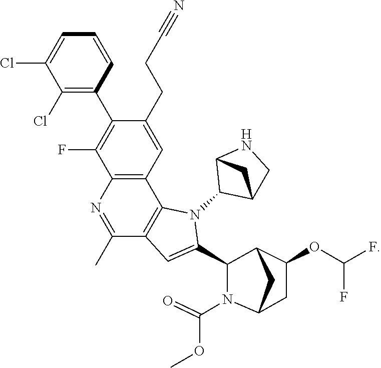

In an embodiment, the KRAS G12D inhibitor is methyl (1R,3R,4R,5S)-3-((Sa)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2b”):

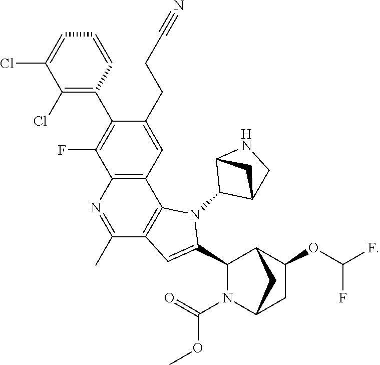

In another embodiment, the KRAS G12D inhibitor is selected from MRTX1133, RMC-9805, HRS-4642, ASP-3082, BI-2852, MRTX-EX185, 3144, QTX3046, VRTX153, JAB-22000, TH-Z827, TH-Z801, TH-Z814, TH-Z816, TH-Z835, TH-Z827, TH-Z837, KID-8, NS-1, and CAS No.: 2765254-39-3.

In another embodiment, the KRAS G12D inhibitor is selected from:

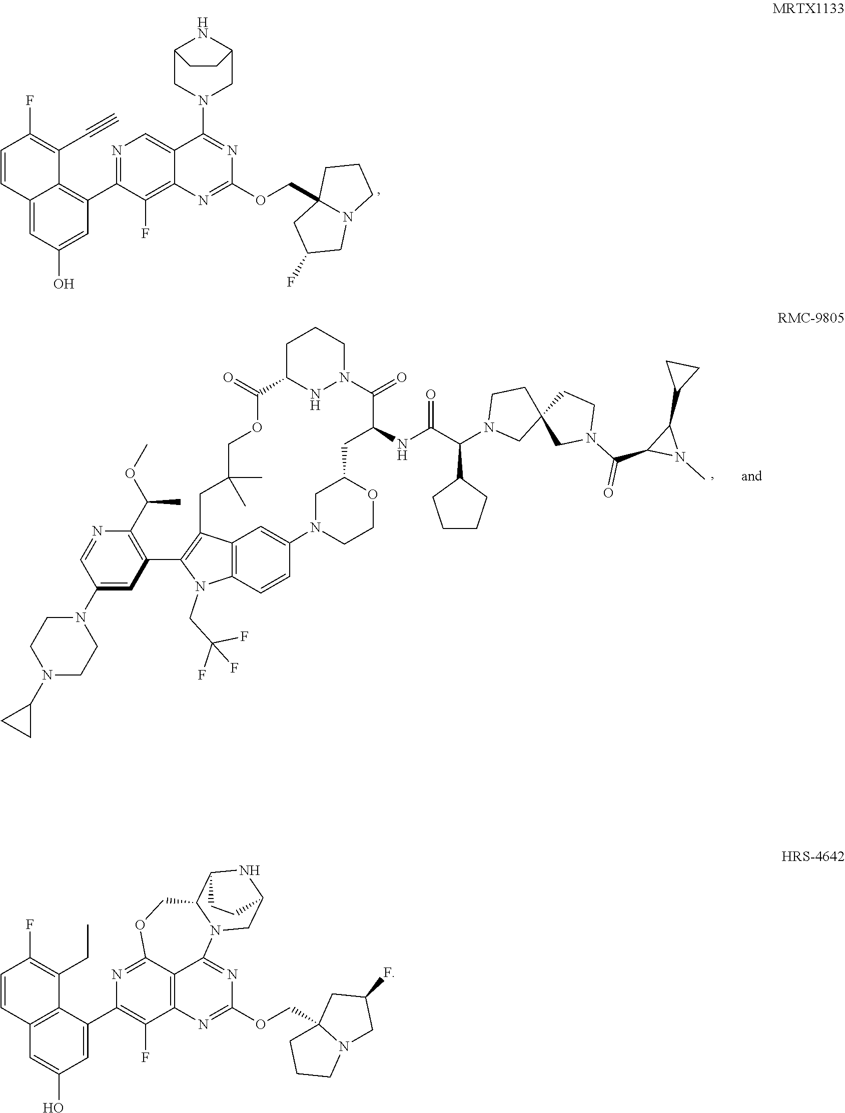

In some embodiments, the inhibitor of KRAS G12D inhibitor is selected from a compound as disclosed in WO2018/145020, WO2022/015375, WO2021/091967, WO2022/060836, US2023/0293464A1, US2023/0219951A1, or US2023/0285498A1, the contents of which are incorporated by reference in their entirety.

In some embodiments, the inhibitor of KRAS G12D inhibitor is selected from a compound as disclosed in WO2024061370A1, the contents of which are incorporated by reference in their entirety.

In some embodiments, the inhibitor of KRAS G12D inhibitor is selected from a compound as disclosed in WO2016161361; WO2020212895; WO2021041671; WO2021081212; WO2021106231; WO2021107160; WO2021126799; WO2021215544; WO2021248079; WO2021248082 WO2021248095; WO2022002102; WO2022015375; WO2022031678; WO2022042630; WO2022066646; WO2022098625; WO2022105855; WO2022105857; WO2022105859; WO2022173033; WO2022177917; WO2022184178; WO2022188729; WO2022192794; WO2022194066; WO2022194191; WO2022194192; WO2022198905; WO2022199170; WO2022199586; WO2022206723; WO2022206724; WO2022212947; WO2022214102; WO2022217042; WO2022221739; WO2022223020; WO2022227987; WO2022228543; WO2022232331; WO2022232332; WO2022234639; WO2022234851; WO2022240971; WO2022261154; WO2022262686; WO2022262838; WO2022266069; WO2022268051; WO2023001123; WO2023001141; WO2023018810; WO2023018812; WO2023020347; WO2023025116; WO2023030495; WO2023051586; WO2023056951; WO2023059594; WO2023059596; WO2023059597; WO2023059598; WO2023059600; WO2023061294; WO2023061463; WO2023072188; WO2023085657; WO2023098425; WO2023098426; WO2023098832; WO2023101928; WO2023103523; WO2023103906; WO2023104018; WO2023113739; WO2023122662; WO2023125627; WO2023125989; WO2023133183; WO2023134465; WO2023143312; WO2023159086; WO2023159087; WO2023179629; WO2023179703; WO2023274324; WO2023274383; WO2023278600; WO2023280026; WO2023280280; WO2023283933; WO2023284537; WO2023284881; U.S. Ser. No. 11/453,683; US20180086752; US20180201610; US20220323614; US20220402971; US20230077225; US20230083431; US20230174518; US20230242544; and US20230279025, the contents of which are incorporated by reference in their entirety.

In yet another embodiment, the KRAS G12D inhibitor is a proteolysis targeting chimera (PROTAC). PROTACs are heterobifunctional compounds comprised of a ligand for a target protein (e.g., KRAS with a G12D mutation) and a ligand for an E3 ligase joined by a linker.

In some embodiments, the inhibitor of KRAS G12D proteolysis targeting chimera (PROTAC) is selected from compounds as disclosed in WO2022148421; WO2022148422; WO2022173032; WO2023077441; WO2023081476; WO2023119677; WO2023120742; WO2023138524; and WO2023171781; the contents of which are incorporated by reference in their entirety.

In one embodiment, the disclosed compounds may exist as tautomers. All tautomers are included within the scope of the compounds presented herein.

Compounds provided herein can exist in the form of atropisomers (i.e., conformational diastereoisomers) that can be stable at ambient temperature and separable, e.g., by chromatography. For example, the compounds of Formula I can exist in the form of atropisomers that are interchangeable by rotation around the bond connecting Cy¹ (or any of the embodiments thereof) to the remainder of the molecule. Reference to the compounds described herein or any of the embodiments is understood to include all such atropisomeric forms of the compounds. Without being limited by any theory, it is understood that, for a given compound, one atropisomer may be more potent as an inhibitor of KRAS (including G12D mutated form of KRAS) than another atropisomer. For example, compounds of formula I as described herein in which Cy¹ is 2,3-dichlorophenyl can exist in the form of atropisomers in which the conformation of the dichlorophenyl relative to the remainder of the molecule is as shown by the partial formulae Formula IV-A or Formula IV-B below. The asymmetry of atropisomers is assigned as either Rₐ or Sₐ, as determined by conventional methods of characterizing points of asymmetry. Without being limited by any theory, it is understood that, for a given compound, the atropisomer represented by Formula IV-A is generally more potent as an inhibitor of KRAS (including G12D mutated forms of KRAS) than the atropisomer represented by Formula IV-B.

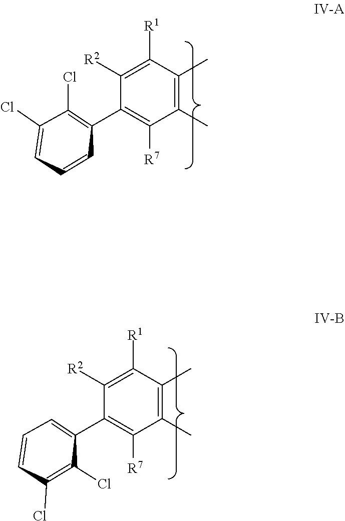

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³Cl, ¹⁸F, ¹²³I, ¹²⁵I, ¹³N, ¹⁵N, ¹⁵O, ¹⁷O, ¹⁸O, ³²P, and ³⁵S. In another embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, the compounds described herein include a ²H (i.e., deuterium) isotope.

In still another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

The specific compounds described herein, and other compounds encompassed by one or more of the formulas described herein having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Carey and Sundberg, Advanced Organic Chemistry 4th Ed., Vols. A and B (Plenum 2000, 2001), Advances in Heterocyclic Chemistry, Vols. 1-114 (Elsevier, 1963-2023); Journal of Heterocyclic Chemistry Vols. 1-60 (Journal of Heterocyclic Chemistry, 1964-2023); E. M. Carreira, et al. (Eds.) Science of Synthesis, Vols. 1-48 (2001-2010) and Knowledge Updates KU2010/1-4; 2011/1-4; 2012/1-4, 2013/1-4; 2014/1-4, 2015/1-2; 2016/1-3, 2017/1-3; 2018/1-4, 2019/1-3; 2020/1-3, 2021/1-3, 2022/1-3, 2023/1 (Thieme, 2001-2023); Houben-Weyl, Methoden der Organischen Chemie, 4^{th} Ed. Vols. 1-67 (Thieme, 1952-1987); Houben-Weyl, Methoden der Organischen Chemie, E-Series. Vols. 1-23 (Thieme, 1982-2003); A. R. Katritzky, et al. (Eds.), Comprehensive Organic Functional Group Transformations, Vols. 1-6 (Pergamon Press, 1995); A. R. Katritzky et al. (Eds.), Comprehensive Organic Functional Group Transformations II, Vols. 1-6 (Elsevier, 2^{nd} Edition, 2005); A. R. Katritzky et al. (Eds.); Comprehensive Heterocyclic Chemistry, Vols. 1-8 (Pergamon Press, 1984); A. R. Katritzky, et al. (Eds.); Comprehensive Heterocyclic Chemistry II, Vols. 1-10 (Pergamon Press, 1996); A. R. Katritzky, et al. (Eds.); Comprehensive Heterocyclic Chemistry III, Vols. 1-14 (Elsevier Science, 2008); D. St. C. Black, et al. (Eds.); Comprehensive Heterocyclic Chemistry IV, Vols. 1-14 (Elsevier Science, 2022); M. B. Smith et al., March's Advanced Organic Chemistry: Reactions, Mechanisms, and Structure, 6^{th} Ed. (Wiley, 2007); M. B. Smith et al., March's Advanced Organic Chemistry: Reactions, Mechanisms, and Structure, 8^{th} Ed. (Wiley, 2020); B. M. Trost et al. (Ed.), Comprehensive Organic Synthesis, Vols. 1-9 (Pergamon Press, 1991); and Patai's Chemistry of Functional Groups, 100 Vols. (Wiley 1964-2022) (all of which are incorporated by reference for such disclosure). General methods for the preparation of compounds as described herein are modified by the use of appropriate reagents and conditions, for the introduction of the various moieties found in the Formulas as provided herein.

Compounds described herein are synthesized using any suitable procedures starting from compounds that are available from commercial sources, or are prepared using procedures described herein.

### Nucleoside Analogs

Nucleoside analogs act as DNA polymerase inhibitors, thereby reducing DNA synthesis. Once phosphorylated, nucleoside analogs act as antimetabolites by being similar enough to natural nucleotides to be incorporated into growing DNA strands. This results in chain termination and stops DNA polymerase. As such, provided herein are combinations comprising a KRAS G12D inhibitor, or a pharmaceutically acceptable salt thereof, a nucleoside analog, or a pharmaceutically acceptable salt thereof, and/or a microtubule inhibitor, or pharmaceutically acceptable salt thereof.

In an embodiment, the nucleoside analog is an adenine analog. In an embodiment, the nucleoside analog is a guanine analog. In an embodiment, the nucleoside analog is a thymine analog. In an embodiment, the nucleoside analog is a cytosine analog. In an embodiment, the nucleoside analog is a uracil analog.

In an embodiment, the nucleoside analog is selected from gemcitabine, cytarabine, azacytidine, cladribine, decitabine, fluorouracil, floxuridine, fludarabine, and nelarabine. In another embodiment, the nucleoside analog is gemcitabine having the following structure:

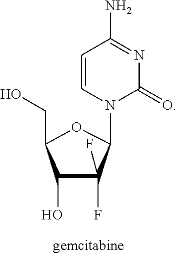

In another embodiment, the nucleoside analog is administered once weekly. In another embodiment, the nucleoside analog is administered as an intraperitoneal injection (IP).

In some embodiments, the nucleoside analog is selected from a compound as disclosed in U.S. Pat. Nos. 2,802,005, 2,885,396, 3,317,384, 4,210,745, 4,405,611, 4,719,295, 4,954,623, 4,526,988, 5,401,838, 5,521,294, WO89/08658, WO91/15498, WO92/01456, or WO93/01202, the contents of which are incorporated by reference in their entirety.

### Microtubule Inhibitors

Microtubules are important cellular targets for anticancer therapy due to their key role in mitosis. Microtubule inhibitors stabilize or destabilize microtubules, thereby suppressing microtubule dynamics required for proper mitotic function, which effectively blocks cell cycle progression resulting in apoptosis. Provided herein are combinations and methods of treatment comprising a KRAS G12D inhibitor, or a pharmaceutically acceptable salt thereof, a nucleoside analog, or a pharmaceutically acceptable salt thereof, and/or a microtubule inhibitor, or pharmaceutically acceptable salt thereof.

In an embodiment, the microtubule inhibitor is a microtubule stabilizer. In another embodiment, the microtubule stabilizer is a taxane. In another embodiment, the microtubule stabilizer is paclitaxel or docetaxel having the following structures:

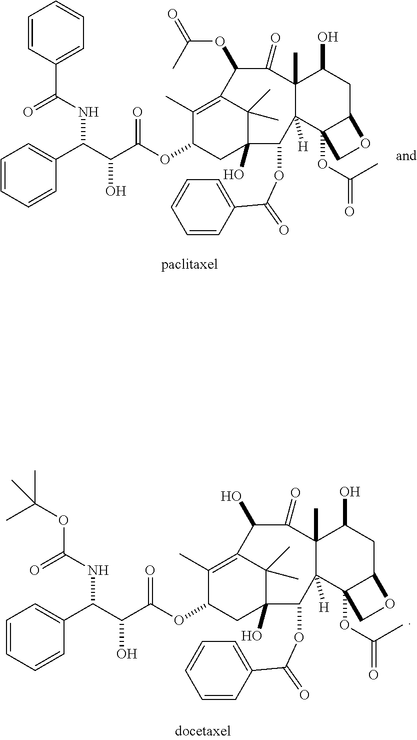

In another embodiment, the microtubule stabilizer is paclitaxel. In another embodiment, the microtubule inhibitor is nanoparticle albumin-bound paclitaxel (nab-paclitaxel).

In another embodiment, the microtubule inhibitor is an epothilone. In another embodiment, the microtubule inhibitor is Epothilone A. In another embodiment, the microtubule inhibitor is Epothilone B. In another embodiment, the microtubule inhibitor is Epothilone C. In another embodiment, the microtubule inhibitor is Epothilone D. In another embodiment, the microtubule inhibitor is Epothilone E. In another embodiment, the microtubule inhibitor is Epothilone F.

In another embodiment, the microtubule inhibitor is administered once weekly. In another embodiment, the microtubule inhibitor is administered as an intraperitoneal injection (IP).

In some embodiments, the microtubule inhibitor is selected from a compound as disclosed in U.S. Pat. Nos. 4,876,399, 4,924,011, 4,960,790, 5,015,744, 5,019,504, 5,824,701, WO91/13066, WO92/09589, WO93/18210, WO93/23389, WO97/19086, WO97/33552, WO98/08849, WO98/25929, WO99/01124, WO99/02514, WO99/43320, and WO2008/057562, the contents of which are incorporated by reference in their entirety.

### Methods of Treatment

In an aspect, provided herein is a method of treating cancer in a subject in need thereof comprising administering to the subject a KRAS G12D inhibitor, or a pharmaceutically acceptable salt thereof, a nucleoside analog, or a pharmaceutically acceptable salt thereof, and/or a microtubule inhibitor, or pharmaceutically acceptable salt thereof.

In an embodiment, the method comprises administering to the subject a KRAS G12D inhibitor, or a pharmaceutically acceptable salt thereof, a nucleoside analog, or a pharmaceutically acceptable salt thereof, and a microtubule inhibitor, or pharmaceutically acceptable salt thereof.

In another aspect, provided herein is a method of treating cancer in a subject in need thereof comprising administering to the subject:
a pharmaceutical composition comprising a KRAS G12D inhibitor, or a pharmaceutically acceptable salt thereof, and at least one pharmaceutically acceptable carrier or excipient;
a pharmaceutical composition comprising a nucleoside analog, or pharmaceutically acceptable salt thereof, and at least one pharmaceutically acceptable carrier or excipient; and/or
a pharmaceutical composition comprising a microtubule inhibitor, or pharmaceutically acceptable salt thereof, and at least one pharmaceutically acceptable carrier or excipient.

In an embodiment, the method comprises administering to the subject:
a pharmaceutical composition comprising a KRAS G12D inhibitor, or a pharmaceutically acceptable salt thereof, and at least one pharmaceutically acceptable carrier or excipient;
a pharmaceutical composition comprising a nucleoside analog, or pharmaceutically acceptable salt thereof, and at least one pharmaceutically acceptable carrier or excipient; and a pharmaceutical composition comprising a microtubule inhibitor, or pharmaceutically acceptable salt thereof, and at least one pharmaceutically acceptable carrier or excipient.

In an embodiment, the KRAS G12D inhibitor has an IC₅₀ of about 100 nM or lower. In another embodiment, the KRAS G12D inhibitor is selective for inhibiting G12D versus wild-type KRAS.

In an embodiment, the KRAS G12D inhibitor is a compound of Formula I, or a pharmaceutically acceptable salt thereof. In another embodiment, the KRAS G12D inhibitor is selected from a compound listed supra. In another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

In yet another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In still another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”).

In an embodiment, the KRAS G12D inhibitor is 3-((Sa)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1b”).

In an embodiment, the KRAS G12D inhibitor is a compound of Formula IV, or a pharmaceutically acceptable salt thereof. In another embodiment, the KRAS G12D inhibitor is selected from a compound of Formula IV listed supra. In another embodiment, the KRAS G12D inhibitor is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2”), or a pharmaceutically acceptable salt thereof.

In another embodiment, the KRAS G12D inhibitor is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2a”), or a pharmaceutically acceptable salt thereof.

In another embodiment, the KRAS G12D inhibitor is methyl (1R,3R,4R,5S)-3-((Sa)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2b”), or a pharmaceutically acceptable salt thereof.

In an embodiment, the nucleoside analog is selected from gemcitabine, cytarabine, azacytidine, cladribine, decitabine, fluorouracil, floxuridine, fludarabine, and nelarabine. In another embodiment, the nucleoside analog is gemcitabine.

In yet another embodiment, the microtubule inhibitor is a microtubule stabilizer. In still another embodiment, the microtubule stabilizer is a taxane. In another embodiment, the microtubule stabilizer is paclitaxel or docetaxel. In another embodiment, the microtubule stabilizer is paclitaxel. In another embodiment, the microtubule inhibitor is nanoparticle albumin-bound paclitaxel (nab-paclitaxel). In another embodiment, the microtubule stabilizer is cabazitaxel.

In another embodiment, the KRAS G12D inhibitor, or a pharmaceutically acceptable salt thereof, is administered in combination with a nucleoside analog, or pharmaceutically acceptable salt thereof, and/or a microtubule stabilizer, or a pharmaceutically acceptable salt thereof.

In yet another aspect, provided herein is a method of treating cancer in a subject in need thereof comprising administering to the subject:
a pharmaceutical composition comprising Compound 1, and at least one pharmaceutically acceptable carrier or excipient;
a pharmaceutical composition comprising gemcitabine, and at least one pharmaceutically acceptable carrier or excipient; and/or
a pharmaceutical composition comprising nab-paclitaxel, and at least one pharmaceutically acceptable carrier or excipient.

In an embodiment, the method comprises administering to the subject:
a pharmaceutical composition comprising Compound 1*, and at least one pharmaceutically acceptable carrier or excipient;
a pharmaceutical composition comprising gemcitabine, and at least one pharmaceutically acceptable carrier or excipient; and
a pharmaceutical composition comprising nab-paclitaxel, and at least one pharmaceutically acceptable carrier or excipient.

In an embodiment, Compound 1* is Compound 1. In another embodiment, Compound 1* is Compound 1b.

In another aspect, provided herein is a method of treating cancer in a subject in need thereof comprising administering to the subject:
a pharmaceutical composition comprising Compound 2, and at least one pharmaceutically acceptable carrier or excipient;
a pharmaceutical composition comprising gemcitabine, and at least one pharmaceutically acceptable carrier or excipient; and
a pharmaceutical composition comprising nab-paclitaxel, and at least one pharmaceutically acceptable carrier or excipient.

In an embodiment, Compound 2 is Compound 2a. In another embodiment, Compound 2 is Compound 2b.

In another embodiment, the KRAS G12D inhibitor is administered to the subject in a pharmaceutical composition comprising the KRAS G12D inhibitor, or a pharmaceutically acceptable salt thereof, and at least one pharmaceutically acceptable carrier or excipient.

In yet another embodiment, the nucleoside analog is administered to the subject in a pharmaceutical composition comprising the nucleoside analog, or a pharmaceutically acceptable salt thereof, and at least one pharmaceutically acceptable carrier or excipient.

In yet another embodiment, the microtubule inhibitor is administered to the subject in a pharmaceutical composition comprising the microtubule inhibitor, or a pharmaceutically acceptable salt thereof, and at least one pharmaceutically acceptable carrier or excipient.

In yet another aspect, provided herein is a method of treating cancer in a subject in need thereof comprising administering to the subject a KRAS G12D inhibitor that is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, a nucleoside analog that is gemcitabine, or a pharmaceutically acceptable salt thereof, and/or a microtubule inhibitor that is nab-paclitaxel, or a pharmaceutically acceptable salt thereof.

In an embodiment, the method comprises administering to the subject 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, gemcitabine, or a pharmaceutically acceptable salt thereof, and nab-paclitaxel, or a pharmaceutically acceptable salt thereof.

In an embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”).

In yet another embodiment, the KRAS G12D inhibitor is 3-((Sa)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1b”).

In another aspect, provided herein is a method of treating cancer in a subject in need thereof comprising administering to the subject a KRAS G12D inhibitor that is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2”), or a pharmaceutically acceptable salt thereof, a nucleoside analog that is gemcitabine, or a pharmaceutically acceptable salt thereof, and a microtubule inhibitor that is nab-paclitaxel, or a pharmaceutically acceptable salt thereof.

In an embodiment, Compound 2 is Compound 2a. In another embodiment, Compound 2 is Compound 2b.

In another embodiment of the methods, the KRAS G12D inhibitor is administered twice daily (BID). In another embodiment, the KRAS G12D inhibitor is administered once daily (QD). In yet another embodiment, the KRAS G12D inhibitor is administered orally (PO).

In an embodiment, the nucleoside analog is administered once weekly. In another embodiment, the nucleoside analog is administered as an intraperitoneal injection (IP).

In an embodiment, the microtubule inhibitor is administered once weekly. In another embodiment, the microtubule inhibitor is administered as an intraperitoneal injection (IP).

In an embodiment, the nucleoside analog and microtubule inhibitor is a combination of gemcitabine and nab-paclitaxel (wherein the combination is referred to as “GEMNabP”).

In an aspect, provided herein is a method of treating cancer in a subject in need thereof comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, a nucleoside analog, or a pharmaceutically acceptable salt thereof, and a microtubule inhibitor, or pharmaceutically acceptable salt thereof.

In another aspect, provided herein is a method of treating cancer in a subject in need thereof comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride, a nucleoside analog, or a pharmaceutically acceptable salt thereof, and a microtubule inhibitor, or pharmaceutically acceptable salt thereof.

In yet another aspect, provided herein is a method of treating cancer in a subject in need thereof comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate, a nucleoside analog, or a pharmaceutically acceptable salt thereof, and a microtubule inhibitor, or pharmaceutically acceptable salt thereof.

In still another aspect, provided herein is a method of treating cancer in a subject in need thereof comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, gemcitabine, or a pharmaceutically acceptable salt thereof, and a microtubule inhibitor, or pharmaceutically acceptable salt thereof.

In an aspect, provided herein is a method of treating cancer in a subject in need thereof comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride, gemcitabine, or a pharmaceutically acceptable salt thereof, and a microtubule inhibitor, or pharmaceutically acceptable salt thereof.

In another aspect, provided herein is a method of treating cancer in a subject in need thereof comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate, gemcitabine, or a pharmaceutically acceptable salt thereof, and a microtubule inhibitor, or pharmaceutically acceptable salt thereof.

In yet another aspect, provided herein is a method of treating cancer in a subject in need thereof comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, a nucleoside analog, or a pharmaceutically acceptable salt thereof, and nab-paclitaxel.

In still another aspect, provided herein is a method of treating cancer in a subject in need thereof comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride, a nucleoside analog, or a pharmaceutically acceptable salt thereof, and nab-paclitaxel.

In another aspect, provided herein is a method of treating cancer in a subject in need thereof comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate, a nucleoside analog, or a pharmaceutically acceptable salt thereof, and nab-paclitaxel.

In an aspect, provided herein is a method of treating cancer in a subject in need thereof comprising administering to the subject a therapeutically effective amount of 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, and GEMNabP.

In another aspect, provided herein is a method of treating cancer in a subject in need thereof comprising administering to the subject a therapeutically effective amount of 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride and GEMNabP.

In another aspect, provided herein is a method of treating cancer in a subject in need thereof comprising administering to the subject a therapeutically effective amount of 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate and GEMNabP.

In yet another embodiment, the cancer is selected from carcinomas, hematological cancers, sarcomas, and glioblastoma. In still another embodiment, the cancer is a cancer comprising abnormally proliferating cells having a KRAS G12D mutation.

In an embodiment, the method further comprises identifying the presence of abnormally proliferating cells having a KRAS G12D mutation.

In another embodiment, the cancer is a hematological cancer selected from myeloproliferative neoplasms, myelodysplastic syndrome, chronic and juvenile myelomonocytic leukemia, acute myeloid leukemia, acute lymphocytic leukemia, and multiple myeloma.

In another embodiment, the cancer is a carcinoma selected from pancreatic, colorectal, lung, bladder, gastric, esophageal, breast, head and neck, cervical, skin, and thyroid carcinomas. In yet another embodiment, the carcinoma is colorectal carcinoma. In still another embodiment, the carcinoma is lung carcinoma. In an embodiment, the carcinoma is pancreatic carcinoma.

In another embodiment, the cancer is colorectal cancer (CRC). In an embodiment, the cancer is microsatellite stable colorectal cancer (MSS CRC).

In yet another embodiment, the cancer is non-small cell lung cancer (NSCLC).

In still another embodiment, the cancer is pancreatic ductal adenocarcinoma (PDAC).

In another aspect, provided herein is a method of treating colorectal cancer in a subject in need thereof comprising administering to the subject a KRAS G12D inhibitor that is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, a nucleoside analog that is gemcitabine, or a pharmaceutically acceptable salt thereof, and/or a microtubule inhibitor that is nab-paclitaxel, or a pharmaceutically acceptable salt thereof.

In an embodiment, the method comprises administering to the subject 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, gemcitabine, or a pharmaceutically acceptable salt thereof, and nab-paclitaxel, or a pharmaceutically acceptable salt thereof.

In an embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”).

In yet another embodiment, the KRAS G12D inhibitor is 3-((Sa)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1b”).

In an embodiment of the methods, the KRAS G12D inhibitor is 3-((Rₐ)-1-((1R,4R,5S)-2-azabicyclo [2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

In an embodiment of the methods, the KRAS G12D inhibitor is 3-((Rₐ)-1-((1R,4R,5S)-2-azabicyclo [2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride.

In another aspect, provided herein is a method of treating pancreatic cancer in a subject in need thereof comprising administering to the subject a KRAS G12D inhibitor that is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, a nucleoside analog that is gemcitabine, or a pharmaceutically acceptable salt thereof, and/or a microtubule inhibitor that is nab-paclitaxel, or a pharmaceutically acceptable salt thereof. In an embodiment, the pancreatic cancer is pancreatic ductal adenocarcinoma (PDAC).

In an embodiment, the method comprises administering to the subject 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, gemcitabine, or a pharmaceutically acceptable salt thereof, and nab-paclitaxel, or a pharmaceutically acceptable salt thereof.

In an embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”).

In yet another embodiment, the KRAS G12D inhibitor is 3-((Sa)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1b”).

In another aspect, provided herein is a method of treating colorectal cancer in a subject in need thereof comprising administering to the subject a KRAS G12D inhibitor that is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2”), or a pharmaceutically acceptable salt thereof, a nucleoside analog that is gemcitabine, or a pharmaceutically acceptable salt thereof, and a microtubule inhibitor that is nab-paclitaxel, or a pharmaceutically acceptable salt thereof.

In an embodiment, Compound 2 is Compound 2a. In another embodiment, Compound 2 is Compound 2b.

In another aspect, provided herein is a method of treating pancreatic cancer in a subject in need thereof comprising administering to the subject a KRAS G12D inhibitor that is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2”), or a pharmaceutically acceptable salt thereof, a nucleoside analog that is gemcitabine, or a pharmaceutically acceptable salt thereof, and a microtubule inhibitor that is nab-paclitaxel, or a pharmaceutically acceptable salt thereof. In an embodiment, the pancreatic cancer is pancreatic ductal adenocarcinoma (PDAC).

In an embodiment, Compound 2 is Compound 2a. In another embodiment, Compound 2 is Compound 2b.

In an embodiment of the methods, the subject in need thereof is treatment naïve (i.e., the subject has had no prior cancer therapy).

In an embodiment, the cancer is metastatic. In another embodiment, the cancer is a solid tumor.

In an embodiment of the methods, the KRAS inhibitor, the nucleoside analog, and the microtubule inhibitor are administered separately.

In another embodiment of the methods, the cancer is a myeloproliferative neoplasm.

In another embodiment of the methods, the cancer is a myelodysplastic syndrome. Myelodysplastic syndromes (MDS) can include hematopoietic stem cell disorders characterized by one or more of the following: ineffective blood cell production, progressive cytopenias, risk of progression to acute leukemia or cellular marrow with impaired morphology and maturation (dysmyelopoiesis). Myelodysplastic syndromes can also include refractory anemia, refractory anemia with ringed sideroblasts, refractory anemia with excess blasts, refractory anemia with excess blasts in transformation and chronic myelomonocytic leukemia.

In yet another embodiment of the methods, the cancer is selected from the group consisting of chronic myeloid leukemia (CML), polycythemia vera (PV), essential thrombocythemia (ET), myelofibrosis (MF), chronic neutrophilic leukemia, chronic eosinophilic leukemia, chronic myelomonocytic leukemia, juvenile myelomonocytic leukemia, hypereosinophilic syndrome, systemic mastocytosis, atypical chronic myelogenous leukemia, acute lymphoblastic leukemia (ALL), and acute myeloid leukemia (AML). In still another embodiment, the cancer is myelofibrosis (MF).

In an embodiment of the methods, the cancer is selected from the group consisting of primary myelofibrosis, post-polycythemia vera myelofibrosis, or post-essential thrombocythemia myelofibrosis.

In another embodiment of the methods, the subject is human.

In yet another embodiment of the methods, the treatment comprises administering the KRAS inhibitor, the nucleoside analog, and the microtubule inhibitor at substantially the same time.

In still another embodiment of the methods, the treatment comprises administering the KRAS inhibitor, the nucleoside analog, and the microtubule inhibitor at different times.

In an embodiment of the methods, the KRAS inhibitor is administered to the subject, followed by administration of the nucleoside analog and the microtubule inhibitor. In another embodiment, the nucleoside analog and the microtubule inhibitor are administered to the subject, followed by administration of the KRAS inhibitor.

In another embodiment of the methods, the KRAS inhibitor and/or the nucleoside analog and the microtubule inhibitor are administered at dosages that would not be effective when one or both of the KRAS inhibitor and the nucleoside analog and the microtubule inhibitor are administered alone, but which amounts are effective in combination.

In an embodiment of the methods, the method involves the administration of a therapeutically effective amount of a combination or composition comprising compounds provided herein, or pharmaceutically acceptable salts thereof, to a subject (including, but not limited to a human or animal) in need of treatment (including a subject identified as in need).

In another embodiment of the methods, the treatment includes co-administering the amount of the KRAS inhibitor and the amount of the nucleoside analog and the microtubule inhibitor. In an embodiment, the amount of the KRAS inhibitor and the amount of the nucleoside analog and the microtubule inhibitor are in a single formulation or unit dosage form. In still other embodiments, the amount of the KRAS inhibitor and the amount of the nucleoside analog and the microtubule inhibitor are in a separate formulations or unit dosage forms.

In the foregoing methods, the treatment can include administering the amount of KRAS inhibitor and the amount of the nucleoside analog and the microtubule inhibitor at substantially the same time or administering the amount of KRAS inhibitor and the amount of the nucleoside analog and the microtubule inhibitor at different times. In some embodiments of the foregoing methods, the amount of KRAS inhibitor and/or the amount the nucleoside analog and the microtubule inhibitor is administered at dosages that would not be effective when one or both of KRAS inhibitor and the nucleoside analog and the microtubule inhibitor are administered alone, but which amounts are effective in combination.

### Pharmaceutical Combinations

In an aspect, provided herein is a pharmaceutical combination comprising a KRAS G12D inhibitor, or a pharmaceutically acceptable salt thereof, a nucleoside analog, or a pharmaceutically acceptable salt thereof, and/or a microtubule inhibitor, or a pharmaceutically acceptable salt thereof.

In an embodiment, the pharmaceutical combination comprises a KRAS G12D inhibitor, or a pharmaceutically acceptable salt thereof, a nucleoside analog, or a pharmaceutically acceptable salt thereof, and a microtubule inhibitor, or a pharmaceutically acceptable salt thereof.

In an embodiment, the pharmaceutical combination can include separate pharmaceutical dosage forms or pharmaceutical compositions that are also sold independently of each other. In another embodiment, the pharmaceutical combination is for simultaneous or sequential use for being jointly active. In another embodiment, the pharmaceutical combination can include the components separately or together in a single unit dose.

In an embodiment, the KRAS G12D inhibitor is a compound of Formula I, or a pharmaceutically acceptable salt thereof. In another embodiment, the KRAS G12D inhibitor is selected from a compound listed supra. In another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

In yet another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In still another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1”).

In an embodiment, the KRAS G12D inhibitor is 3-((Sa)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1b”).

In an embodiment, the KRAS G12D inhibitor is a compound of Formula IV, or a pharmaceutically acceptable salt thereof. In another embodiment, the KRAS G12D inhibitor is selected from a compound of Formula IV listed supra. In another embodiment, the KRAS G12D inhibitor is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2”), or a pharmaceutically acceptable salt thereof.

In another embodiment, the KRAS G12D inhibitor is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2a”), or a pharmaceutically acceptable salt thereof.

In another embodiment, the KRAS G12D inhibitor is methyl (1R,3R,4R,5S)-3-((Sa)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2b”), or a pharmaceutically acceptable salt thereof.

In an embodiment of the pharmaceutical combinations, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

In an embodiment of the pharmaceutical combinations, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride.

In an embodiment of the pharmaceutical combinations, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate.

In an embodiment, the nucleoside analog is selected from gemcitabine, cytarabine, azacytidine, cladribine, decitabine, fluorouracil, floxuridine, fludarabine, and nelarabine. In another embodiment, the nucleoside analog is gemcitabine.

In yet another embodiment, the microtubule inhibitor is a microtubule stabilizer. In still another embodiment, the microtubule stabilizer is a taxane. In another embodiment, the microtubule stabilizer is paclitaxel or docetaxel. In another embodiment, the microtubule stabilizer is paclitaxel. In another embodiment, the microtubule stabilizer is nab-paclitaxel

In another embodiment of the pharmaceutical combinations, the KRAS G12D inhibitor is administered twice daily (BID). In another embodiment, the KRAS G12D inhibitor is administered once daily (QD). In yet another embodiment, the KRAS G12D inhibitor is administered orally (PO).

In an embodiment, the nucleoside analog is administered once weekly. In another embodiment, the nucleoside analog is administered as an intraperitoneal injection (IP).

In an embodiment, the microtubule inhibitor is administered once weekly. In another embodiment, the microtubule inhibitor is administered as an intraperitoneal injection (IP).

The administration of a pharmaceutical combination provided herein may result in a beneficial effect, e.g., a synergistic therapeutic effect, e.g., with regard to alleviating, delaying progression of or inhibiting the symptoms, and may also result in further surprising beneficial effects, e.g., fewer side-effects, an improved quality of life or a decreased morbidity, compared with a monotherapy applying only one of the pharmaceutically active ingredients used in the combination of the invention.

### Pharmaceutical Compositions

In an aspect, provided herein is a pharmaceutical composition comprising
a) a KRAS G12D inhibitor, or a pharmaceutically acceptable salt thereof;
b) a nucleoside analog, or a pharmaceutically acceptable salt thereof;
c) a microtubule inhibitor, or a pharmaceutically acceptable salt thereof; and
d) at least one pharmaceutically acceptable carrier or excipient.

In an embodiment, the KRAS G12D inhibitor is a compound of Formula I, or a pharmaceutically acceptable salt thereof. In another embodiment, the KRAS G12D inhibitor is selected from a compound listed supra. In another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

In yet another embodiment, the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1*”).

In still another embodiment, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1a”).

In an embodiment, the KRAS G12D inhibitor is 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate (“Compound 1b”).

In an embodiment of the pharmaceutical compositions, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

In an embodiment of the pharmaceutical compositions, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride.

In an embodiment of the pharmaceutical compositions, the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate.

In an embodiment, the KRAS G12D inhibitor is a compound of Formula IV, or a pharmaceutically acceptable salt thereof. In another embodiment, the KRAS G12D inhibitor is selected from a compound of Formula IV listed supra. In another embodiment, the KRAS G12D inhibitor is methyl (1R,3R,4R,5S)-3-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2”), or a pharmaceutically acceptable salt thereof.

In another embodiment, the KRAS G12D inhibitor is methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2a”), or a pharmaceutically acceptable salt thereof.

In another embodiment, the KRAS G12D inhibitor is methyl (1R,3R,4R,5S)-3-((Sa)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (“Compound 2b”), or a pharmaceutically acceptable salt thereof.

In an embodiment, the nucleoside analog is selected from gemcitabine, cytarabine, azacytidine, cladribine, decitabine, fluorouracil, floxuridine, fludarabine, and nelarabine. In another embodiment, the nucleoside analog is gemcitabine.

In yet another embodiment, the microtubule inhibitor is a microtubule stabilizer. In still another embodiment, the microtubule stabilizer is a taxane. In another embodiment, the microtubule stabilizer is paclitaxel or docetaxel. In another embodiment, the microtubule stabilizer is paclitaxel. In another embodiment, the microtubule stabilizer is nab-paclitaxel

### Packaged Formulations

Packaged pharmaceutical formulations or pharmaceutical products are included herein. Such packaged formulations include one or more pharmaceutical formulations comprising a combination of a KRAS inhibitor, nucleoside analog, and a microtubule inhibitor. The combination of compounds in formulated form is contained in a container. The package typically contains instructions for using the formulation to treat an animal (typically a human patient) suffering from cancer.

In certain embodiments the packaged pharmaceutical formulation or pharmaceutical product contains the combination of compounds described herein in a container with instructions for administering the dosage forms on a fixed schedule. In some of these embodiments, the combination of compounds is provided in separate unit dosage forms.

In a particular embodiment, the compounds of the combination can be dosed on the same schedule, whether by administering a single formulation or unit dosage form containing all of the compounds of the combination, or by administering separate formulations or unit dosage forms of the compounds of the combination. However, some of the compounds used in the combination may be administered more frequently than once per day, or with different frequencies that other compounds in the combination. Therefore, in one embodiment the packaged pharmaceutical formation contains a formulation or unit dosage form containing all of the compounds in the combination of compounds, and an additional formulation or unit dosage form that includes one of the compounds in the combination of agents, with no additional active compound, in a container, with instructions for administering the dosage forms on a fixed schedule.

The package formulations provided herein include comprise prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the combination of compounds of the invention can be administered alone, as mixtures, or with additional active agents.

### Administration/Dosage/Formulations

In another aspect, provided herein is a pharmaceutical composition or pharmaceutical combination comprising the compounds disclosed herein, together with a pharmaceutically acceptable carrier.

Administration of the combination includes administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agent(s) as compared to the other agent(s) in the combination. Therefore, to permit appropriate dosing, packaged pharmaceutical products may contain one or more dosage forms that contain the combination of agents, and one or more dosage forms that contain one of the combination of agents, but not the other agent(s) of the combination.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In an embodiment, Compound 1 free base equivalent is administered at a dose of about 50 mg to about 2000 mg. In an embodiment, Compound 1 free base equivalent is administered at a dose of about 100 mg to about 1800 mg. In an embodiment, Compound 1 free base equivalent is administered at a dose of about 200 mg to about 1600 mg. In an embodiment, Compound 1 free base equivalent is administered at a dose of about 200 mg to about 1200 mg. In an embodiment, Compound 1 free base equivalent is administered at a dose of about 200 mg to about 1000 mg.

In an embodiment, Compound 1 free base equivalent is administered at a dose of about 25 mg, about 50 mg, about 100 mg, about 200 mg, about 250 mg, about 300 mg, about 350 mg, about 400 mg, about 450 mg, about 500 mg, about 550 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg. In an embodiment, Compound 1 free base equivalent is administered at a dose of about 200 mg.

In an embodiment, Compound 1 is administered once, twice, thrice, or four times daily.

In an embodiment, Compound 1* free base equivalent is administered at a dose of about 50 mg to about 2000 mg. In an embodiment, Compound 1* free base equivalent is administered at a dose of about 200 mg to about 1600 mg. In an embodiment, Compound 1* free base equivalent is administered at a dose of about 200 mg to about 1200 mg. In an embodiment, Compound 1* free base equivalent is administered at a dose of about 200 mg to about 1000 mg.

In an embodiment, Compound 1* free base equivalent is administered at a dose of about 100 mg, about 200 mg, about 250 mg, about 300 mg, about 350 mg, about 400 mg, about 450 mg, about 500 mg, about 550 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg. In an embodiment, Compound 1* free base equivalent is administered at a dose of about 200 mg.

In an embodiment, Compound 1* is administered once, twice, thrice, or four times daily. In an embodiment, Compound 1* is administered once daily (QD). In an embodiment, Compound 1* is administered orally (PO).

In an embodiment, gemcitabine is administered at a dose of about 500 mg/m² to about 1500 mg/m². In an embodiment, gemcitabine is administered at a dose of about 700 mg/m² to about 1200 mg/m². In an embodiment, gemcitabine is administered at a dose of about 500 mg/m², about 600 mg/m², about 700 mg/m², about 800 mg/m², about 900 mg/m², about 1000 mg/m², about 1100 mg/m², about 1200 mg/m², about 1300 mg/m², about 1400 mg/m², or about 1500 mg/m². In an embodiment, gemcitabine is administered at a dose of about 1000 mg/m².

In an embodiment, nab-paclitaxel is administered at a dose of about 100 mg/m² to 200 mg/m². In an embodiment, nab-paclitaxel is administered at a dose of about 100 mg/m² to 150 mg/m². In an embodiment, nab-paclitaxel is administered at a dose of about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², or about 200 mg/m². In an embodiment, nab-paclitaxel is administered at a dose of about 120 mg/m². In an embodiment, nab-paclitaxel is administered at a dose of about 125 mg/m².

In an embodiment, a combination of gemcitabine and nab-paclitaxel (wherein the combination is referred to as “GEMNabP”) is administered on days 1, 8, and 15 of a 28-day cycle. In an embodiment, GEMNabP is administered on days 1 and 15 of a 28-day cycle. In an embodiment, GEMNabP is administered intravenously (IV).

In an embodiment, Compound 2, or a pharmaceutically acceptable salt thereof, is a administered at a dose of about 50 mg to about 2000 mg. In an embodiment, Compound 2, or a pharmaceutically acceptable salt thereof, is administered at a dose of about 200 mg to about 1600 mg. In an embodiment, Compound 2, or a pharmaceutically acceptable salt thereof, is administered at a dose of about 200 mg to about 1200 mg.

In an embodiment, Compound 2, or a pharmaceutically acceptable salt thereof, is administered at a dose of about 100 mg, about 200 mg, about 250 mg, about 300 mg, about 350 mg, about 400 mg, about 450 mg, about 500 mg, about 550 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated using one or more pharmaceutically acceptable excipients or carriers. In one embodiment, the pharmaceutical compositions provided herein comprise a therapeutically effective amount of a disclosed compound and a pharmaceutically acceptable carrier.

The optimum ratios, individual and combined dosages, and concentrations of the drug compounds that yield efficacy without toxicity are based on the kinetics of the active ingredients' availability to target sites, and are determined using methods known to those of skill in the art.

Routes of administration of any of the compositions discussed herein include oral, nasal, rectal, intravaginal, parenteral, buccal, sublingual or topical. The compounds may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

The KRAS inhibitors provided herein, their syntheses, and their biological activity against KRAS can be found in WO 2023/064857, which is incorporated by reference in its entirety. The KRAS inhibitors provided herein, their syntheses, and their biological activity against KRAS can be found in PCT/US2024/025160, which is incorporated by reference in its entirety. The nucleoside analogs provided herein, their syntheses, and their biological activity are described in U.S. Pat. No. 4,808,614; Hertel, L., et al., Journ. Org. Chem. 1988, 53(11), 2406-9; and Wani, M., et al., Journ. Amer. Chem. Soc. 1971, 93(9), 2325-7, the contents of which are hereby incorporated by reference in their entirety.

The microtubule stabilizers provided herein, their syntheses, and their biological activity are described in U.S. Pat. Nos. 4,876,399, 4,924,011, 4,960,790, 5,015,744, 5,019,504, 5,824,701, WO91/13066, WO92/09589, WO93/18210, WO93/23389, WO97/19086, WO97/33552, WO98/08849, WO98/25929, WO99/01124, WO99/02514, WO99/43320, and WO2008/057562, the contents of which are hereby incorporated by reference in their entirety.

The nucleoside analogs stabilizers provided herein, their syntheses, and their biological activity are described in U.S. Pat. Nos. 2,802,005, 2,885,396, 3,317,384, 4,210,745, 4,405,611, 4,719,295, 4,954,623, 4,526,988, 5,401,838, 5,521,294, WO89/08658, WO91/15498, WO92/01456, or WO93/01202, the contents of which are incorporated by reference in their entirety.

The following abbreviations may be used herein: AcOH (acetic acid); Ac₂O (acetic anhydride); AE (adverse event); aq. (aqueous); atm. (atmosphere(s)); BID (twice daily); Boc (t-butoxycarbonyl); BOP ((benzotriazol-1-yloxy)tris(dimethylamino)phosphonium hexafluorophosphate); br (broad); C (cycle); Cbz (carboxybenzyl); calc. (calculated); CT (computed tomography); D (day): d (doublet); dd (doublet of doublets); DBU (1,8-diazabicyclo[5.4.0]undec-7-ene); DCM (dichloromethane); DIAD (N, N′-diisopropyl azidodicarboxylate); DIEA (N,N-diisopropylethylamine); DILI (drug-induced liver injury); DIPEA (N, N-diisopropylethylamine); DIBAL (diisobutylaluminium hydride); DLT (dose limiting toxicity); DMF (N,N-dimethylformamide); DMSO (dimethyl sulfoxide); ECG (electrocardiogram); ECOG (Eastern Cooperative Oncology Group_; Et (ethyl); EtOAc (ethyl acetate); FCC (flash column chromatography); FDG (2-(fluorine 18)Fluoro-2-deoxy-D-glucose); g (gram(s)); h (hour(s)); GEMNabP (gemcitabine/nab-paclitaxel); HATU (N, N, N′, N′-tetramethyl-O-(7-azabenzotriazol-1-yl)uronium hexafluorophosphate); HCl (hydrochloric acid); HPLC (high performance liquid chromatography); HRT (hormone replacement therapy); Hz (hertz); ICF (informed consent form); INR (international normalized ratio); irAE (immune-related adverse event); IVD (in vitro diagnostic); IVDR (in vitro diagnostic regulation); J (coupling constant); L (liter(s)); LCMS (liquid chromatography-mass spectrometry); LDA (lithium diisopropylamide); m (multiplet); M (molar); mCPBA (3-chloroperoxybenzoic acid); MS (Mass spectrometry); Me (methyl); MeCN (acetonitrile); MeOH (methanol); mg (milligram(s)); min. (minutes(s)); mL (milliliter(s)); mmol (millimole(s)); MRI (magnetic resonance imaging); MTBE (methyl tert-butyl ether); MTD (maximum tolerated dose); mTPI (modified toxicity probability interval); N (normal); NCS (N-chlorosuccinimide); NEt₃ (triethylamine); NGS (next-generation sequencing); nM (nanomolar); NMP (N-methylpyrrolidinone); NMR (nuclear magnetic resonance spectroscopy); ORR (objective response rate); OTf (trifluoromethanesulfonate); Ph (phenyl); pM (picomolar); PCR (polymerase chain reaction); PET (positron emission tomography); PO (orally); PK (pharmacokinetics); PPT (precipitate); PT (prothrombin time); PTT (partial thromboplastin time); QD (once daily); QTcF (QT interval corrected using Friderica's formula); RDE (recommended dose for expansion: RECIST (Response Evaluation Criteria in Solid Tumors); RP-HPLC (reverse phase high performance liquid chromatography); r.t. (room temperature), s (singlet); SD (stable disease); t (triplet or tertiary); TBS (tert-butyldimethylsilyl); tert (tertiary); TnI (troponin I); TnT (troponin T); tt (triplet of triplets); TFA (trifluoroacetic acid); THF (tetrahydrofuran); ULN (upper limit of normal); WOCBP (woman of child bearing potential); μg (microgram(s)); μL (microliter(s)); pM (micromolar); wt % (weight percent). Brine is saturated aqueous NaCl. In vacuo is under vacuum.

### Example 1: 3-(1-((1R,4R,5S)-2-Azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile

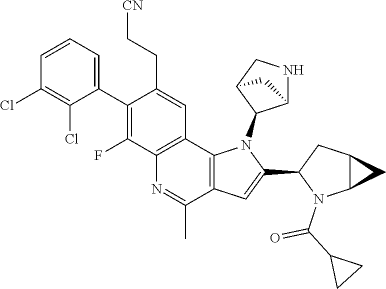 [SMILES: Cc1nc2c(F)c(-c3cccc(Cl)c3Cl)c(CCC#N)cc2c2c1cc([C@H]1C[C@H]3C[C@H]3N1C(=O)C1CC1)n2[C@H]1[C@H]2CN[C@@H]1C2]

### Step 1. Methyl 2-amino-4-bromo-3-fluorobenzoate

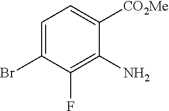 [SMILES: COC(=O)c1ccc(Br)c(F)c1N]

Dimethyl sulfate (823 g, 6.53 mol) was added to a mixture of 2-amino-4-bromo-3-fluorobenzoic acid (1500 g, 6.22 mol) and K₂CO₃ (945 g, 6.84 mol) in N,N-dimethylamide or 1,4-dioxane (6 L) at 5-50° C. After the addition, the mixture was stirred at r.t. for 2 h to complete the reaction. Water (7.5 L) was gradually added to the reaction mixture to precipitate the product. After the water addition, the mixture was stirred at r.t. for 1 h. The solids were isolated by filtration and the wet cake was washed with water (3×1.5 L). The solids were dried under vacuum at about 50° C. overnight to give desired product (1530 g, 99% yield). LCMS calculated for C₃H₇BrFNO₂: 246.96. Found: 248 (M+H⁺). ¹H NMR (400 MHz, DMSO-d₆) δ 7.49 (dd, J=8.8, 1.7 Hz, 1H), 6.87-6.77 (m, 3H), 3.82 (s, 3H). ¹⁹F NMR (376 MHz, DMSO-d₆) δ −127.24

### Step 2. Methyl 3-amino-2′,3′-dichloro-2-fluoro-[1,1′-biphenyl]-4-carboxylate

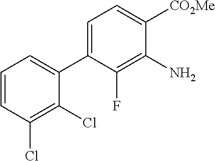 [SMILES: COC(=O)c1ccc(-c2cccc(Cl)c2Cl)c(F)c1N]

Bis(di-tert-butyl(4-dimethylaminophenyl)phosphine)dichloropalladium(II) (Pd-132) (8.12 g, 0.011 mol) was added to a mixture of methyl 2-amino-4-bromo-3-fluorobenzoate (1420 g, 5.72 mol), 2,3-dichlorophenylboronic acid (1226 g, 6.3 mol) and KF (732 g, 12.6 mol) in MeCN (6 L) and water (1.5 L). The mixture was degassed and refilled with N₂ and heated to 70° C. for 1 h to complete the reaction. Water (6 L) was added to the reaction mixture at 50° C. The mixture was cooled to r.t. and stirred for 1 h. The solids were isolated by filtration and the wet cake was washed with 50% MeCN in water (2×2 L) and water (2×2 L). The solids were dried under vacuum at about 50° C. overnight to give desired product (1700 g, 94% yield). LCMS calculated for C₁₄H₉Cl₂FNO₂: 313.01. Found: 314 (M+H⁺). ¹H NMR (400 MHz, DMSO-d₆) δ 7.74 (dd, J=8.0, 1.6 Hz, 1H), 7.64 (dd, J=8.4, 1.4 Hz, 1H), 7.48 (t, J=7.9 Hz, 1H), 7.40 (dd, J=7.9, 1.6 Hz, 1H), 6.70 (s(b), 2H), 6.51 (dd, J=8.3, 6.6 Hz, 1H), 3.86 (s, 3H). ¹⁹F NMR (376 MHz, DMSO-d₆) δ −134.70

### Step 3. Methyl 3-amino-6-bromo-2′,3′-dichloro-2-fluoro-[1,1′-biphenyl]-4-carboxylate

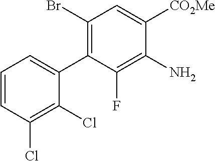 [SMILES: COC(=O)c1cc(Br)c(-c2cccc(Cl)c2Cl)c(F)c1N]

N-Bromosuccinimide (684 g, 3.84 mol) was added to a solution of methyl 3-amino-2′,3′-dichloro-2-fluoro-[1,1′-biphenyl]-4-carboxylate (1150, 3.66 mol) in MeCN (5.75 L) at 50-66° C. After the reaction completion, the MeCN (3 L) was removed by rotavapor. Water (5.75 L) was added to the concentrated mixture and stirred at r.t. for 2-3 h. The solids were isolated by filtration and the wet cake was washed with water to give methyl 3-amino-6-bromo-2′,3′-dichloro-2-fluoro-[1,1′-biphenyl]-4-carboxylate. LCMS calculated for C₁₄H₉BrFCl₂NO₂: 390.92. Found: 391 (M+H). ¹H NMR (400 MHz, DMSO-d₆) δ 7.86 (d, J=1.7 Hz, 1H), 7.79 (dd, J=8.1, 1.5 Hz, 1H), 7.52 (t, J=7.9 Hz, 1H), 7.40 (dd, J=7.7, 1.5 Hz, 1H), 6.83 (s(b), 2H), 3.87 (s, 3H). ¹⁹F NMR (376 MHz, DMSO-d₆) δ −128.19.

### Step 4. 3-Amino-6-bromo-2′,3′-dichloro-2-fluoro-[1,1′-biphenyl]-4-carboxylic acid

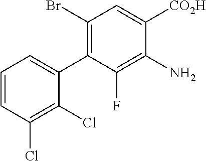 [SMILES: Nc1c(C(=O)O)cc(Br)c(-c2cccc(Cl)c2Cl)c1F]

The wet cake of methyl 3-amino-6-bromo-2′,3′-dichloro-2-fluoro-[1,1′-biphenyl]-4-carboxylate was dissolved in THF (3 L) and MeOH (1.5 L). 1.5 M NaOH (5 L) was added to the solution and the mixture was stirred at about 50° C. for 2 h to complete the saponification reaction. 1.5 M HCl was gradually added to the mixture to adjust the pH to 3-4 and stirred at r.t. for 1 h. The solids were isolated by filtration and the wet cake was washed with water (3×1.2 L). The solids were dried under vacuum at about 50° C. overnight to give desired product (1354 g, 97.5% yield over two steps). LCMS calculated for C₁₃H₇BrCl₂FNO₂: 376.90. Found: 378 (M+H⁺). ¹H NMR (400 MHz, DMSO-d₆) δ 7.85 (d, J=1.7 Hz, 1H), 7.78 (dd, J=8.1, 1.5 Hz, 1H), 7.52 (t, J=7.9 Hz, 1H), 7.39 (dd, J=7.9, 1.5 Hz, 1H), 6.88. ¹⁹F NMR (376 MHz, DMSO-d₆) δ −128.95.

### Step 5. 6-Bromo-7-(2,3-dichlorophenyl)-8-fluoro-2H-benzo[d][1,3]oxazine-2,4(1H)-dione

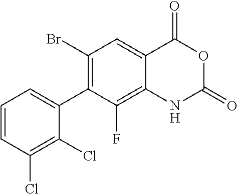 [SMILES: O=c1[nH]c2c(F)c(-c3cccc(Cl)c3Cl)c(Br)cc2c(=O)o1]

Triphosgene (500 g, 1.65 mol) in THF (500 mL) was added to the solution of 3-amino-6-bromo-2′,3′-dichloro-2-fluoro-[1,1′-biphenyl]-4-carboxylic acid (1254 g, 3.31 mol) in THF (4 L) at 60° C. and stirred for 1 h to complete the reaction. The mixture was cooled to 35° C. and n-heptane (10 L) was slowly charged to precipitate the product. The mixture was cooled to r.t. and stirred for 1 h. The solids were isolated by filtration and washed with n-heptane (2×1 L). The wet cake was dried under vacuum at about 50° C. overnight to give desired product (1385 g, quantitative yield). LCMS calculated for C₁₄H₅BrCl₂FNO₃: 402.88. Found: 404 (M+H⁺). ¹H NMR (400 MHz, DMSO-d₆) δ 12.24 (s, 1H), 8.10 (d, J=1.5 Hz, 1H), 7.85 (dd, J=8.1, 1.5 Hz, 1H), 7.58 (t, J=7.9 Hz, 1H), 7.43 (dd, J=7.7, 1.5 Hz, 1H). ¹⁹F NMR (376 MHz, DMSO-d₆) δ −123.98.

### Step 6. Ethyl 6-bromo-7-(2,3-dichlorophenyl)-8-fluoro-4-hydroxy-2-methylquinoline-3-carboxylate

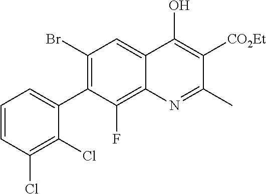 [SMILES: CCOC(=O)c1c(C)nc2c(F)c(-c3cccc(Cl)c3Cl)c(Br)cc2c1O]

A mixture of 6-bromo-7-(2,3-dichlorophenyl)-8-fluoro-2H-benzo[d][1,3]oxazine-2,4(1H)-dione (1078 g, 2.66 mol), ethyl acetoacetate (693 g, 5.32 mol), NaOAc (393 g, 4.79 mol) and NaCl (933 g. 16 mol) in DMSO (5 L) was heated to 50-60° C. for 5 h. The temperature was raised to 100° C. and stirred for 1 h to complete the reaction. The mixture was cooled to about 60° C. and water (10 L) was gradually added to precipitate the product. The mixture was cooled to r.t. and stirred for 1 h. The solids were isolated by filtration and the wet cake was washed with water (2×2 L). The wet solids were dried under vacuum at about 50° C. overnight to give desired product (1145 g, 91% yield). LCMS calculated for C₁₉H₁₃BrCl₂FNO₂: 470.94. Found: 472 (M+H⁺). ¹H NMR (400 MHz, DMSO-d₆) δ 12.05 (s, 1H), 8.18 (d, J=1.5 Hz, 1H), 7.84 (dd, J=8.0, 1.6 Hz, 1H), 7.58 (t, J=7.9 Hz, 1H), 7.50 (dd, J=7.7, 1.6 Hz, 1H), 4.28 (q, J=7.1 Hz, 2H), 2.46 (s, 3H), 1.29 (t, J=7.1 Hz, 3H). ¹⁹F NMR (376 MHz, DMSO-d₆) δ −124.80.

### Step 6a. Ethyl 6-bromo-7-(2,3-dichlorophenyl)-8-fluoro-4-hydroxy-2-methylquinoline-3-carboxylate

The title compound can alternatively be prepared by the following process. A solution of methyl 3-amino-2′,3′-dichloro-2-fluoro-[1,1′-biphenyl]-4-carboxylate (100 g, 0.254 mol), ethyl acetoacetate (33.1 g, 0.51 mol) and p-toluenesulfonic acid (2.2 g, 0.013 mol) in xylene (1 L) was refluxed for 5 h to azeotropically remove water. NaOEt (26 g, 0.381 mol) was added to the mixture and the mixture was refluxed for another 5 h. The mixture was cooled to r.t. and poured into dilute HCl pH=6-7. The organic phase was separated and the aqueous phase was extracted with EtOAc. The combined organic phases were concentrated and the product was purified over silica gel column and eluted with EtOAc and heptane (0-30%) to give desired product (65 g, 54%). LCMS calculated for C₁₉H₁₃BrCl₂FNO₃: 470.91. Found: 472 (M+H⁺). ¹H NMR (400 MHz, DMSO-d₆) δ 12.05 (s, 1H), 8.18 (d, J=1.5 Hz, 1H), 7.84 (dd, J=8.0, 1.6 Hz, 1H), 7.58 (t, J=7.9 Hz, 1H), 7.50 (dd, J=7.7, 1.6 Hz, 1H), 4.28 (q, J=7.1 Hz, 2H), 2.46 (s, 3H), 1.29 (t, J=7.1 Hz, 3H). ¹⁹F NMR (376 MHz, DMSO-d₆) δ −124.80.

### Step 7. Ethyl 6-(2-cyanovinyl)-7-(2,3-dichlorophenyl)-8-fluoro-4-hydroxy-2-methylquinoline-3-carboxylate

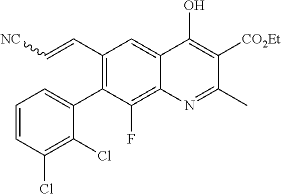 [SMILES: CCOC(=O)c1c(C)nc2c(F)c(-c3cccc(Cl)c3Cl)c(C=CC#N)cc2c1O]

A mixture of ethyl 6-bromo-7-(2,3-dichlorophenyl)-8-fluoro-4-hydroxy-2-methylquinoline-3-carboxylate (246 g, 0.52 mol), acrylonitrile (69 g, 1.3 mol), trimethylamine (156 g, 1.56 mol) and bis(di-tert-butyl)-dimethylaminophenylphosphone dichloride palladium (II) (Pd-132) (14.7 g, 0.02 mol) in N,N-dimethylamide (1.5 L) was heated to 85° C. for about 5 h to complete the reaction. The mixture was cooled to 50° C. and water (1 L) was gradually added. The mixture was cooled to r.t. and 1M HCl was added to adjust the pH to pH 5-6. The solids were isolated by filtration and the wet cake was washed with water (2×500 mL). The wet solids were dissolved in MeOH (1 L) and DCM (9 L). To the solution was added sodium bisulfite (186 g, 1.8 mol) and water (4 L). The mixture was stirred at r.t. for 1 h and the aqueous phase was separated and discarded. The organic phase was washed with water (2×2 L). Activated charcoal (150 g) was added to the organic solution and the mixture was stirred at r.t. for 1 h. The mixture was filtered over a diatomaceous earth bed and the bed was rinsed with DCM (2 L). The organic solution was concentrated to about 1 L and heptane (3.5 L) was gradually added to precipitate the product. The solids were isolated by filtration and washed with heptane (2×2 L). The wet solids were dried under vacuum at about 50° C. overnight to give desired product (210 g, 90% yield). LCMS calculated for C₂₂H₁₅Cl₂FNO₃: 444.04. Found: 445 (M+H⁺). ¹H-NMR (400 MHz, DMSO-d₆) (cis and trans mixture): δ12.05 (s, 1H), 8.64 (s, OH), 8.39 (s, 1H), 7.86 (td, J=7.7, 1.5 Hz, 1H), 7.63-7.53 (m, 1H), 7.47 (td, J=7.5, 1.6 Hz, 1H), 7.04 (d, J=16.5 Hz, 1H), 6.88 (d, J=11.9 Hz, OH), 6.55 (d, J=16.6 Hz, 1H), 5.91 (d, J=12.0 Hz, OH), 4.29 (q, J=7.1 Hz, 2H), 2.47 (d, J=5.0 Hz, 4H), 1.30 (td, J=7.1, 3.2 Hz, 4H).

### Step 8. Ethyl 6-(2-cyanoethyl)-7-(2,3-dichlorophenyl)-8-fluoro-4-hydroxy-2-methylquinoline-3-carboxylate

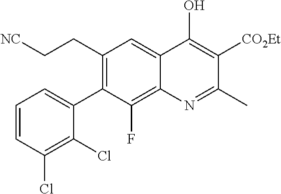 [SMILES: CCOC(=O)c1c(C)nc2c(F)c(-c3cccc(Cl)c3Cl)c(CCC#N)cc2c1O]

A mixture of ethyl 6-(2-cyanovinyl)-7-(2,3-dichlorophenyl)-8-fluoro-4-hydroxyl-2-methylquinoline-3-carboxylate (155 g, 348 mmol), pyridine (450 mL) and 1,4-dioxane (450 mL) was heated to 50-60° C. to give a homogenous solution. To the solution was added NaBH₄ (65.8 g, 1741 mmol) in portions at 50-60° C. The resulting mixture was stirred for 22 h at 50-60° C. to complete the reduction. After cooling to about 15° C., EtOAc (950 mL) was added to the reaction mixture. Concentrated HCl was gradually added to the mixture to adjust the aqueous phase pH to 1-2. The organic phase was separated and the aqueous phase was extracted with EtOAc (500 mL). The combined EtOAc phase was washed with 1N HCl (500 mL), water (2×500 mL), 10% brine (300 mL) and dried over Na₂SO₄ (75 g). The solution was concentrated and the residue was purified by silica gel column (0-20% MeOH in DCM) to give desired product (117.8 g, 76%). LCMS calculated for C₂₂H₁₇Cl₂FN₂O₃: 446.06. Found: 447 (M+H⁺). ¹H NMR (400 MHz, DMSO-d₆) δ 11.87 (s, 1H), 8.00 (s, 1H), 7.84 (dd, J=7.9, 1.7 Hz, 1H), 7.71-7.48 (m, 2H), 4.28 (q, J=7.1 Hz, 2H), 2.79 (ddd, J=11.7, 7.4, 3.7 Hz, 1H), 2.73-2.59 (m, 3H), 2.46 (s, 3H), 1.30 (t, J=7.1 Hz, 3H).

### Step 9. Ethyl 4-chloro-6-(2-cyanoethyl)-7-(2,3-dichlorophenyl)-8-fluoro-2-methylquinoline-3-carboxylate

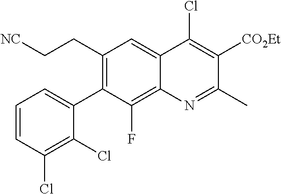 [SMILES: CCOC(=O)c1c(C)nc2c(F)c(-c3cccc(Cl)c3Cl)c(CCC#N)cc2c1Cl]

A mixture of ethyl 6-(2-cyanoethyl)-7-(2,3-dichlorophenyl)-8-fluoro-4-hydroxy-2-methylquinoline-3-carboxylate (60 g, 134 mmol), benzyltriethylammonium chloride (31 g, 135 mmol), N,N-dimethylaniline (49.1 g, 405 mmol) in MeCN (300 mL) was added phosphorus oxychloride (62 g, 405 mmol) at below 20° C. The mixture was heated to 60° C. for 1 h to complete the reaction. The mixture was cooled to r.t. and pooled into ice-water (900 mL) at temperature below 20° C. Product precipitated out during the aqueous quench. The mixture was stirred at r.t. for more than 5 h. The solids were isolated by filtration and the wet cake was washed with 10% MeCN in water (2×150 mL). The wet solids were dried under vacuum at about 50° C. overnight to give desired product (57 g, 90% yield). LCMS calculated for C₂₂H₁₆Cl₃FN₂O₂: 464.03. Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 10. Ethyl 4-chloro-6-(2-cyanovinyl)-7-(2,3-dichlorophenyl)-8-fluoro-2-methylquinoline-3-carboxylate

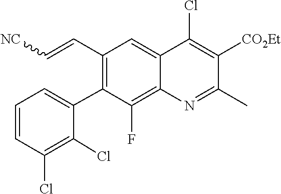 [SMILES: CCOC(=O)c1c(C)nc2c(F)c(-c3cccc(Cl)c3Cl)c(C=CC#N)cc2c1Cl]

A mixture of ethyl 6-(2-cyanovinyl)-7-(2,3-dichlorophenyl)-8-fluoro-4-hydroxy-2-methylquinoline-3-carboxylate (600 g, 1.35 mol), benzyltriethylammonium chloride (307 g, 1.35 mol), N,N-diethylaniline (603 g, 4.04 mol) in MeCN (3 L) was added phosphorus oxychloride (389.7 g, 4.04 mol) at below 20° C. The mixture was heated to 60° C. for 1 h to complete the reaction. The mixture was cooled to r.t. and pooled into ice-water (9 L) at temperature below 20° C. Product precipitated out during the aqueous quench. The mixture was stirred at r.t. for more than 5 h. The solids were isolated by filtration and the wet cake was washed with 10% MeCN in water (2×1.5 L). The wet solids were dried under vacuum at about 50° C. overnight to give desired product (563 g, 90% yield). LCMS calculated for C₂₂H₁₄Cl₃FN₂O₂: 462.01. Found: 463 (M+H⁺). ¹H-NMR (400 MHz, DMSO-d₆) (mixture of cis and trans isomers) δ 8.72 (s, 0.3H), 8.51 (s, 1H), 7.87 (ddd, J=7.3, 5.6, 1.5 Hz, 1.3H), 7.64-7.46 (m, 3H), 7.21 (d, J=16.5 Hz, 1H), 7.05 (d, J=11.9 Hz, 0.3H), 6.73 (d, J=16.5 Hz, 1H), 6.08 (d, J=11.9 Hz, 0.3H), 4.53 (qd, J=7.1, 2.0 Hz, 2H), 2.72 (d, J=7.4 Hz, 4H), 1.41 (t, J=7.1 Hz, 4H).

### Step 11. Ethyl 4-chloro-6-(2-cyanoethyl)-7-(2,3-dichlorophenyl)-8-fluoro-2-methylquinoline-3-carboxylate

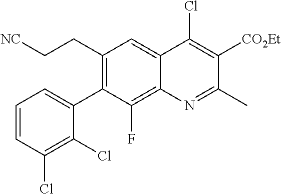 [SMILES: CCOC(=O)c1c(C)nc2c(F)c(-c3cccc(Cl)c3Cl)c(CCC#N)cc2c1Cl]

A mixture of ethyl 4-chloro-6-(2-cyanovinyl)-7-(2,3-dichlorophenyl)-8-fluoro-2-methylquinoline-3-carboxylate (528 g, 1.14 mol) and PMHS (411 g, 6.83 mol) in toluene (1.8 L) were stirred at about 50° C. In another 2-L flask, diacetoxycopper hydrate (4.1 g, 0.02 mol), (9,9-dimethyl-9H-xanthene-4,5-diyl)bis(diphenylphosphane) (13.58 g, 0.023 mol) in toluene (300 ml) and tert-butanol (483 g, 6.52 mol) were stirred for 1-2 h to a solution. The copper acetate solution was slowly added to the solution of ethyl 4-chloro-6-(2-cyanovinyl)-7-(2,3-dichlorophenyl)-8-fluoro-2-methylquinoline-3-carboxylate and PMHS in toluene at 50-60° C. to complete the reduction. The reaction mixture was concentrated under vacuum distillation to about 2 L. To the 2 L residue was added heptane (8 L) at about 50° C. for 1 h. The mixture was cooled to r.t. and stirred overnight. The solids were isolated by filtration and the wet cake was washed with heptane (2×1.2 L). The wet cake and silica gel (260 g) in DCM (2.7 L) were stirred for 1 h. The mixture was filtered over silica gel bed (260 g) and the silica gel bed was rinsed with DCM (4 L) until the eluent was almost colorless. The DCM was removed. DCM (140 mL) and MTBE (260 mL) were added to the residue. The solids were isolated by filtration and the wet cake was washed with MTBE (2×1.2 L). The wet solids were dried under vacuum at about 50° C. overnight to give desired product (476 g, 90% yield). LCMS calculated for C₂₂H₁₆Cl₃FN₂O₂: 464.03. Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 12. Ethyl (Rₐ)-4-chloro-6-(2-cyanoethyl)-7-(2,3-dichlorophenyl)-8-fluoro-2-methylquinoline-3-carboxylate

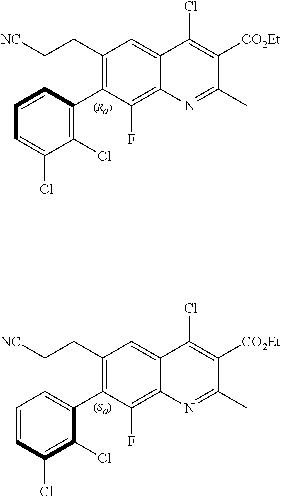

The racemic ethyl 4-chloro-6-(2-cyanoethyl)-7-(2,3-dichlorophenyl)-8-fluoro-2-methylquinoline-3-carboxylate was subject to chiral separation (Chiralpak IB N, MTBE as eluent) to give both ethyl (R)-4-chloro-6-(2-cyanoethyl)-7-(2,3-dichlorophenyl)-8-fluoro-2-methylquinoline-3-carboxylate and ethyl (S)-4-chloro-6-(2-cyanoethyl)-7-(2,3-dichlorophenyl)-8-fluoro-2-methylquinoline-3-carboxylate. LCMS calculated for C₂₂H₁₆Cl₃FN₂O₂: 464.03. Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H)

### Step 13. Ethyl 4-chloro-6-(2-cyanoethyl)-7-(2,3-dichlorophenyl)-8-fluoro-2-methylquinoline-3-carboxylate by racemization

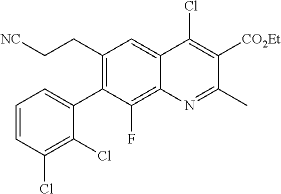 [SMILES: CCOC(=O)c1c(C)nc2c(F)c(-c3cccc(Cl)c3Cl)c(CCC#N)cc2c1Cl]

A mixture of ethyl (S)-4-chloro-6-(2-cyanoethyl)-7-(2,3-dichlorophenyl)-8-fluoro-2-methylquinoline-3-carboxylate (100 g) in sulfolane (200 mL) was heated to 185° C. for 2 h to give racemic ethyl 4-chloro-6-(2-cyanoethyl)-7-(2,3-dichlorophenyl)-8-fluoro-2-methylquinoline-3-carboxylate. The mixture was cooled to 50° C. and MeCN (200 mL) was added. To the solution was added water (700 mL) at 50° C. The mixture was cooled to r.t. and stirred for 4 h. The solids were isolated by filtration and the wet cake was washed with water (2×200 mL). The wet solids were dried under vacuum at about 50° C. overnight to give desired product (97 g, 97% yield). LCMS calculated for C₂₂H₁₆Cl₃FN₂O₂: 464.03. Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H)

### Step 14. tert-Butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

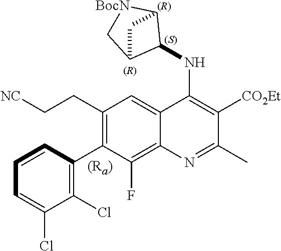

A mixture of ethyl (Rₐ)-4-chloro-6-(2-cyanoethyl)-7-(2,3-dichlorophenyl)-8-fluoro-2-methylquinoline-3-carboxylate (106.3 g, 228 mmol), tert-butyl (1R,4R,5S)-5-amino-2-azabicyclo[2.1.1]hexane-2-carboxylate (58.8 g, 297 mmol), LiCl (19 g, 446 mmol), DIPEA (99.5 g, 670 mmol) in DMSO (400 mL) was heated to 80° C. overnight. The reaction mixture was cooled to r.t. and MTBE (1 L) and water (500 mL) were subsequently added. The organic phase was separated. The organic phase was washed with 0.1 N HCl (500 mL), saturated NaHCO₃ (500 mL) and water (500 mL). The solvent was removed under reduced pressure to give desired product that was used for next step without further purification. Analytical sample was purified by FCC (0-10% MeOH in DCM). LCMS calculated for C₃₂H₃₃Cl₂FN₄O₄: 626.19. Found: 627 (M+H⁺). ¹H-NMR (400 MHz, DMSO-d₆) δ 8.09 (s, 1H), 7.82 (dd, J=8.1, 1.5 Hz, 1H), 7.56 (t, J=7.8 Hz, 1H), 7.38 (dd, J=7.7, 1.5 Hz, 1H), 7.14 (s, 1H), 4.49-4.37 (m, 2H), 4.31 (s, 1H), 3.71 (d, J=4.1 Hz, 1H), 3.65-3.43 (m, 1H), 3.18 (d, J=9.3 Hz, 1H), 3.02 (s, 1H), 2.91-2.74 (m, 2H), 2.70 (dd, J=13.6, 5.9 Hz, 2H), 2.55 (s, 3H), 1.81-1.60 (m, 1H), 1.38 (t, J=7.1 Hz, 3H), 1.34-1.06 (m, 4H), 0.92 (s, 9H).

### Step 14a. tert-Butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

The title compound can be alternatively prepared by the following method. A mixture of ethyl (R)-4-chloro-6-(2-cyanoethyl)-7-(2,3-dichlorophenyl)-8-fluoro-2-methylquinoline-3-carboxylate (40 g, 85 mmol), Li₂CO₃ (19 g, 258 mmol), and tert-butyl (1R,4R,5S)-5-amino-2-azabicyclo[2.1.1]hexane-2-carboxylate oxalate (29.4 g, 98 mmol) in DMSO (120 mL) was heated to 80° C. overnight. The reaction mixture was cooled to r.t. and MTBE (300 mL) and filtered. The solids were rinsed with MTBE (100 mL). The combined filtrate was washed with water (2×320 mL). The organic phase was separated. The solvent was removed under reduced pressure to give the product that was used for next step without further purification. An analytical sample was purified by silica gel column (0-10% MeOH in DCM). LCMS calc. for C₃₂H₃₃Cl₂FN₄O₄: 626.19. Found: 627 (M+H⁺). ¹H-NMR (400 MHz, DMSO-d₆) δ 8.09 (s, 1H), 7.82 (dd, J=8.1, 1.5 Hz, 1H), 7.56 (t, J=7.8 Hz, 1H), 7.38 (dd, J=7.7, 1.5 Hz, 1H), 7.14 (s, 1H), 4.49-4.37 (m, 2H), 4.31 (s, 1H), 3.71 (d, J==4.1 Hz, 1H), 3.65-3.43 (m, 1H), 3.18 (d, J==9.3 Hz, 1H), 3.02 (s, 1H), 2.91-2.74 (m, 2H), 2.70 (dd, J==13.6, 5.9 Hz, 2H), 2.55 (s, 3H), 1.81-1.60 (m, 1H), 1.38 (t, J=7.1 Hz, 3H), 1.34-1.06 (m, 4H), 0.92 (s, 6H).

The alternative atropisomer tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate is prepared by an analogous route by performing an analogous process starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 15. (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

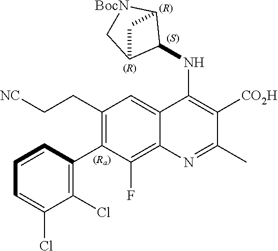

2 M NaOH (134 mL, 268 mmol) was added to a solution of tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (140.0 g, 223 mmol) in MeCN (560 ml) and MeOH (210 ml) at r.t. The mixture was heated to 50° C. for 1-1.5 h. The mixture was cooled to r.t. and acidified with 1M HCl to about pH 5. The MeCN and MeOH were removed under vacuum. The product was extracted by EtOAc (1.7 L). The aqueous phase was separated and extracted with EtOAc (420 mL). The combined EtOAc phases were concentrated under vacuum to give a residue. MTBE (300 mL) was added to the residue and the mixture slurry was agitated at r.t. for 2 h. The solids were isolated by filtration and the wet cake was washed with MTBE (2×100 mL). The solids were dried under vacuum at about 50° C. to give desired product (135 g, quantitative) that was used for next step without further purification.

### Step 15a. (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

The title compound can be alternatively prepared by the following process. Sodium trimethylsinolate (338 g, 95%) was added to a solution of tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (1400 g, 2.231 mol) in THF (14 L) and water (80 mL) at r.t. The mixture was heated to 50° C. for 1-3 h to complete the reaction. The mixture was cooled to r.t. and acidified with 1M HCl to about pH 5. The THF was removed under vacuum. The product was extracted by DCM (6 L). The aqueous phase was separated and extracted with DCM (6 L). The combined organic phases were concentrated under vacuum to give the product in DCM solution (6 L). The concentrated DCM solution was added to MTBE (7 L) was added to the residue and the mixture slurry was agitated at r.t. for 2 h. N-Heptane (7 L) was added to the mixture. The DCM was removed under vacuum. The solids were isolated by filtration and the wet cake was washed with n-heptane (2×3 L). The solids were dried under vacuum at about 50° C. to give desired product that was used for next step without further purification.

### Step 16. tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate

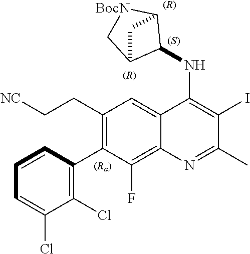

To a mixture of 4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid (132 g, 220 mmol), and sodium phosphate (74.4 g, 440 mmol) in anhydrous MeCN (1614 ml) was added N-iodosuccinimide (94 g, 396 mmol) and the mixture was stirred for 1 h. Water (1.6 L) was added to the mixture and resulting slurry was stirred for 5 h at r.t. The solids were isolated by filtration and the wet cake was reslurried in water (2.6 L) at r.t. for 5 h. The solids were isolated by filtration and the wet cake was washed with water (2×250 mL). The solids were dried under vacuum at about 50° C. to give desired product (120 g, 80% yield). LCMS calculated for C₃₉H₂₈Cl₂FIN₄O₂: 680.06. Found: 681 (M+H⁺). ¹H-NMR (400 MHz, DMSO-d₆) δ 7.94 (s, 1H), 7.82 (dd, J=8.0, 1.6 Hz, 1H), 7.56 (t, J=7.8 Hz, 1H), 7.50 (dd, J=7.7, 1.6 Hz, 1H), 5.49 (s, 1H), 4.28 (s, 2H), 3.09 (s, 1H), 2.96-2.58 (m, 8H), 1.71 (s, 1H), 1.59-0.96 (m, 11H).

### Step 17. tert-Butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate

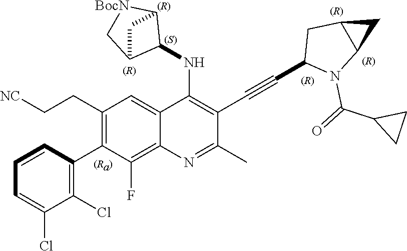

A mixture of cyclopropyl((1R,5R)-3-ethynyl-2-azabicyclo[3.1.0]hexan-2-yl)methanone (47.5 g, 260 mmol), tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate (136.5 g, 200 mmol), and tetrabutylammonium acetate (242 g, 801 mmol) in DMF (1100 ml) was subsurface purged with N₂ for 10 min. Tris(dibenzylideneacetone)dipalladium(0) (2.75 g, 3 mmol) was added to the mixture. The mixture was subsurface purged with N₂ for another 15 min before heating to 70° C. for 1 h. The reaction mixture was cooled to r.t. and added to half saturated NaHCO₃ (2200 mL). The solids were isolated by filtration and the wet cake was washed with water (600 mL). The solids were dried under vacuum at about 50° C. and purified by FCC eluted with 0-2% MeOH in EtOAc to give desired product (142 g, 96% yield). ¹H NMR (400 MHz, DMSO-d6) δ 8.03 (d, J=12.4 Hz, 1H), 7.81 (dd, J=8.1, 1.6 Hz, 1H), 7.55 (t, J=7.9 Hz, 1H), 7.36 (d, J=7.3 Hz, 1H), 6.70-6.44 (m, 1H), 5.68-5.13 (m, 1H), 4.54-4.18 (m, 2H), 4.00-3.80 (m, 1H), 3.51 (s, 1H), 3.19 (t, J=9.0 Hz, 1H), 3.07-2.91 (m, 1H), 2.78 (d, J=10.7 Hz, 3H), 2.66 (d, J=9.0 Hz, 3H), 2.57 (d, J=11.7 Hz, 4H), 2.36-2.08 (m, 2H), 1.88 (dd, J=17.9, 10.5 Hz, 2H), 1.35 (d, J=9.7 Hz, 2H), 1.15-0.59 (m, 16H).

### Step 17a. tert-Butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate

The title compound can alternatively be prepared by the following method. A mixture of cyclopropyl((1R,5R)-3-ethynyl-2-azabicyclo[3.1.0]hexan-2-yl)methanone (17.7 kg, 101 mol), tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate (64.7 kg, 95 mol), copper (1) iodide (0.42 kg 2 mol), tris (4-fluorophenyl)phosphine (0.39 kg, 1 mol) and K₂CO₃ (36.4 kg, 191 mol) in DMSO (488.4 L) was subsurface purged with N₂ for 30 min. Palladium (II) acetate (60 g, 30 mmol) was added to the mixture. The mixture was subsurface purged with N₂ for another 30 min. before heating to 50° C. for more than 10 h. The reaction mixture was cooled to r.t. and EtOAc (906 L) was added, followed by slow addition of water (1267 L) was added. The mixture was stirred at r.t. for 30 min. and filtered over a diatomaceous earth bed. The diatomaceous earth bed was rinsed with EtOAc (33 L). The organic phase was separated from the aqueous phase and the aqueous phase was back extracted with EtOAc (195 L). The combined organic phase was washed with water (195 L). To the EtOAc phase was added water (130 L) and ammonium pyrrolidinedithiocarbamate (3.1 kg, 19 mol). The mixture was agitated at 50° C. for no less than 4 h. The mixture was cooled to r.t. and polish filtered. The aqueous phase was separated and discarded. The organic phase was washed 30 with water (325 L). The organic phase was heated to 50° C. and passed through activated carbon cartridge. The solution is concentrated under vacuum and solvent swapped into toluene to remove residual water to give desired product in 98% solution yield. The toluene solution was solvent swap into NMP for next step indole-cyclization without further purification. ¹H NMR (400 MHz, DMSO-d₆) δ 8.03 (d, J=12.4 Hz, 1H), 7.81 (dd, J=8.1, 1.6 Hz, 1H), 7.55 (t, J=7.9 Hz, 1H), 7.36 (d, J=7.3 Hz, 1H), 6.70-6.44 (m, 1H), 5.68-5.13 (m, 1H), 4.54-4.18 (m, 2H), 4.00-3.80 (m, 1H), 3.51 (s, 1H), 3.19 (t, J=9.0 Hz, 1H), 3.07-2.91 (m, 1H), 2.78 (d, J=10.7 Hz, 3H), 2.66 (d, J=9.0 Hz, 3H), 2.57 (d, J=11.7 Hz, 4H), 2.36-2.08 (m, 2H), 1.88 (dd, J=17.9, 10.5 Hz, 2H), 1.35 (d, J=9.7 Hz, 2H), 1.15-0.59 (m, 16H).

The alternative atropisomer tert-butyl (1R,4R,5S)-5-(((Sₐ)-6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate is prepared by an analogous route by performing processes analogous to Steps 14-17 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 18. tert-Butyl (1R,4R,5S)-5-((Rₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate

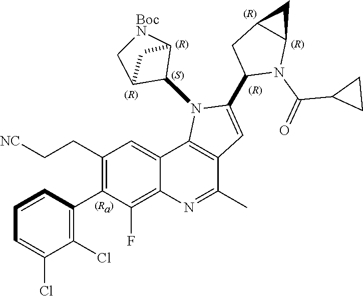

To a mixture of tert-butyl (1R,4R,5S)-5-((6-(2-cyanoethyl)-3-(((1R,3R,5R)-2-(cyclopropanecarbonyl)-2-azabicyclo[3.1.0]hexan-3-yl)ethynyl)-7-(2,3-dichlorophenyl)-8-fluoro-2-methylquinolin-4-yl)amino)-2-azabicyclo[2.1.1]hexane-2-carboxylate (141.0 g, 159 mmol) and Cs₂CO₃ (78 g, 238 mmol) in DMSO (1 L) or N-methy-2-pyrrolidone was heated to 80-85° C. for 90 min. The reaction was cooled to r.t. and water (2 L) was gradually added. The product was gradually precipitated out of the solution. The resulting slurry was stirred at r.t. for 1 h. The solids were isolated by filtration and the wet cake was washed with water (2×300 mL). The wet solids were dried under vacuum. The solids were purified by FCC with 60-100% EtOAc in DCM. The solvents were removed and the solids in heptane (840 mL) were crystallized from EtOAc (420 mL) and MTBE (420 mL) and heptane 9840 mL) to give desired product (122 g, 87% yield). LCMS calculated for C₄₀H₄₀Cl₂FN₅O₃: 727.25. Found: 728 (M+H⁺). ¹H NMR (500 MHz, DMSO-d6) δ 8.12 (s, 1H), 7.81 (dt, J=8.0, 2.1 Hz, 1H), 7.55 (td, J=7.8, 5.0 Hz, 1H), 7.45-7.29 (m, 1H), 6.26 (s, 1H), 5.81-5.49 (m, 1H), 5.34-5.13 (m, 1H), 5.00 (dd, J=14.3, 6.8 Hz, 1H), 4.19-3.97 (m, 1H), 3.63 (dt, J=6.8, 3.1 Hz, 1H), 3.40 (d, J=9.4 Hz, 1H), 3.27-3.09 (m, 1H), 2.95 (dt, J=14.2, 7.6 Hz, 1H), 2.89-2.73 (m, 3H), 2.70 (d, J=2.7 Hz, 4H), 2.34-2.20 (m, 1H), 2.21-1.97 (m, 2H), 1.73 (dp, J=15.0, 4.8 Hz, 1H), 1.66-1.34 (m, 2H), 1.21-1.03 (m, 1H), 1.02-0.79 (m, 4H), 0.78-0.22 (m, 11H).

### Step 19. 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile

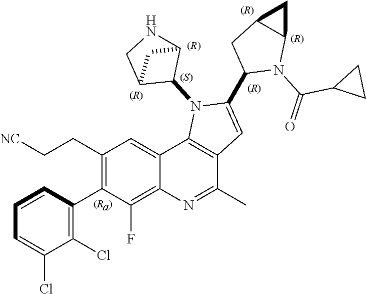

To a solution of tert-Butyl (1R,4R,5S)-5-((Rₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate (167.7 g, 230.1 mmol) in DCM (1.35 L) was added TMSI (69 g, 345 mmol) at r.t. and stirred for 1 h. NaHCO₃ (500 mL) was added to quench the reaction. The organic phase was isolated and washed with water. The solvent was evaporated by rotavapor and the residue was passed over silica gel bed (1-20% MeOH in DCM). The solvent was swapped into EtOAc and MTBE to give crystalline product (136 g, 94% yield). LCMS calculated for C₃₅H₃₂Cl₂FN₅O: 627.20. Found: 628 (M+H⁺). ¹H-NMR (400 MHz, DMSO-d₆) 5 ¹H NMR (500 MHz, DMSO-d6) δ 8.15 (d, J=13.6 Hz, 1H), 7.89-7.73 (m, 1H), 7.64-7.33 (m, 2H), 6.69-6.14 (m, 1H), 5.76-5.43 (m, 1H), 4.97 (d, J=4.9 Hz, 1H), 4.31 (dd, J=17.0, 6.0 Hz, 1H), 4.18-3.94 (m, 1H), 3.58-3.45 (m, 1H), 2.94 (dt, 2H, J=12.4, 6.1 Hz), 2.89-2.56 (m, 8H), 2.44-2.19 (m, 2H), 2.07 (d, J=12.9 Hz, 1H), 1.96-1.54 (m, 3H), 1.30-1.13 (m, 1H), 1.06-0.20 (m, 6H).

The alternative atropisomer 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile is prepared by an analogous route by performing processes analogous to Steps 14-19 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 20: 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile monohydrochloride dihydrate (Compound 1)

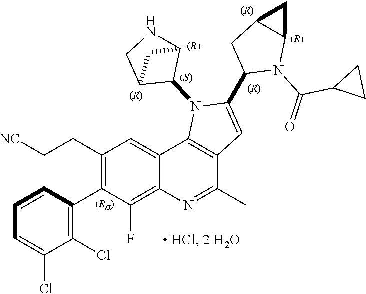

To a solution of dissolved 3-((Rₐ)-1-((1R,4R,5S)-2-azabicyclo[2.1.1]-hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile free base (53.8 g, 85 mmol) in MeOH (110 mL), EtOAc (50 mL), water (11 mL) and MTBE (110 mL) was added 6N HCl (14.5 mL) at 30-50° C. The mixture was seeded and the solution gradually turned cloudy. MTBE (440 mL) was slowly added to the mixture at about 40° C. over 1 h. The mixture was cooled to about 15° C. and agitated for 2 h. The solids were isolated by filtration and the wet cake was washed with 5% MeOH and 20% EtOAc in MTBE (2×110 mL). The wet solids were slurried in EtOAc (270 mL) and dried under vacuum at about 50° C. to give desired product (53.7 g, 90% yield). LCMS calculated for C₃₅H₃₂Cl₂FN₅O: 627.20. Found: 628 (M+H⁺). ¹H NMR (500 MHz, DMSO-d₆) δ 8.15 (s, 1H), 7.83 (dd, J=8.1, 1.6 Hz, 1H); 7.57 (dd, J=7.9, 7.9, 1H); 7.45 (dd, J=7.7, 1.6 Hz, 1H); 6.44 (s, 1H); 5.65 (s, 1H); 5.51 (d, J=10.6 Hz, 1H); 4.14 (td, J=6.4, 2.6 Hz, 1H); 3.84-3.90 (m, 1H); 3.30-3.37 (m, 1H); 3.43-3.50 (m, 1H); 2.86-2.95 (m, 1H); 2.83-2.92 (m, 1H); 2.79 (s, 3H); 2.70-2.79 (m, 1H); 2.29-2.35 (m, 1H); 2.25-2.32 (m, 1H); 1.97 (dd, J=13.0, 2.6 Hz, 1H); 1.69-1.83 (m, 1H); 1.65 (d, J=9.1 Hz, 1H); 0.91-1.00 (m, 2H); 0.82-0.88 (m, 2H); 0.72-0.80 (m, 1H); 0.63-0.69 (m, 1H). 13C NMR (125 MHz, DMSO-d₆) δ 171.6; 145.8; 132.8; 135.1; 132.8; 131.9; 131.5; 131.4; 129.2; 101.6; 120.7; 57.9; 56.5; 44.5; 42.5; 30.5; 38.3; 32.8; 22.1; 17.5; 17.1; 13.2; 13.0; 7.70; 7.80. 19F NMR (376 MHz, DMSO-d₆) δ −122.1 (s).

The alternative atropisomer 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile monohydrochloride dihydrate (Compound 1b) is prepared by an analogous route by performing processes analogous to Steps 15-21 starting from 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile instead of 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

### Example 2: Synthesis Procedure for Synthetic Intermediates

### Intermediate 1. Ethyl (R)-4-chloro-6-(2-cyanoethyl)-7-(2,3-dichlorophenyl)-8-fluoro-2-methylquinoline-3-carboxylate

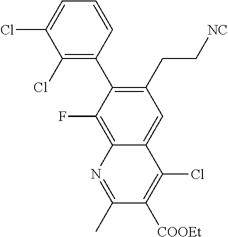

### Step 1. 3-Amino-2′,3′-dichloro-2-fluoro-[1,1′-biphenyl]-4-carboxylic acid

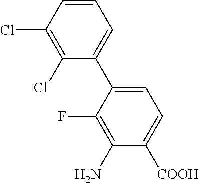 [SMILES: Nc1c(C(=O)O)ccc(-c2cccc(Cl)c2Cl)c1F]

A mixture of 2-amino-4-bromo-3-fluorobenzoic acid (28.0 g, 120 mmol), (2,3-dichlorophenyl)boronic acid (25.1 g, 132 mmol), bis(di-tert-butyl(4-dimethylaminophenyl)phosphine)dichloropalladium(II) (2.12 g, 3.00 mmol) and potassium phosphate (50.8 g, 239 mmol) in 1,4-dioxane (170 mL) and water (30 mL) was sparged with N₂ and heated at 70° C. for 1 h. Once completed, the reaction mixture was cooled down to r.t. and poured into 1 N HCl (200 mL). The mixture was stirred for another 10 min, resulting in precipitation. The solids were collected on a fritted filter, washed with water followed by hexanes and dried under reduced pressure to afford the sub-title compound in near quantitative yield. The crude product was used in next step without further purification. LC-MS calc. for C₁₃H₉Cl₂FNO₂ (M+H)⁺: m/z=300.0. found 300.0.

### Step 2. 3-Amino-6-bromo-2′,3′-dichloro-2-fluoro-[1,1′-biphenyl]-4-carboxylic acid

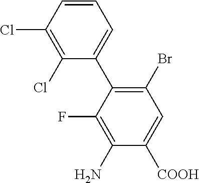 [SMILES: Nc1c(C(=O)O)cc(Br)c(-c2cccc(Cl)c2Cl)c1F]

To a solution of 3-amino-2′,3′-dichloro-2-fluoro-[1,1′-biphenyl]-4-carboxylic acid (35.8 g, 119 mmol) in DMSO (100 mL) was added NBS (22.3 g, 125 mmol). The resulting mixture was heated at 50° C. for 1 h. Once completed, the reaction mixture was cooled down to r.t. and poured into ice water (400 mL). To the suspension, was added 20 mL saturated Na₂S₂O₃ solution. After stirring for 15 min, the solids were collected on a fritted filter, washed with water followed by hexanes and dried under reduced pressure to afford the sub-title compound (43.0 g, 95% yield). The crude product was used in next step without further purification. LC-MS calc. for C₁₃H₈BrCl₂FNO₂ (M+H)+: m/z=377.9, 379.9. found 378.0, 380.0.

### Step 3. 6-Bromo-7-(2,3-dichlorophenyl)-8-fluoro-2H-benzo[d][1,3]oxazine-2,4(1H)-dione

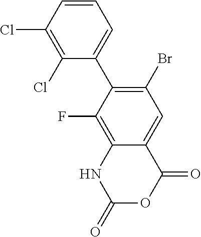 [SMILES: O=c1[nH]c2c(F)c(-c3cccc(Cl)c3Cl)c(Br)cc2c(=O)o1]

To a solution of 3-amino-6-bromo-2′,3′-dichloro-2-fluoro-[1,1′-biphenyl]-4-carboxylic acid (38.6 g, 102 mmol) in THF (300 mL) was added triphosgene (10.6 g, 35.6 mmol) portion-wise. After addition, the mixture was heated at 60° C. for 0.5 h. Once completed, the reaction mixture was cooled down to r.t. and poured into heptane (1000 mL). After stirring for 1 h, the solids were collected on a fritted filter, washed with hexanes and dried under reduced pressure to afford the sub-title compound in near quantitative yield. The crude product was used in next step without further purification.

### Step 4. Ethyl 6-bromo-7-(2,3-dichlorophenyl)-8-fluoro-4-hydroxy-2-methylquinoline-3-carboxylate

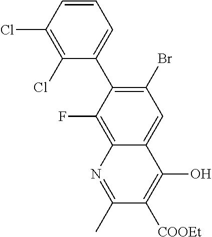 [SMILES: CCOC(=O)c1c(C)nc2c(F)c(-c3cccc(Cl)c3Cl)c(Br)cc2c1O]

To a solution of 6-bromo-7-(2,3-dichlorophenyl)-8-fluoro-2H-benzo[d][1,3]oxazine-2,4(1H)-dione (41.5 g, 102 mmol) in DMSO (200 mL), was added (1-ethoxy-1,3-dioxobutan-2-yl)sodium (18.7 g, 123 mmol) portion-wise. After addition, the mixture was heated at 80° C. for 1 h. Once completed, the reaction mixture was cooled down to r.t. and poured into 1 N HCl (400 mL). After stirring for 1 h, the solids were collected on a fritted filter, washed with water followed by hexanes and dried under reduced pressure to afford the sub-title compound (40.0 g, 83% yield). The crude product was used in next step without further purification. LC-MS calc. for C₁₉H₁₄BrCl₂FNO₃ (M+H)⁺: m/z=471.9, 473.9. found 471.9, 474.0.

### Step 5. Ethyl (E)-6-(2-cyanovinyl)-7-(2,3-dichlorophenyl)-8-fluoro-4-hydroxy-2-methylquinoline-3-carboxylate

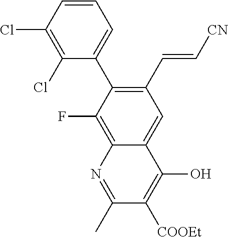 [SMILES: CCOC(=O)c1c(C)nc2c(F)c(-c3cccc(Cl)c3Cl)c(/C=C/C#N)cc2c1O]

To a solution of ethyl 6-bromo-7-(2,3-dichlorophenyl)-8-fluoro-4-hydroxy-2-methylquinoline-3-carboxylate (35.0 g, 74.0 mmol), bis(di-tert-butyl(4-dimethylaminophenyl)phosphine)dichloropalladium(II) (2.62 g, 3.70 mmol) in DMF (100 mL), acrylonitrile (12.3 mL, 185 mmol) and NEt₃ (30.9 mL) were added. The mixture was sparged with N₂ and heated at 85° C. for 1 h. Once completed, the reaction mixture was cooled down to r.t. and poured into 1 N HCl (500 mL). After stirring for 1 h, the solids were collected on a fritted filter, washed with water followed by hexanes and dried under reduced pressure to afford the sub-title compound (19.2 g, 58% yield). The crude product was used in next step without further purification. LC-MS calc. for C₂₂H₁₆Cl₂FN₂O₃(M+H)⁺: m/z=445.0. found 445.0.

### Step 6. Ethyl (E)-4-chloro-6-(2-cyanovinyl)-7-(2,3-dichlorophenyl)-8-fluoro-2-methylquinoline-3-carboxylate

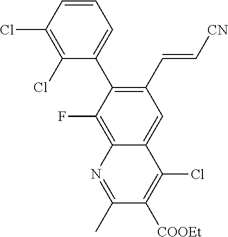 [SMILES: CCOC(=O)c1c(C)nc2c(F)c(-c3cccc(Cl)c3Cl)c(/C=C/C#N)cc2c1Cl]

To a slurry of ethyl (E)-6-(2-cyanovinyl)-7-(2,3-dichlorophenyl)-8-fluoro-4-hydroxy-2-methylquinoline-3-carboxylate (30.0 g, 67.4 mmol) and benzyltriethylammonium chloride (15.4 g, 67.4 mmol) in MeCN (150 mL) at 0° C., was added DIPEA (23.5 mL, 135 mmol). Upon stirring at 0° C., phosphoryl chloride (12.6 mL, 135 mmol) was added dropwise into the mixture. Then the mixture was heated at 60° C. for 1 h. Upon completion, the reaction mixture was cooled to r.t. and slowly poured into ice water (1000 mL). The mixture was extracted with DCM three times, dried over Na₂SO₄, filtered and concentrated. The crude product was further purified by FCC (0-50% EtOAc/hexanes) to afford the sub-title compound (4.5 g, 14% yield). LC-MS calc. for C₂₂H₁₅Cl₃FN₂O₂(M+H)⁺: m/z=463.0. found 463.0.

### Step 7. Ethyl (R)-4-chloro-6-(2-cyanoethyl)-7-(2,3-dichlorophenyl)-8-fluoro-2-methylquinoline-3-carboxylate

A mixture of copper(II) acetate monohydrate (0.19 g, 0.97 mmol) and Xantphos (0.56 g, 0.97 mmol) was stirred in toluene (1 mL) and tert-butanol (9 mL) at 60° C. for 0.5 h to afford a homogeneous solution. In a separate vial, to a mixture of ethyl (E)-4-chloro-6-(2-cyanovinyl)-7-(2,3-dichlorophenyl)-8-fluoro-2-methylquinoline-3-carboxylate (4.5 g, 9.70 mmol) and polymethylhydrosiloxane (3.5 g, 58.2 mmol) in toluene (12 mL) at 60° C., was added the previous copper-containing solution. The mixture was stirred at 60° C. for 0.5 h. Upon completion, the reaction mixture was filtered through diatomaceous earth and concentrated. The crude product was purified using FCC (0-40% EtOAc/DCM) to afford a mixture of two atropisomers (2.0 g, 44% yield). The title compound was separated from its atropisomer using chiral supercritical fluid chromatography (ChiralPak IJ column, eluting with 40% MeOH in CO₂ at a flow rate of 70 mL/min; the title compound eluted after its atropisomer). LC-MS calc. for C₂₂H₁₇Cl₃FN₂O₂(M+H)⁺: m/z=465.0. found 465.0.

### Intermediate 2. tert-Butyl (1R,4R,5S)-5-(((R)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate

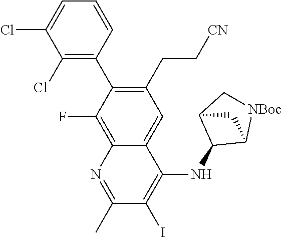 [SMILES: Cc1nc2c(F)c(-c3cccc(Cl)c3Cl)c(CCC#N)cc2c(N[C@H]2[C@@H]3C[C@H]2N(C(=O)OC(C)(C)C)C3)c1I]

### Step 1. tert-Butyl (1R,4R,5S)-5-(((R)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

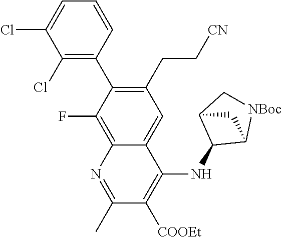 [SMILES: CCOC(=O)c1c(C)nc2c(F)c(-c3cccc(Cl)c3Cl)c(CCC#N)cc2c1N[C@H]1[C@@H]2C[C@H]1N(C(=O)OC(C)(C)C)C2]

To a solution of ethyl (R)-4-chloro-6-(2-cyanoethyl)-7-(2,3-dichlorophenyl)-8-fluoro-2-methylquinoline-3-carboxylate (intermediate 1, 7.2 g, 15.5 mmol) in N-methyl-2-pyrrolidone (21 mL), was added tert-butyl (1R,4R,5S)-5-amino-2-azabicyclo[2.1.1]hexane-2-carboxylate (5.52 g, 27.8 mmol) and DIPEA (8.1 mL). The resulting mixture was heated at 80° C. for 18 h. Once completed, the reaction mixture was cooled down to r.t. and poured into 1 N HCl (300 mL) and ice mixture. After stirring for 0.5 h, the solids were collected on a fritted filter, washed with water followed by hexanes and dried under reduced pressure to afford white solids (8.2 g, 85% yield). The crude product was used in next step without further purification. LC-MS calc. for C₃₂H₃₄Cl₂FN₄O₄(M+H)⁺: m/z=627.2. found 627.1.

### Step 2. (R)-4-(((1R,4R,5S)-2-(tert-Butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

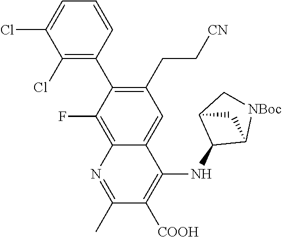 [SMILES: Cc1nc2c(F)c(-c3cccc(Cl)c3Cl)c(CCC#N)cc2c(N[C@H]2[C@@H]3C[C@H]2N(C(=O)OC(C)(C)C)C3)c1C(=O)O]

To a solution of tert-butyl (1R,4R,5S)-5-(((R)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (4.0 g, 6.37 mmol) in MeCN (13 mL), was added 1 N NaOH (16 mL). The mixture was heated at 50° C. for 2 h. Once completed, the reaction mixture was cooled down to r.t. and acidified to pH 5 using 1 N HCl. The organic volatiles were removed under reduced pressure. The residue aqueous phase was extracted with EtOAc three times. The combined organic layers were dried over Na₂SO₄, filtered and concentrated under reduced pressure to give a yellow solid (3.70 g, 97% yield). The crude material was used in the next step without further purification. LC-MS calc. for C₃₀H₃₀Cl₂FN₄O₄(M+H)⁺: m/z=599.2. found 599.1.

### Step 3. tert-Butyl (1R,4R,5S)-5-(((R)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate

To a solution of (R)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid (3.70 g, 6.17 mmol) in MeCN (6.2 mL), was added potassium phosphate (2.62 g, 12.34 mmol) and NIS (2.50 g, 11.1 mmol). The mixture was stirred at r.t. for 1 h. Once completed, the reaction mixture was poured into saturated Na₂S₂O₃ solution. After stirring for 10 min, the mixture was extracted with EtOAc three times. The combined organic layers were dried over Na₂SO₄, filtered and concentrated under reduced pressure. The crude product was further purified with FCC (0-100% EtOAc/hexanes) to afford the title compound as an off-white solid (1.95 g, 46% yield). LC-MS calc. for C₂₉H₂₉Cl₂FIN₄O₂(M+H)⁺: m/z=681.1. found 681.0.

### Intermediate 3. Methyl (1R,3R,4S)-2-((S)-1-phenylethyl)-2-azabicyclo[2.2.1]hept-5-ene-3-carboxylate

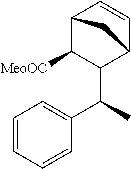

### Step 1. Methyl 2-hydroxy-2-methoxyacetate

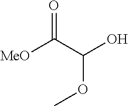

A solution of glyoxylic acid monohydrate (41.4 g, 450 mmol) in anhydrous MeOH (200 mL) was heated to 70° C. overnight. After cooling to r.t., the mixture was stirred with solid NaHCO₃ for 10 min. The resulting mixture was filtered and concentrated under reduced pressure to afford an oily residue. The residue was dissolved in CH₂Cl₂, dried over Na₂SO₄, filtered and concentrated to afford the product (40.0 g, 82% yield). The product was used in next step without further purification.

### Step 2. Methyl (S,E)-2-((1-phenylethyl)imino)acetate

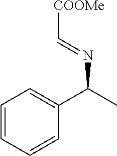

To a solution of methyl 2-hydroxy-2-methoxyacetate (40.0 g, 333 mmol) in toluene (95 mL) was added (S)-1-phenylethan-1-amine (40.4 g, 333 mmol) slowly. The mixture was stirred for 1 h at r.t. and diluted with EtOAc. The organic layer was washed with brine, dried over Na₂SO₄, filtered and concentrated under reduced pressure to a yellow oil. The crude product was used in the next step without further purification.

### Step 3. Methyl (1R,3R,4S)-2-((S)-1-phenylethyl)-2-azabicyclo[2.2.1]hept-5-ene-3-carboxylate

To a solution of methyl (S,E)-2-((1-phenylethyl)imino)acetate (63.7 g, 333 mmol) in 2,2,2-trifluoroethanol (800 mL) at −10° C., was added TFA (25.5 mL, 333 mmol). The reaction mixture was allowed to stir at −10° C. for 1 h before cyclopentadiene (24.2 g, 366 mmol) was added slowly. The mixture was stirred at −10° C. for another 0.5 h and then allowed to warm up to r.t. After removal of volatiles, the residue was diluted with 2 N HCl (500 mL) and washed with Et₂O. The organic layer was extracted with 2 N HCl (100 mL). The combined aqueous layer was neutralized with 28% NH₄OH and extracted with EtOAc three times. The combined organic layers were dried over Na₂SO₄, filtered and concentrated. The crude product was purified by FCC (0-10% EtOAc/hexanes) in batches to afford the title compound as a colorless solid. LC-MS calc. for C₁₆H₂₀NO₂ (M+H)⁺: m/z=258.1. found 258.2. ¹H NMR (500 MHz, CDCl₃) δ 7.32-7.27 (m, 2H), 7.25 (m, 2H), 7.22-7.16 (m, 1H), 6.44 (ddd, J=5.7, 3.1, 1.2 Hz, 1H), 6.29 (dd, J=5.7, 2.0 Hz, 1H), 4.33 (h, J=1.5 Hz, 1H), 3.37 (s, 3H), 3.06 (q, J=6.5 Hz, 1H), 2.93 (dq, J=3.3, 1.6 Hz, 1H), 2.24 (d, J=0.9 Hz, 1H), 2.13 (dt, J=8.4, 1.7 Hz, 1H), 1.48-1.41 (m, 4H).

### Intermediate 4. 2-(tert-butyl) 3-methyl (1R,3R,4R,5S)-5-hydroxy-2-azabicyclo[2.2.1]heptane-2,3-dicarboxylate

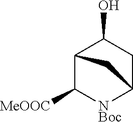 [SMILES: COC(=O)[C@H]1[C@H]2C[C@H](C[C@@H]2O)N1C(=O)OC(C)(C)C]

### Step 1. Methyl (1R,3R,4R,5S)-5-hydroxy-2-((S)-1-phenylethyl)-2-azabicyclo[2.2.1]heptane-3-carboxylate

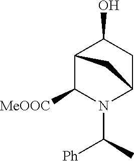 [SMILES: COC(=O)[C@H]1[C@H]2C[C@H](C[C@@H]2O)N1[C@@H](C)c1ccccc1]

To a solution of methyl (1R,3R,4S)-2-((S)-1-phenylethyl)-2-azabicyclo[2.2.1]hept-5-ene-3-carboxylate (intermediate 3, 5.3 g, 20.6 mmol) in THF (70 mL) at 0° C., was added a 0.5 N THF solution of 9-BBN (51.5 mL, 25.7 mmol). The reaction mixture was allowed to warm up to r.t. and stirred for 18 h. Then the reaction mixture was cooled to 0° C. and a 2 N NaOH solution (36.0 mL, 72.1 mmol) was added followed by 30% H₂O₂ (10.5 mL). The reaction mixture was allowed to warm up to r.t. and stirred for 1 h. The reaction mixture was diluted with EtOAc, washed with brine, dried over Na₂SO₄, filtered and concentrated. The crude product was purified by FCC (50%-70% EtOAc/hexanes) to afford the sub-title compound (2.0 g, 38% yield). LC-MS calc. for C₁₆H₂₂NO₃ (M+H)⁺: m/z=276.2. found 276.2.

### Step 2. Methyl (1R,3R,4R,5S)-5-hydroxy-2-azabicyclo[2.2.1]heptane-3-carboxylate

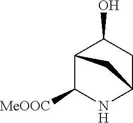 [SMILES: COC(=O)[C@@H]1N[C@@H]2C[C@H]1[C@@H](O)C2]

To a solution of methyl (1R,3R,4R,5S)-5-hydroxy-2-((S)-1-phenylethyl)-2-azabicyclo[2.2.1]heptane-3-carboxylate (2.00 g, 7.26 mmol) in EtOH (35 mL) was added 20% Pd(OH)₂/C (0.58 g). The mixture was stirred under H₂ for 18 h. The resulting mixture was filtered through diatomaceous earth and concentrated to afford the sub-title compound. The crude material was used for next step without further purification. LC-MS calc. for C₃H₁₄NO₃ (M+H)⁺: m/z=172.1. found 172.1.

### Step 3. 2-(tert-Butyl) 3-methyl (1R,3R,4R,5S)-5-hydroxy-2-azabicyclo[2.2.1]heptane-2,3-dicarboxylate

To methyl (1R,3R,4R,5S)-5-hydroxy-2-azabicyclo[2.2.1]heptane-3-carboxylate (1.24 g, 7.26 mmol) dissolved in THF (10 mL), was added DIPEA (5.10 mL, 29.1 mmol) and Boc₂O (3.96 g, 18.2 mmol). The reaction mixture was stirred at r.t. for 0.5 h and diluted with EtOAc. After washing with 0.01 N HCl and brine, the organic fraction was dried over Na₂SO₄, filtered and concentrated. The crude product was further purified by FCC (0%-100% EtOAc/hexanes) to afford the title compound (1.88 g, 95% yield). LC-MS calc. for C₉H₁₄NO₅ (M-^{t}Bu+2H)⁺: m/z=216.1. found 216.1.

### Intermediate 5. tert-butyl (1R,3R,4R,5S)-5-((tert-butyldiphenylsilyl)oxy)-3-ethynyl-2-azabicyclo[2.2.1]heptane-2-carboxylate

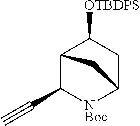

### Step 1. 2-(tert-Butyl) 3-methyl (1R,3R,4R,5S)-5-((tert-butyldiphenylsilyl)oxy)-2-azabicyclo[2.2.1]heptane-2,3-dicarboxylate

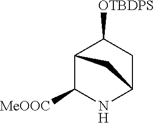

To a solution of 2-(tert-butyl) 3-methyl (1R,3R,4R,5S)-5-hydroxy-2-azabicyclo[2.2.1]heptane-2,3-dicarboxylate (intermediate 4, 1.88 g, 6.92 mmol) in DMF (140 mL), was added tert-butylchlorodiphenylsilane (2.08 g, 7.64 mmol) and imidazole (1.40 g, 20.8 mmol). The reaction mixture was stirred at r.t. for 18 h. The mixture was diluted 20 with EtOAc, washed with brine five times, dried over Na₂SO₄, filtered and concentrated. The crude product was purified by FCC (0%-40% EtOAc/hexanes) to afford the sub-title compound. LC-MS calc. for C₂₅H₃₂NO₅Si (M-^{t}Bu+2H)⁺: m/z=454.2. found 454.2.

### Step 2. tert-Butyl (1R,3R,4R,5S)-5-((tert-butyldiphenylsilyl)oxy)-3-(hydroxymethyl)-2-azabicyclo[2.2.1]heptane-2-carboxylate

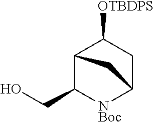

To a solution of 2-(tert-butyl) 3-methyl (1R,3R,4R,5S)-5-((tert-butyldiphenylsilyl)oxy)-2-azabicyclo[2.2.1]heptane-2,3-dicarboxylate (1.53 g, 3.01 mmol) in THF (15 mL), was added a 2 N THF solution of LiBH₄ (3.8 mL, 7.52 mmol). The mixture was stirred at r.t. for 8 h and then quenched by slow addition of a saturated NH₄Cl solution. The mixture was diluted with EtOAc, washed with brine, dried over Na₂SO₄, filtered and concentrated. The crude product was purified by FCC (0-60% EtOAc/hexanes) to afford the sub-title compound (1.39 g, 96% yield). LC-MS calc. for C₂₄H₃₂NO₄Si (M-^{t}Bu+2H)⁺: m/z=426.2. found 426.2.

### Step 3. tert-Butyl (1R,3R,4R,5S)-5-((tert-butyldiphenylsilyl)oxy)-3-formyl-2-azabicyclo[2.2.1]heptane-2-carboxylate

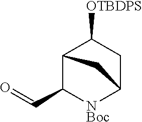

To a solution of oxalyl chloride (0.73 g, 5.76 mmol) in DCM (5.3 mL) cooled to −78° C., was added DMSO (0.61 mL, 8.64 mmol) slowly. After stirring for 10 min., a DCM (1 mL) solution of tert-butyl (1R,3R,4R,5S)-5-((tert-butyldiphenylsilyl)oxy)-3-(hydroxymethyl)-2-azabicyclo[2.2.1]heptane-2-carboxylate (1.39 g, 2.88 mmol) was added. The reaction mixture was stirred at −78° C. for 1 h before DIPEA (1.5 mL) was added. The reaction mixture was allowed to warm up to r.t. and stirred for another 0.5 h. Then the reaction mixture was poured into a DCM (15 mL)/28% NH₄OH (1.5 mL) mixture. After stirring for 10 min., the mixture was diluted with water. The organic phase was separated, dried over Na₂SO₄, filtered and concentrated to give crude product, which was used in the next step without further purification. LC-MS calc. for C₂₄H₃₀NO₄Si (M-^{t}Bu+2H)⁺: m/z=424.2. found 424.3.

### Step 4. tert-Butyl (1R,3R,4R,5S)-5-((tert-butyldiphenylsilyl)oxy)-3-ethynyl-2-azabicyclo[2.2.1]heptane-2-carboxylate

To a solution of tert-butyl (1R,3R,4R,5S)-5-((tert-butyldiphenylsilyl)oxy)-3-formyl-2-azabicyclo[2.2.1]heptane-2-carboxylate (1.38 g, 2.88 mmol) in MeOH (15 mL) was added dimethyl(1-diazo-2-oxopropyl)phosphonate (0.61 g, 3.17 mmol) and K₂CO₃ (1.19 g, 8.64 mmol). After stirring for 18 h, the reaction mixture was filtered through diatomaceous earth. The filtrate was concentrated. The residue was extracted with EtOAc, filtered through diatomaceous earth and concentrated. The crude product was purified by FCC (0-40% EtOAc/hexanes) to afford the title compound (0.20 g, 51% over 2 steps). LC-MS calc. for C₂₅H₃₀NO₃Si (M-^{t}Bu+2H)⁺: m/z=420.2. found 420.2.

### Example 3. Synthesis of cyclopropyl((1R,3R,5R)-3-ethynyl-2-azabicyclo[3.1.0]hexan-2-yl)methanone (step 17 in Example 1)

### Step 1. 1-(tert-Butyl) 2-ethyl (R)-2,3-dihydro-1H-pyrrole-1,2-dicarboxylate

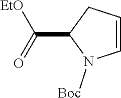

To a solution of 1-(tert-butyl) 2-ethyl (R)-5-oxopyrrolidine-1,2-dicarboxylate (241 g, 0.938 mol) in anhydrous toluene (1.6 L) was added 1M lithium triethyl borohydride in THF (1.01 L, 1.01 mol) dropwise at −50-−40° C. over 1 h. After addition, the mixture was stirred for 1 h at about −50° C. DIPEA (726 mL, 4.17 mol) was added to the mixture dropwise over 1 h. DMAP (1.49 g, 12.2 mmol) was added to the mixture, followed by the dropwise addition of trifluoroacetic anhydride (156.5 mL, 1.126 mol) over 1.5 h. After addition, the mixture was stirred for 1 h at about −50° C., then slowly warmed to r.t. The mixture was stirred for 1 h at r.t. The reaction mixture was cooled to 0° C. and diluted slowly with water (2.41 L), while maintaining the temperature below 10° C. during addition. The organic layer was separated and washed with water (2.41 L) and saturated brine (720 mL). The organic layer was dried over sodium sulfate (120 g). The solution was concentrated under reduced pressure to give desired product (230 g, quant.) as yellow oil. GCMS calc. for C₁₂H₁₉NO₄: 241.1. Found: 214.2 (M+). ¹H-NMR (400 MHz, CDCl₃) δ 6.70-6.48 (m, 1H), 4.99-4.86 (m, 1H), 4.70-4.52 (m, 1H), 4.30-4.11 (m, 2H), 3.15-2.98 (m, 1H), 2.73-2.57 (m, 1H), 1.53-1.38 (m, 9H), 1.34-1.21 (m, 4H).

### Step 2. 2-(tert-Butyl) 3-ethyl (1R,3R,5R)-2-azabicyclo[3.1.0]hexane-2,3-dicarboxylate

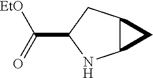

To a solution 1-(tert-butyl) 2-ethyl (R)-2,3-dihydro-1H-pyrrole-1,2-dicarboxylate (230 g, 0.938 mol) in toluene (2.3 L) was added 1.1M diethylzinc in toluene (1.7 L, 1.87 mol) at −30 to −25° C. over 1 h. Chloroiodomethane (273 mL, 3.752 mol) was added to the mixture dropwise over 2 h at about −30 to −20° C. and the mixture was stirred for 16 h. Half-saturated sodium bicarbonate (2.3 L) was added to the mixture and the mixture was warmed up to r.t. The mixture was filtered over diatomaceous earth to remove white solids and the filter bed was rinsed with toluene (1.5 L). The organic layer was separated from the filtrate and washed with water (2×1.15 L) and saturated brine (1.15 L). The toluene solution was concentrated under reduced pressure to give a 6 to 1 mixture (231 g) of 2-(tert-Butyl) 3-ethyl (1R,3R,5R)-2-azabicyclo[3.1.0]hexane-2,3-dicarboxylate and 2-(tert-butyl) 3-ethyl (1 S,3R,5S)-2-azabicyclo[3.1.0]hexane-2,3-dicarboxylate as yellow oil as determined by GCMS analysis.

Aqueous methyl amine (40%, 344 g) was added to a crude mixture product obtained above (226 g) and the mixture was stirred for 16 h at r.t. Water (340 mL) and MTBE (340 mL) was added to the mixture. The organic layer was separated and washed with water (340 mL) and saturated brine (230 mL). The solution was concentrated under reduced pressure to give 2-(tert-butyl) 3-ethyl (1R,3R,5R)-2-azabicyclo[3.1.0]hexane-2,3-dicarboxylate (177 g, 73% calc. yield) as yellow oil, which contained 2% 2-(tert-butyl) 3-ethyl (1 S,3R,5S)-2-azabicyclo[3.1.0]hexane-2,3-dicarboxylate as determined by GCMS analysis. GCMS calc. for C₁₃H₂₁NO₄: 255.1. Found: 255.1 (M+). ¹H-NMR (400 MHz, CDCl₃) δ 4.56-4.39 (m, 1H), 4.18-4.01 (m, 2H), 3.51-3.36 (m, 1H), 2.60-2.42 (m, 1H), 2.00-1.92 (m, 1H), 1.45-1.32 (m, 9H), 1.23-1.15 (m, 4H), 0.87-0.79 (m, 1H), 0.70-0.56 (m, 1H).

### Step 3. tert-Butyl (1R,3R,5R)-3-(hydroxymethyl)-2-azabicyclo[3.1.0]hexane-2-carboxylate

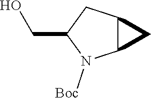

A solution of 2-(tert-butyl) 3-ethyl (1R,3R,5R)-2-azabicyclo[3.1.0]hexane-2,3-dicarboxylate (177 g, 0.694 mol) in THF (1.56 L) was added to 1M LiAlH₄ solution in THF (777 mL, 0.777 mol) at about 0-10° C. over 1 h. After addition, the mixture was stirred for 2 h at 3° C. Water (27 mL) was added to the mixture dropwise to quench the reaction. 15% NaOH (27 mL) and water (80 mL) were sequentially added to the mixture dropwise. The mixture was stirred at r.t. for 1 h. DCM (2.35 L) was added to the mixture. The suspension was filtered through diatomaceous earth (100 g) bed and rinsed with DCM (300 mL). The filtrate was concentrated under reduced pressure and dried under vacuum oven at 40° C. for 18 h to give tert-butyl (1R,3R,5R)-3-(hydroxymethyl)-2-azabicyclo[3.1.0]hexane-2-carboxylate (133 g, 90% yield) as yellow oil which contained 2% of an isomer as determined by GCMS analysis. GCMS calc. for C₁₁H₁₉NO₃: 213.1. Found: 213.2 (M⁺). ¹H-NMR (400 MHz, CDCl₃) δ 4.83 (brs, 1H), 4.34 (brs, 1H), 2.45 (ddd, 1H), 1.55-1.43 (m, 12H), 0.80 (q, 1H), 0.40 (brs, 1H).

### Step 4. tert-Butyl (1R,3R,5R)-3-formyl-2-azabicyclo[3.1.0]hexane-2-carboxylate

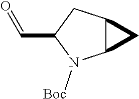

DMSO (42.7 mL, 0.603 mol) was added to oxalyl chloride (26.4 mL, 0.301 mol) in DCM (535 mL) dropwise at −78° C. over 30 min, while maintaining the temperature below −60° C. during addition. After stirring at −78° C. for 30 min, tert-butyl (1R,3R,5R)-3-(hydroxymethyl)-2-azabicyclo[3.1.0]hexane-2-carboxylate (53.5 g, 0.251 mol) in DCM (535 mL) was added to solution dropwise at −78° C. over 40 min. After stirring at −78° C. for 30 min, NEt₃ (104.9 mL, 0.753 mol) was added to solution dropwise at −78° C. over 40 min. After stirring at −78° C. for 1 h, the reaction mixture was warmed to 0° C. and stirred for 30 min. Water (888 mL) was added to the mixture and stirred for 20 min. The aqueous layer was separated and extracted with DCM (2×888 mL). The combined organic layers were sequentially washed with 1 M HCl (888 mL), water (888 mL) and saturated brine (888 mL). The organic layer was concentrated under reduced pressure to give tert-butyl (1R,3R,5R)-3-formyl-2-azabicyclo[3.1.0]hexane-2-carboxylate (44 g, 83% yield) as yellow oil. GCMS calc. for C₁₁H₇NO₃: 213.1. Found: 213.2 (M+). ¹H-NMR (400 MHz, CDCl₃) δ 9.54-9.31 (m, 1H), 4.64-4.39 (m, 1H), 3.68-3.45 (m, 1H), 2.68-2.33 (m, 1H), 2.24-2.10 (m, 1H), 1.53-1.41 (m, 10H), 0.88-0.71 (m, 1H), 0.39-0.28 (m, 1H).

### Step 5. tert-Butyl (1R,3R,5R)-3-ethynyl-2-azabicyclo[3.1.0]hexane-2-carboxylate

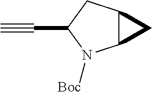

K₂CO₃ (28.8 g, 0.209 mol, 2 eq.) was added to a solution of tert-butyl (1R,3R,5R)-3-formyl-2-azabicyclo[3.1.0]hexane-2-carboxylate (22 g, 0.104 mol) in MeOH (352 mL) at 0-5° C. Dimethyl (1-diazo-2-oxopropyl)phosphonate (18.3 mL, 0.110 mol) was added to the mixture dropwise at 0-5° C. for 30 min while maintaining the temperature at <5° C. during addition. After stirring at 0-5° C. for 15 min., the reaction mixture was warmed up to r.t. and stirred for 2 h. Water (372 mL) and EtOAc (930 mL) was added to the mixture, which was stirred for 15 min. The aqueous layer was separated and extracted with EtOAc (372 mL). The combined organic layers were washed with water (560 mL) and brine (560 mL). The organic solution was concentrated under reduced pressure and purified via FCC eluted with a gradient of 0-10% EtOAc in heptane to give a 7 to 1 mixture of tert-butyl (1R,3R,5R)-3-ethynyl-2-azabicyclo[3.1.0]hexane-2-carboxylate and tert-butyl (1R,3S,5R)-3-ethynyl-2-azabicyclo[3.1.0]hexane-2-carboxylate (82 g, 74% calc. yield) as light yellow oil. GCMS calc. for C₁₂H₁₇NO₂: 207.1. Found: 207.0 (M+). ¹H-NMR (400 MHz, CDCl₃) δ 4.78-4.54 (m, 1H), 3.60-3.46 (m, 1H), 2.52-2.40 (m, 1H), 2.30-2.22 (m, 1H), 2.18-2.08 (m, 1H), 1.50-1.48 (m, 9H), 1.16-1.05 (m, 1H), 0.91-0.80 (m, 1H), 0.78-0.66 (m, 1H).

### Step 6. Cyclopropyl((1R,3R,5R)-3-ethynyl-2-azabicyclo[3.1.0]hexan-2-yl)methanone

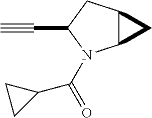

A mixture of tert-butyl (1R,3R,5R)-3-ethynyl-2-azabicyclo[3.1.0]hexane-2-carboxylate and tert-butyl (1R,3S,5R)-3-ethynyl-2-azabicyclo[3.1.0]hexane-2-carboxylate (82 g, 0.39 mol) and 4M HCl in dioxane (297 mL, 1.19 mol, 3 eq.) was stirred at rt for 4 h. The reaction mixture was diluted with THF (1.23 L) and cooled to 0° C. NEt₃ (275.8 mL, 1.98 mol) 30 was added to the reaction at 0° C. dropwise over 1.5 h while maintaining the temperature at <10° C. during addition. Cyclopropanecarbonyl chloride (45.4 g, 0.43 mol) was added to the reaction at 0° C. The reaction was warmed to r.t. and stirred for 3 h. 1M HCl (410 mL, 5 vol) and DCM (820 mL) was added. The aqueous layer was separated and extracted with DCM (2×820 mL). The combined organic layers were washed with water (820 mL) and brine (820 mL). The organic layer was concentrated under reduced pressure to give a crude residue (60 g). Diatomaceous earth (120 g) was added to the crude residue and the mixture was dried under reduced pressure to give a dried load powder (186 g). The dried load powder was purified via FCC eluted with a gradient of 15 to 40% EtOAc in heptane. The desired fractions were concentrated under reduced pressure and dried under vacuum at 30° C. for 18 h to give the title compound (40.8 g, 59% yield) as brown oil. GCMS calc. for C₁₂H₁₇NO₂: 175.1. Found: 175.0 (M+). ¹H-NMR (400 MHz, DMSOd₆) δ 5.14 (dt, 0.45H), 4.81 (dt, 0.55H), 3.82 (t, 0.55H), 3.71 (t, 0.45H), 3.42 (d, 0.45H), 3.15 (d, 0.55H), 2.57 (ddd, 0.45H), 2.44 (ddd, 0.55H), 2.09 (dd, 0.45H), 2.04 (ddd, 0.55H), 1.97 (dd, 0.55H), 1.86-1.69 (m, 1H), 1.62 (dddd, 0.45H), 1.01 (td, 0.55H), 0.90 (td, 0.45H), 0.87-0.68 (m, 5H).

### Example 4. Synthesis of tert-Butyl (1R,4R,5S)-5-amino-2-azabicyclo[2.1.1]hexane-2-carboxylate oxalate (step 14a in Example 1)

### Step 1. (E)-4-Methoxybut-3-en-2-one

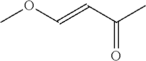

A mixture of 4,4-dimethoxy-2-butanone (350 g, 1.0 eq) and sodium acetate (11 g, 0.05 eq.) was heated to 145-150° C. under N₂ and the resulting MeOH is purged during the heating process. When the reaction is complete, the mixture was cooled to 70-80° C. The product was distilled under vacuum to give desired product (130 g, yield 50%). 1H NMR (CD₂Cl₂/CHDOD, 400 MHz): δ 7.60 (d, 1H, J=12.8 Hz) 5.53 (d, 1H, J=12.8 Hz), 3.81 (s, 3H,), 2.17 (s, 3H). 13C NMR (CD₂Cl₂/CD₃OD, 100.6 MHz): δ 27.1, 58.0, 107.0, 165.2, 199.6.

### Step 2. (E)-4-(Allylamino)but-3-en-2-one

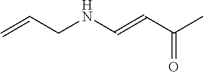

A mixture of (E)-4-methoxybut-3-en-2-one (150 g) and NEt₃ (182 g) in DCM (450 mL) was added was agitated under nitrogen at 10-15° C. Allylamine HCl (60%, 234 g) is slowly added to the mixture at 10-15° C. After the addition, the mixture is agitated for 30 min. When the reaction was completed, water (150 g) was added to the reaction mixture. The organic phase was separated, and the water phase was extracted with DCM (300 mL). The combined organic phases were washed with brine (150 mL) and organic phase was concentrated under vacuum to give crude product as yellow oil (175 g, yield 93%). 1H NMR (500 MHz, CDCl₃): δ 9.75 (bs, 1H); 6.58 (dd, 1H, J=16.8, 2); 5.78-5.86 (m, 1H); 5.19 (d, 1H, J=16.8)); 5.14 (d, 1H, J=10, 1)); 5.00 (d, 1H, J=10, 1); 3.74-3.77 (m, 2H); 2.03, (s, 3H). 13C NMR (125 Hz, CDCl3): 197.5; 153.2; 165.3; 117.6; 94.9; 51.1; 29.2.

### Step 3. tert-Butyl (E)-allyl(3-oxobut-1-en-1-yl)carbamate

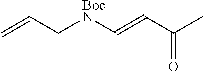 [SMILES: C=CCN(/C=C/C(C)=O)C(=O)OC(C)(C)C]

A mixture of (E)-4-(allylamino)but-3-en-2-one (130 g), trimethylamine (105 g), N,N-dimethylaminopyridine (13 g) in toluene (390 mL) was heated to 50-55° C. (Boc)₂O (259 g) was added in portion while maintained the reaction temperature between 50-55° C. After the reaction mixture was agitated for 2 h at 50-55° C. to complete the reaction. The mixture was cooled to 10-15° C. and 3 M aq. HCl was added to the mixture until the pH 5-6. The organic phase was separated, and the aqueous phase was extracted with toluene (260 mL). The combined organic phases were washed with water (260 mL). Activated charcoal (1 g) was added. The mixture was agitated at 50-55° C. for 1 h before cooling the mixture to 20-30° C. The mixture was filtered over diatomaceous earth bed and the diatomaceous earth bed was rinsed with toluene. The filtrated was concentrated to a residue and the residue was coevaporated with MeCN to give a residue as yellow oil (189 g, 80% yield). 1H NMR (500 MHz, CDCl₃): δ 8.11 (d, 1H, J=15); 5.68-5.73 (m, 1H); 5.49 (d, 1H, J=15); 5.14 (d, 1H, J=18); 5.09 (d, 1H, J=10); 4.13 (t, 2H); 2.20 (s, 3H); 1.50 (s, 9H). 13C NMR (125 MHz, CDCl₃): 198.6; 153.0; 143.2; 131.8; 117.8; 109.5; 84.0; 47.0; 28.3; 28.1.

### Step 4. tert-Butyl 5-acetyl-2-azabicyclo[2.1.1]hexane-2-carboxylate

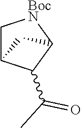

A solution of tert-butyl (E)-allyl(3-oxobut-1-en-1-yl)carbamate (270 g) in MeCN (3240 mL) was subjected to UV-photo reactor. When the reaction was complete, the yellow oil residue (major and minor isomer mixture) was used for next step without further purification. Sample was purified by column to get analytical data. 1H NMR (500 MHz, CDCl₃) δ 4.62-6.78 (bd, 1H); 3.40 (bt, 1H); 3.16 (bs, 1H); 3.06 (bs, 1H); 2.69 (s, 1H); 1.97 (s, 3H); 1.70-1.73 (m, 1H); 1.46 (s, 9H).

### Step 5. tert-Butyl (1R,4R,5S)-5-amino-2-azabicyclo[2.1.1]hexane-2-carboxylate oxalate

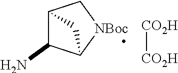 [SMILES: CC(C)(C)OC(=O)N1C[C@H]2C[C@@H]1[C@H]2N ; O=C(O)C(=O)O]

A mixture of tert-butyl 5-acetyl-2-azabicyclo[2.1.1]hexane-2-carboxylate (150 g) in MeCN (1500 mL) was added to sodium hypochlorite (173.5 g) in 30% NaOH (1.5 L) at 30-40° C. The mixture was agitated at 30-40° C. for 30 min. to complete the reaction. The mixture was cooled to 10-15° C. and 6M HCl aq. solution was added to adjust the mixture pH 8-9. The mixture was concentrated under vacuum to remove MeCN at 50-55° C. and MeOH (90 mL) was added to the residue. The mixture was cooled to 10-15° C. and 6M HCl was added to adjust the mixture pH 2-3 (solids precipitated out as the pH adjustment) and agitated for additional 2-3 h. The solids were isolated and rinsed with water (300 mL). The wet solids were dried under vacuum at 50-55° C.

Recrystallization: A mixture of the solids in toluene (1500 mL) was heated to 60-70° C. to a solution. (R)-(+)-1-phenylethylamine (80.7 g) was added at 40-70° C. The solution was cooled to 30-35° C. over 90 min (solids precipitated gradually) and agitated for 1 h. The suspension was cooled to 20-25° C. over 90 min and agitated for 2 h. The solids were isolated and rinsed with toluene (40 mL). A mixture of the cake and toluene (1200 mL) was heated to 100-105° C. to a solution. The mixture was cooled to 75-85° C. over 90 min. (solids precipitated) and agitated for 1 h. The mixture was cooled to 20-25° C. over 2 h and agitated for 2 h. The solids were isolated and rinsed with toluene (40 mL). The recrystallization process was repeated one more time.

Free base: to a mixture of the wet cake in toluene (225 mL) and water (225 mL) was added 30% aq. NaOH at 10-15° C. to pH 9-10. The mixture was agitated for 30 min and the organic phase was separated. To the aqueous phase was added 6 M aq. HCl at 10-15° C. to pH 2-3 (solids predicated). The mixture was then cooled to 3-8° C. and agitated for 1 h. The solids were isolated and washed with water (40 mL). The wet cake was dried under vacuum at 50-55° C. to give the desired (1R,4S,5S)-2-(tert-butoxycarbonyl)-2-azabicyclo[2.1.1]hexane-5-carboxylic acid (25 g, 18% yield).

A mixture of the acid (245 g), pyridine (86 g) and NH₄Cl (111 g) in MeCN (3700 mL) was added (Boc)₂O (310 g) at 15-25° C. The mixture was agitated for 5 h to complete the reaction. The solids were isolated and rinsed with MeCN (250 mL). The filtrate and rinse were combined and concentrated under vacuum at 40-45° C. and azeotroped with heptane. To the residue was added EtOAc (130 mL) and n-heptane (650 mL) at 40-45° C. The mixture was cooled to 10-15° C. (solids precipitated) and agitated for 2 h. The solids were isolated and rinsed with n-heptane (250 mL). The wet cake was dried under vacuum at 50-55° C. to give desired product tert-butyl (1R,4S,5S)-5-carbamoyl-2-azabicyclo[2.1.1]hexane-2-carboxylate quantitatively.

To cooled 15% aq. NaOH (800 mL) at 10-15° C. was added the tert-butyl (1R,4S,5S)-5-carbamoyl-2-azabicyclo[2.1.1]hexane-2-carboxylate (214 g). Sodium hypochlorite (91.2 g) was added at 10-20° C. and the mixture was agitated for 2 h. The mixture was heated to 40-45° C. for 4 h to complete the reaction. The reaction mixture was cooled to 15-20° C. and citric acid was added to adjust pH 5-6. The mixture was basified by addition of NaOH to pH 14. The basified mixture was extracted with 2-MelTHF (2×1000 mL). The combined organic phase was concentrated under vacuum and the residual was azeotroped with MeCN. The residue was dissolved in MeCN (140 mL) and activated charcoal (2 gram) was added. The mixture was agitated at 25-30° C. for 2 h. The mixture was filtered, and the filter bed is rinsed with MeCN (85 mL). The combined filtrate and rinse were added to a solution of oxalic acid (120 g) in MeCN (850 mL) at 40-45° C. The solution was cooled to 3-7° C. and agitated for 1 h. The solids were isolated and rinsed with MeCN (110 mL). The wet cake was dried at 40-50° C. under vacuum to give desired tert-butyl (1R,4R,5S)-5-amino-2-azabicyclo[2.1.1]hexane-2-carboxylate oxalate (248 g, 91% yield) as white solids. HPLC-MS for calc. C₁₀H₁₈N₂O₂: 198.14. Found (M+H): 199.1 ¹H NMR (500 MHz, DMSO-d₆): δ8.44 (s, 3H); 3.34, (m, 1H); 4.24, dt, 1H, J=6.9, 1.7 Hz); 3.20-3.31 (m, 2H); 2.84, (dt, 1H, J=6.5, 3.0); 1.65-1.71 (m, 1H); 1.42 (s, 9H); 1.19 (d, 1H, J=8.1). ¹³C NMR (125 Hz, DMSO-d₆): δ 165.0; 155.8; 79.5; 61.5; 50.6; 44.9; 40.8; 33.8; 28.6.

### Example 5: Synthesis procedure for Methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (Compound 2)

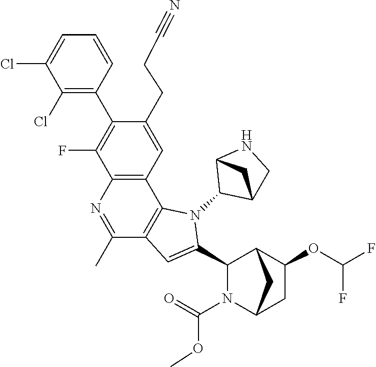 [SMILES: COC(=O)N1[C@@H]2C[C@@H]([C@@H](OC(F)F)C2)[C@@H]1c1cc2c(C)nc3c(F)c(-c4cccc(Cl)c4Cl)c(CCC#N)cc3c2n1[C@H]1[C@H]2CN[C@@H]1C2]

### Step 1. Methyl (1R,3R,4R,5S)-5-((tert-butyldiphenylsilyl)oxy)-3-ethynyl-2-azabicyclo[2.2.1]heptane-2-carboxylate

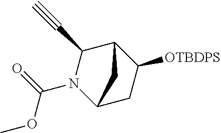

To a solution of Intermediate 5 (1.85 g, 3.89 mmol) in dioxane (10 mL) was added HCl (4 N in dioxane, 10 mL). The reaction was stirred at r.t. for 0.5 h. Upon completion, the volatiles were removed under reduced pressure. The residue was dissolved in DCM (20 mL). Upon stirring, N,N-diisopropylethylamine (2.0 mL, 11.7 mmol) was added followed by methyl chloroformate (0.6 mL, 7.78 mmol). The mixture was stirred for 0.5 h. Once completed, the reaction mixture was diluted with DCM, washed with water, dried over Na₂SO₄, filtered and concentrated. The crude product was purified by flash chromatography (0-50% EtOAc/hexanes) to afford the title compound. LC-MS calc. for C₂₆H₃₂NO₃Si (M+H)⁺: m/z=434.2. found 434.2.

### Step 2. Methyl (1R,3R,4R,5S)-3-ethynyl-5-hydroxy-2-azabicyclo[2.2.1]heptane-2-carboxylate

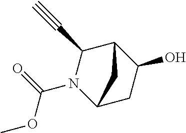

To a solution of methyl (1R,3R,4R,5S)-5-((tert-butyldiphenylsilyl)oxy)-3-ethynyl-2-azabicyclo[2.2.1]heptane-2-carboxylate (1.43 g, 3.30 mmol) in THF (11 mL) was added TBAF (1 N in THF, 4.0 mL, 3.96 mmol). The reaction was stirred at r.t. for 16 h. Upon completion, the volatiles were removed. The crude was purified by FCC (0-10% MeOH/DCM) to afford the title compound. LC-MS calc. for C₁₀H₁₄NO₃ (M+H)⁺: m/z=196.1; found 196.1.

### Step 3. Methyl (1R,3R,4R,5S)-5-(difluoromethoxy)-3-ethynyl-2-azabicyclo[2.2.1]heptane-2-carboxylate

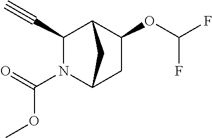 [SMILES: C#C[C@H]1[C@H]2C[C@H](C[C@@H]2OC(F)F)N1C(=O)OC]

To a flask containing methyl (1R,3R,4R,5S)-3-ethynyl-5-hydroxy-2-azabicyclo[2.2.1]heptane-2-carboxylate (0.514 g, 2.63 mmol) and copper(I) iodide (0.100 g, 0.527 mmol) was charged MeCN (13 mL). The mixture was stirred at 50° C. before an MeCN solution (2 mL) containing 2-(fluorosulfonyl)difluoroacetic acid (0.703 g, 3.95 mmol) was added slowly. The reaction mixture was stirred at 50° C. for 1 h. Upon completion, the mixture was concentrated under reduced pressure. The residue was dissolved in DCM, washed with saturated NaHCO₃ solution and water. The organic layer was dried over Na₂SO₄, filtered and concentrated. The crude product was purified by FCC (0-50% EtOAc/hexanes) to afford title compound (0.467 g, 72% yield). LC-MS calc. for C₁₁H₁₄F₂NO₃ (M+H)⁺: m/z=246.1. found 246.1.

### Step 4. tert-Butyl (1R,4R,5S)-5-((Rₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate

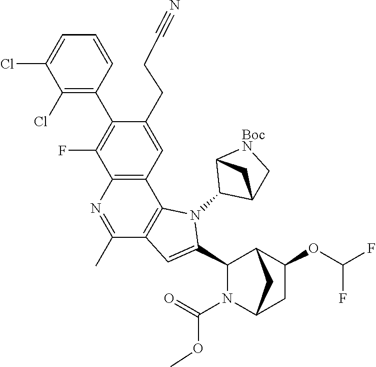 [SMILES: COC(=O)N1[C@@H]2C[C@@H]([C@@H](OC(F)F)C2)[C@@H]1c1cc2c(C)nc3c(F)c(-c4cccc(Cl)c4Cl)c(CCC#N)cc3c2n1[C@H]1[C@@H]2C[C@H]1N(C(=O)OC(C)(C)C)C2]

A mixture of Intermediate 2 (0.500 g, 0.734 mmol), methyl (1R,3R,4R,5S)-5-(difluoromethoxy)-3-ethynyl-2-azabicyclo[2.2.1]heptane-2-carboxylate (0.270 g, 1.10 mmol), copper(I) iodide (0.056 g, 0.294 mmol), tetrakis(triphenylphosphine)palladium(0) (0.170 g, 0.147 mmol) and N,N-diisopropylethylamine (1.3 mL, 7.34 mmol) in DMF (4.6 mL) was sparged with N₂ and heated at 70° C. for 1 h. Cs₂CO₃ (0.717 g, 2.20 mmol) was added to the reaction mixture. The resulting slurry was stirred at 90° C. for another 18 h. Upon completion, the mixture was cooled down to r.t. and poured into water. The solution was extracted with EtOAc twice. Then the combined organic layers were washed with brine five times, dried over Na₂SO₄, filtered and concentrated. The crude product was purified by FCC (0-100% EtOAc/hexanes) to afford the title compound. LC-MS calc. for C₄₀H₄₁Cl₂F₃N₅O₅(M+H)⁺: m/z=798.2. found 798.3.

### Step 5. Methyl (1R,3R,4R,5S)-3-((Rₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (Compound 2a)

To a solution of tert-butyl (1R,4R,5S)-5-((Rₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate (0.350 g, 0.439 mmol) in DCM (7 mL), was added MeCN (0.7 mL) and TFA (7 mL). The reaction was stirred at r.t. for 0.5 h. Upon completion, volatiles were removed under reduced pressure and the residue was dissolved in MeCN (4 mL) and water (1 mL) and purified by preparative LC-MS (XBridge® C18 column, eluting with a gradient of MeCN/water containing 0.1% TFA, at flow rate of 60 mL/min) to afford the title compound. LC-MS calc. for C₃₅H₃₃Cl₂F₃N₅O₃(M+H)⁺: m/z=698.2. found 698.2. ¹H NMR was collected on the TFA salt. ¹H NMR (500 MHz, DMSO-d₆) δ 9.53 (s, 1H), 8.24 (s, 1H), 8.19 (s, 1H), 7.86 (dd, J=8.1, 1.5 Hz, 1H), 7.59 (t, J=7.9 Hz, 1H), 7.47 (td, J=7.8, 1.6 Hz, 1H), 6.97 (s, 1H), 6.83 (t, J=75 Hz, 1H), 5.73 (s, 1H), 4.96 (m, 1H), 4.88 (m, 1H), 4.59 (s, 1H), 4.32 (s, 1H), 3.92 (m, 1H), 3.74 (s, 3H), 3.53 (m, 1H), 3.44 (m, 1H), 3.12-2.99 (m, 1H), 2.97-2.78 (m, 5H), 2.76-2.62 (m, 2H), 2.39-2.32 (m, 1H), 2.32-2.19 (m, 1H), 1.76-1.59 (m, 3H), 1.57-1.47 (m, 1H).

The alternative atropisomer methyl (1R,3R,4R,5S)-3-((Sₐ)-1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)-5-(difluoromethoxy)-2-azabicyclo[2.2.1]heptane-2-carboxylate (Compound 2b) can be prepared by an analogous route by performing processes analogous to the steps above starting from tert-butyl (1R,4R,5S)-5-((Sₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate instead of tert-butyl (1R,4R,5S)-5-((Rₐ)-8-(2-cyanoethyl)-7-(2,3-dichlorophenyl)-2-((1R,3R,4R,5S)-5-(difluoromethoxy)-2-(methoxycarbonyl)-2-azabicyclo[2.2.1]heptan-3-yl)-6-fluoro-4-methyl-1H-pyrrolo[3,2-c]quinolin-1-yl)-2-azabicyclo[2.1.1]hexane-2-carboxylate.

### Example 6: Antitumor Efficacy of the Combination of Compound 1 with Gemcitabine and Nab-Paclitaxel

### KPCY-013 Syngeneic Tumor Efficacy Model

Female C57BL/6 mice (Jackson Laboratory, aged 8-10 weeks) were inoculated subcutaneously with 2.5×10⁶ KPCY-013 (also identified as 2838c3 in Li, Byrne, Yan, et al.

Immunity (2018) 49:178-193) suspended in phosphate buffered saline. Mice were randomized into treatment groups and treatment of tumor-bearing mice started 8 days after tumor inoculation, when tumor volume reached approximately 320 mm³. KPCY-013 mice were randomized by tumor volume into groups of n=10. They were then administered monotherapy with either Compound 1 (30 mg/kg BID PO) or gemcitabine/nab-paclitaxel chemotherapy (gemcitabine 100 mg/kg Q7D IP+nab-paclitaxel 30 mg/kg Q7D IP) or Compound 1 in combination with gemcitabine/nab-paclitaxel, or vehicle control. Treatment was continuous throughout the study and ended on day 29 post-tumor implant. Mice were weighed and tumor measurements taken twice a week thru the end of the study. The tumor volume was calculated in 2 dimensions using the following equation:

$$volume = [ length * ( width 2 ) ] / 2.$$

Tumor growth inhibition (TGI) was calculated using the formula (1−[VT/VC])×100, where VT is the average tumor volume of the treatment group on the last day of treatment and VC is the average tumor volume of the control group on the last day of treatment. A partial response is defined as tumor volume ≤50% initial tumor volume for 2 consecutive measurements and a complete response is defined as tumor measuring ≤3 mm×3 mm (or ≤27 mm3) for 2 consecutive measurements. Statistical analyses were performed using GraphPad Prism software (v9.3.1; GraphPad Software, Boston, MA). Two-way ANOVA with Dunnet's multiple comparisons test was used to determine statistical differences between the treatment groups.

### Results

### Antitumor Activity of Compound 1±Gemcitabine/Nab-Paclitaxel Chemotherapy in the KPCY-013 Syngeneic Pancreatic Cancer Tumor Model

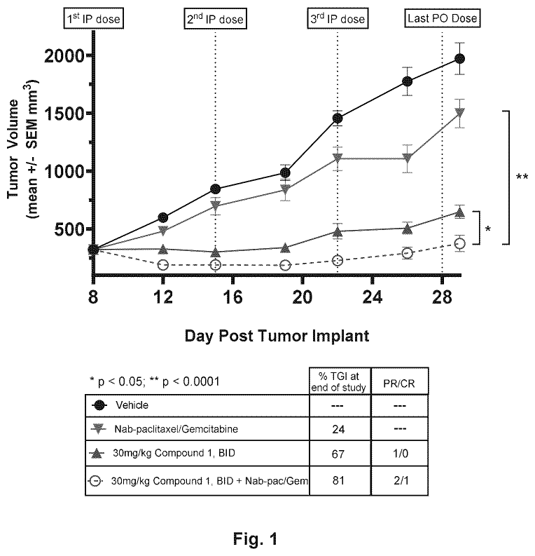

The antitumor activity of the combination of Compound 1±gemcitabine/nab-paclitaxel chemotherapy was evaluated in the KPCY-013 pancreatic cancer model. Mice were administered monotherapy with either Compound 1 (30 mg/kg BID PO) or gemcitabine/nab-paclitaxel chemotherapy (gemcitabine 100 mg/kg Q7D IP+nab-paclitaxel 30 mg/kg Q7D IP), both agents in combination, or vehicle control. When assessed on day 29, compared to the vehicle control, animals treated with Compound 1 (monotherapy or in combination) had significantly decreased tumor growth compared to the vehicle control (p s 0.006); gemcitabine/nab-paclitaxel did not deliver a significant decrease in tumor growth (FIG. 1). Combining Compound 1 with gemcitabine/nab-paclitaxel resulted in significantly decreased tumor growth compared to either monotherapy treatment with gemcitabine/nab-paclitaxel (p<0.0001) or monotherapy Compound 1 (p=0.0310). This demonstrates that combining Compound 1 with gemcitabine/nab-paclitaxel chemotherapy in pancreatic cancer preclinical models provides a therapeutic benefit in this setting.

### Example 7: HPAC Xenograft

Female NCr nude mice (Taconic Biosciences, aged 8-10 weeks) were inoculated subcutaneously with 5.5×10⁶ HPAC cells suspended in 50% phosphate buffered saline and 50% Matrigel®. Mice were randomized into treatment groups and treatment of tumor-bearing mice started 10 days after tumor inoculation, when tumor volume reached approximately 225 mm³. HPAC tumor bearing mice were randomized by tumor volume into groups of n=10. They were then administered monotherapy with either Compound 1 (30 mg/kg BID PO) or gemcitabine/nab-paclitaxel chemotherapy (gemcitabine 100 mg/kg Q7D IP+nab-paclitaxel 30 mg/kg Q7D IP) or Compound 1 in combination with gemcitabine/nab-paclitaxel, or vehicle control. Treatment was continuous throughout the study and ended on day 28 post-tumor implant. Mice were weighed and tumor measurements taken twice a week thru the end of the study. The tumor volume was calculated in 2 dimensions using the following equation:

$$volume = [ length * ( width 2 ) ] / 2.$$

Tumor growth inhibition (TGI) was calculated using the formula (1−[VT/VC])×100, where VT is the average tumor volume of the treatment group on the last day of treatment and VC is the average tumor volume of the control group on the last day of treatment.

### Results

### Antitumor Activity of Compound 1±Gemcitabine/Nab-Paclitaxel Chemotherapy in the HPAC Xenograft Pancreatic Cancer Tumor Model

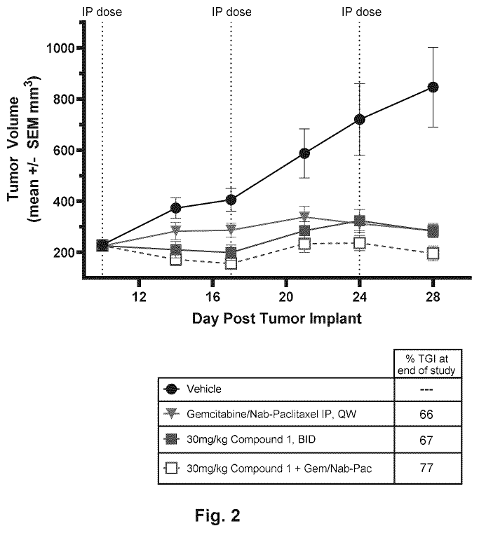

The antitumor activity of the combination of Compound 1±gemcitabine/nab-paclitaxel chemotherapy was evaluated in the HPAC pancreatic cancer model (FIG. 2). Mice were administered monotherapy with either Compound 1 (30 mg/kg BID PO) or gemcitabine/nab-paclitaxel chemotherapy (gemcitabine 100 mg/kg Q7D IP+nab-paclitaxel 30 mg/kg Q7D IP), both agents in combination, or vehicle control. When assessed on day 28, compared to the vehicle control animals treated with Compound 1 (monotherapy or in combination) exhibited 67% tumor growth inhibition while those treated with gemcitabine/nab-paclitaxel exhibited 66% growth inhibition, both in comparison to the vehicle control. Combining Compound 1 with gemcitabine/nab-paclitaxel resulted in tumor growth inhibition of 77%. This data supports the hypothesis that combining a KRAS G12D inhibitor such as Compound 1 with gemcitabine/nab-paclitaxel chemotherapy in pancreatic cancer preclinical models provides a therapeutic benefit compared to monotherapy.

### Example 8. Clinical Application

Subjects with select advanced or metastatic solid tumors are treated with Compound 1 given as monotherapy or in combination with GEMNabP, and the safety, tolerability, PK, pharmacodynamics, and clinical efficacy of the treatments are evaluated as described in this Example. Subjects treated in the present example are limited to those with documented G12D mutation. Treatment and evaluation includes 2 parts: monotherapy and combination with GEMNabP.

### Objectives and Endpoints

TABLE A

| | |
|---|---|
| Objectives | Endpoints |

Primary

| | |
|---|---|
| To demonstrate the safety and | Occurrence of DLTs |
| tolerability of Compound 1 as a single | Incidence of TEAEs |
| agent or in combination with GEMNabP. | Incidence of TEAEs leading to drug |
| | modifications (e.g., interruptions) and |
| | discontinuation of treatment. |

Secondary

| | |
|---|---|
| To evaluate the PK of Compound 1 as a | Plasma concentration of Compound 1 |
| single agent or in combination with | PK parameters for Compound 1 |
| GEMNabP. | including Cₘₐₓ, tₘₐₓ, C_{max ss}, C_{min ss}, |
| | AUC_{0-8 h}, AUC₀₋ₗₐₛₜ, AUC₀₋ₜₐᵤ, CL/F, |
| | V_{z}/F, and t½, as deemed appropriate. |
| To evaluate the effect of food on the PK | PK parameters for Compound 1 including |
| of Compound 1 | C_{max, ss}, tₘₐₓ, C_{min, ss}, and AUC₀₋ₜₐᵤ (Part 1d) |
| To determine the preliminary efficacy of | Objective response, defined as having a |
| Compound 1 as a single agent or in | best overall response of CR or PR (all |
| combination with GEMNabP. | subjects) per RECIST v1.1. |
| | Disease control, defined as having a best |
| | overall response of CR or PR, or SD per |
| | RECIST v1.1. |
| | DOR defined as the time from earliest date |
| | of disease response (CR or PR) until |
| | earliest date of disease progression by |
| | radiographic disease assessment per |
| | RECIST v1.1, or death due to any cause if |
| | occurring sooner than progression. |

Exploratory

| | |
|---|---|
| To demonstrate the additional | PFS, defined as the time from date of first |
| preliminary efficacy of Compound 1 as a | treatment to first progression (per RECIST |
| single agent. | v1.1) or death due to any cause if occurring |
| | sooner than progression (Part 1b Disease |
| | Groups 1, 2, and 3 only). |
| To demonstrate OS with Compound 1 as | OS defined as the time from date of first |
| a single agent. | treatment to death due to any cause (Part 1b |
| | Disease Groups 1, 2, and 3 only). |
| To demonstrate the pharmacodynamic | Measurement of molcular biomarkers (which |
| effects of Compound 1 as a single agent | may include genomic analysis of cfDNA/cfRNA |
| or in combination with GEMNabP in | and proteomic measurement of markers in |
| tumor tissue, blood, or plasma. | blood and genomic, transcriptomic, |
| | metabolomic, and/or proteomic markers in |
| | tumor tissue) that may indicate |
| | pharmacodynamic activity of Compound 1. |
| To identify molcular markers that can | Analysis of molcular biomarkers (which may |
| predict response or resistance to | include genomic analysis of cfDNA/cfRNA and |
| treatment with Compound 1 as a single | proteomic measurement of markers in blood |
| agent or in combination with GEMNabP | and genomic, transcriptomic, metabolomic, |
| in tumor tissue, blood, or plasma. | and/or proteomic markers in tumor tissue) that |
| | may be indicative of clinical response or |
| | clinical resistance (both primary and acquired) |
| | to Compound 1. |
| To evaluate alternative assays for the | Assessment of the performance of alternative |
| detection of KRAS G12D mutations in | diagnostic assays using tumor tissue biopsies |
| tumor tissue, blood, or plasma to identify | or liquid biopsies to detect KRAS G12D |
| the treatment population. | mutations. |
| To evaluate tumor metabolic response | Measurement of changes in tumor metabolic |
| (first 9 subjects in Part 1c only). | response compared to baseline using FDG |
| | PET. |

### Parts 1a and 2a: Dose Escalation

In Part 1a and Part 2a, dose escalation is performed using a statistical hybrid design to identify the MTD and/or ROE(s) based on the safety, tolerability, PK, pharmacodynamics, and preliminary clinical efficacy of Compound 1 when given as monotherapy or in combination with GEMNabP. Part 1a includes approximately 36 DLT-evaluable patients with any advanced or metastatic solid tumors treated with monotherapy. Part 2a includes approximately 18 DLT-evaluable subjects treated with Compound 1 in combination with GEMNabP (Combination Group 1). Combination Group 1 includes subjects with PDAC.

Part 1a treatment commences at 200 mg QD with the option to escalate or de-escalate based on defined DLT criteria. Part 2a combination treatment commences at doses of Compound 1 that are a minimum of 1 dose level below the highest tested dose of Compound 1 in Part 1a that is determined to be tolerable.

In Part 1a and Part 2a, cohorts of at least 3 DLT-evaluable subjects are initially selected for treatment. The first time a Compound 1 dose level is administered (monotherapy or combination), at least a 24-h gap is ensured after administration of Compound 1 to the first participant before subsequent participants begin treatment.

DLTs occurring up to and including cycle 1, day 28 (C1D28) guide dose escalation/de-escalation and determination of the RDE(s) and/or MTD. However, late-onset toxicities are considered when assessing safety through 30 days after the last dose of Compound 1 as monotherapy or in combination with GEMNabP. Therefore, treatment can include a lower dose of Compound 1. For example, a RDE may be determined based on relevant toxicities that become evident after Day 28 of treatment. While determination of the MTD will follow the definition of a DLT that occurs up to and including C1D28, the totality of the available data is considered when defining RDE(s).

Dose interruptions and/or modifications may be implemented based on toxicity. Dose escalation of Compound 1 can be permitted to a dose level that has been determined to be safe and tolerable. Additional subjects may be treated at any tolerable dose level to further characterize safety, PK, and/or pharmacodynamic biomarkers.

### Dose Levels of Compound 1

Initially, subjects are administered Compound 1 at a starting dose of 200 mg QD PO in continuous 28-day treatment cycles. Based on a first subject's treatment response, Compound 1 doses may be increased up to 2-fold in successive cohorts. If Grade 2 toxicity that has a reasonable possibility of being related to the treatment (not including toxicities with a clear alternative explanation or transient abnormal laboratory values without clinically significant signs or symptoms) is observed in 2 or more subjects at a dose level, then the subsequent dose increase is less than 2-fold. If the first dose level is not tolerable, the subject may be administered a reduced dose level. Dose de-escalations are performed as needed with at least 25% reductions of the dose and based on available unit dose (e.g., tablet) strengths. Dose escalation continues until the MTD is reached and/or the RDE(s) is (are) determined.

Subjects can receive a starting dose of Compound 1 at one of the following dose levels: 200 mg, 400 mg, 800 mg, 1200 mg, and 1600 mg. The dosing frequency (e.g., QD or BID dosing) is determined with consideration of safety, PK, and pharmacodynamic data for the individual subject and/or the subject population. For one or more subjects, the highest dose level administered is 1600 mg for QD dosing and 1200 mg for BID dosing.

### Hybrid Statistical Design to Guide Dose Escalation

For one or more subjects, treatment includes dose-escalation, dose de-escalation, and/or dose-elimination. A hybrid of the modified mTPI design and a dose-toxicity model is used to make dose-escalation, dose de-escalation, and dose-elimination decisions for Parts 1a and 2a. For each dosing regimen, dose-escalation decisions are based on a minimum cohort of 3 DLT-evaluable subjects and takes a target DLT rate of 28% during the first 28 days of treatment into consideration. However, even if an observed DLT rate supports dose escalation, a dosing regimen may be retained and the RDE(s) may be guided by PK and other clinical data (such as lower-grade treatment-related toxicities and AEs meeting DLT criteria that occurred after the DLT observation period).

The hybrid design (Liao et al., Int J Cancer, 2022, 1602-1610) is a hybrid of the modified mTPI design and a dose-toxicity model and consists of 3 steps.
Step 1: An mTPI design (Ji et al., Clin Trials, 2010, 7, 653-663) with a target DLT rate pT of 28% and a dose-exclusion cutoff of 0.95 is firstly modified to control the overdosing toxicity using the posterior probability of DLT rate in the overdosing interval (0.31, 1) to be less than 0.75. With this rule, if 3 DLTs are observed out of 6 participants, which is a DLT rate of about 50%, then the modified mTPI will guarantee a dose de-escalation when the observed toxicity rate is high. The dose-escalation rules based on the number of DLTs observed in a dose-level cohort is shown in Table 1.
Step 2: A frequentist logistic dose-toxicity model is used by pooling all observed safety information from all previous doses to estimate the DLT rate for the current dose level and predict the DLT rate for the next dose level in the provisional dose list. The 2-parameter logistic regression model of DLT rates against the dose levels can be written as log i t(pj)=α+β×dj, where α and β are 2 unknown parameters and pj is the DLT probability for dose level dj. The estimated DLT rate at the current dose level is used together with the decision rules from the modified mTPI in Table 1 to make a decision jointly about dose escalation. Note that if the dose-toxicity model is not feasible (e.g., no DLT is observed at any tested doses), then no action is needed at this step.
Step 3: If the decision in Table 1 is to have a dose escalation (E) to the next dose level in the provisional dose list, then the predicted DLT rate using the dose-toxicity model from Step 2 is used to judge whether the next dose level is feasible or not by checking if the predicted DLT rate at the next dose level is over the prespecified targeted DLT rate. If the predicted DLT rate is over the targeted DLT rate, then the next dose level in the provisional dose list cannot be used. Instead, an intermediate dose from the earlier used dose-toxicity model will be calibrated so that the DLT rate is below the targeted DLT rate. Similarly, if the decision in Table 1 is to have a dose de-escalation (D) to a lower dose level in the provisional dose list, an intermediate dose from the earlier used dose-toxicity model will be calibrated so that the DLT rate is below the targeted DLT rate. If the decision is to stay (S) at the current dose and the estimated DLT rate at the current dose is greater than the prespecified targeted DLT rate, then the decision is to de-escalate (D) to an intermediate dose level; otherwise, it is a stay (S). Note that choosing the intermediate dose level will take into consideration what is clinically and operationally feasible.

To demonstrate the safety and tolerability of Compound 1 as a single agent or in combination with GEMNabP, a minimum of 3 evaluable subjects are treated at each dose level. However, depending on the accrual rate and the minimal threshold of PK sampling needed, 3, 4, 5, or 6 subjects may be treated. Approximately 15 subjects total per dose level can be treated to further characterize safety, tolerability, PK, pharmacodynamics, and recommended combinatorial doses in preparation for further evaluation. Approximately 36 evaluable subjects are treated in the dose-finding part of the treatment, and the dose-escalation procedure is stopped if the number of evaluable subjects treated at any dose level is 9. If collected data support de-escalation of an unacceptable dose (D or DU) at the lowest dose level, a lower dose (or alternative schedule) is considered. When initiating treatment of a subject at a dose level in response to a “Stay (S)” decision or in case of revisiting the existing dose level, the number of additional subjects initiating treatment may be capped to minimize the exposure to a dose that may have unacceptable toxicity (denoted as “Dose Unacceptable (DU)” in Table 1). To determine how many more subjects can be enrolled at the dose level, a skilled artisan can count steps in a diagonal direction (down and to the right) from the current cell to the first cell marked DU. For example, if 1 of 3 subjects have experienced a DLT at a given dose level, no more than 3 additional subjects should initiate treatment at this dose level until additional DLT data are available. This is because that dose level would be considered unacceptably toxic if all 3 of the additional subjects experience a DLT (i.e., 4 of 6 subjects with a DLT in Table 1).

At the end of the dose-escalation procedure, the DLT rates at all tested dose levels are estimated based on the aforementioned dose-toxicity model if it is feasible or the pool adjacent violators algorithm if the parametric dose-toxicity model is not feasible. The dose with an estimated DLT rate closest to 28% is treated as an MTD. However, the totality of the available data, such as the collected safety, PK, and pharmacodynamic data, are considered before deciding on the dose(s) to carry forward to Parts 1b and 2b.

TABLE 1
Dose-Finding Rules per Modified mTPI Design

| Number of Participants | Number of Participants Evaluable for DLT | | | | | | |
|---|---|---|---|---|---|---|---|
| With at Least one DLT | 3 | 4 | 5 | 6 | 7 | 8 | 9 |
| 0 | E | E | E | E | E | E | E |
| 1 | S | S | S | S | E | E | E |
| 2 | D | D | S | S | S | S | S |
| 3 | DU | DU | D | D | D | S | S |
| 4 | | DU | DU | DU | DU | D | D |
| 5 | | | DU | DU | DU | DU | DU |
| 6 | | | | DU | DU | DU | DU |
| 7 | | | | | DU | DU | DU |
| 8 | | | | | | DU | DU |
| 9 | | | | | | | DU |

D = de-escalate to the next lower dose; DU = the current dose is unacceptably toxic; E = escalate to the next higher dose; S = stay at the current dose.
Note:
Target toxicity rate pT: 28%.
Note:
Flat noninformative prior Beta(1,1) is used as a prior and ε1 = ε2 = 0.03 (Ji et al. 2010, Ji and Wang, J Clin Oncol, 2013, 31, 1785-1791).
Note:
Posterior toxicity probability cut: 0.75.

### Parts 1b and 2b: Dose Expansion

Dose expansion is performed to further characterize the safety, tolerability, PK, pharmacodynamics, and preliminary antitumor activity of Compound 1 as monotherapy (Part 1b) or in combination with GEMNabP(Part 2b) administered at the selected RDEs based on Part 1a and Part 2a with the totality of available data. The number of subjects to be treated can be based on the ORR of a treatment cohort reaching a pre-determined threshold.

In Part 1b, for Disease Group 1 (PDAC), up to ≈20 subjects per RDE and up to ≈40 subjects in 1 selected RDE receive Compound 1 as monotherapy, the latter including at least 15 subjects who have received no more than 1 prior systemic regimen in the metastatic setting for PDAC. For Disease Groups 2 and 3 (CRC and NSCLC), approximately 20 subjects receive Compound 1 as monotherapy at each RDE. For Disease Group 4 (any other tumor-agnostic indication), up to ≈40 subjects receive Compound 1 as monotherapy at each RDE. If preliminary antitumor activity (defined as ORR ≥20%) is observed at a certain RDE in Disease Group 1, an additional 20 subjects may be included in this cohort. If preliminary antitumor activity (defined as ORR ≥15%) is observed at a certain RDE and tumor type in Disease Groups 2 or 3 in Part 1b, enrollment of these expansion cohorts can be increased to include up to approximately 40 patients each, as applicable. The totality of data collected, including responders as well as patients with SD, are considered in the decision to expand.

In Part 2b, up to ≈40 participants with PDAC administered Compound 1 in combination with GEMNabP per RDE are included in Combination Group 1. Multiple RDEs may be identified in Part 1a or 2a and any available data from a pharmacodynamic cohort. In the event that more than 1 RDE is used for Parts 1b and 2b, patients are randomized 1:1 to the RDE(s) within the selected group. On the basis of the totality of emerging data, an RDE cohort may be expanded or discontinued.

### Stopping Rules in Dose Expansion

The eligibility for treatment can be based on the likelihood that a potential subject will experience an AE during treatment. After treatment is initiated in Part 1b and Part 2b, further selection of subjects is suspended if at least 40% of 5 or more subjects meet the following criteria: In Part 1b or Part 2b Combination Group 1 has an AE Grade 4 during the first 3 cycles of treatment that is attributable to the drug.

### Overall Treatment Duration

A subject is considered to have completed treatment when all periods/parts of the treatment regimen is complete. For example, treatment is considered completed when the last subject's last visit has occurred. For treatment cohorts, such as a population of subjects defined by one more common features (e.g., a common disease), the end of treatment can be the date of the last visit of the final subject who began treatment, the date of receipt of the last datapoint from the last subject for primary or secondary analysis, or the last scheduled procedure shown for the last subject. Thus, the data collected are representative of all geographic regions.

A treatment regimen includes up to 28 days for screening, continuous treatment in consecutive 28-day cycles as long as subjects are receiving benefit and have not met any criteria for treatment discontinuation, and at least 30 days for safety follow-up. Subjects receiving combination therapy Antibody A also receive a 90-day safety follow-up. The duration of treatment for one or more subjects can be approximately 12 months.

After three or more subjects are treated for more than 6 months, the collected data can be analyzed. Subjects may continue to receive treatment per usual standard of care for this population (e.g., Q4W to assess for AEs/SAEs, safety laboratory assessments for treatment administration, and standard-of-care timing for radiology assessments for progression of disease or recurrence). The subject's clinician monitors for any AEs that may lead to drug discontinuation, SAEs, pregnancies, and deaths. The treatment continues until a discontinuation criterion is met.

### Subject Population

Subjects selected for treatment meet the following inclusion criteria:

### Inclusion Criteria

1. Ability to comprehend and willingness to sign a written ICF for treatment.
2. Age 18 years or older at the time of signing the ICF.
3. Willing and able to conform to and comply with all Protocol requirements, including all scheduled visits and Protocol procedures.
4. Life expectancy greater than 12 weeks.
5. ECOG performance status score of 0 or 1.
6. Locally advanced or metastatic solid tumor with KRAS G12D mutation.
   a. Subjects have a documented KRAS G12D alteration from tumor tissue or cfDNA as determined by local laboratories qualified per national country regulations using a PCR- or NGS-based assay approved for in vitro diagnostic use by local country regulations and used per the manufacturer's defined intended use, or alternatively for EU sites, an in-house IVD assay meeting the IVDR article 5(5) requirements.
7. For Part 1a and Part 2, Combination Group 1: Disease progression on prior standard treatment, intolerance to or ineligibility for standard treatment, declined available therapies that are known to confer clinical benefit, or no standard available treatment to improve the disease outcome. The number of prior standard treatments for specific subparts must not exceed those described in Inclusion Criterion 8.
8. Cohort-specific requirements as follows:
   a. Parts 1a and 1d: histologically or cytologically confirmed malignant solid tumor of any tissue origin
   b. Part 1b
      Disease Group 1: Diagnosis of PDAC and ≤2 prior systemic regimens in the metastatic setting.
         Note 1: Neoadjuvant or adjuvant therapy may be counted as one line of therapy if recurrence or development of metastatic disease occurred within 6 months of the last dose of therapy.
         Note 2: At least 15 subjects with no more than one prior systemic regimen in the metastatic setting are included.
      Disease Group 2: Diagnosis of CRC
      Disease Group 3: Diagnosis of NSCLC
      Disease Group 4: Diagnosis of other (not part of Disease Groups 1, 2, or 3) advanced solid tumor
   c. Part 1c:
      Confirmed diagnosis of CRC or
      Confirmed diagnosis of PDAC and <2 prior systemic regimens in the metastatic setting
   d. Parts 2a and 2b
      Combination Group 1 (Compound 1 in combination with GEMNabP):
      Part 2a:
         Diagnosis of PDAC and
         <1 prior systemic regimen in the metastatic setting
         Note 1: Neoadjuvant or adjuvant therapy will be counted as 1 line of therapy if recurrence or development of metastatic disease occurred within 6 months of the last dose of therapy.
         Note 2: Subjects who have received no prior systemic therapy for pancreatic cancer may have received up to 1 cycle of GEMNabP at the time of treatment commencement. The last chemotherapy dose must be at least 7 days before C1D1.
      Part 2b:
         Diagnosis of PDAC and
         No prior systemic regimen in the metastatic setting
         Note 1: Neoadjuvant or adjuvant therapy will be counted as 1 line of therapy if recurrence or development of metastatic disease occurred within 6 months of the last dose of therapy.
         Note 2: Subjects may have received up to 1 cycle of GEMNabP at the time of treatment commencement. The last chemotherapy dose must be at least 7 days before C1D1.
9. Measurable lesions by CT or MRI based on RECIST v1.1 criteria that are considered nonamenable to surgery or other curative treatments or procedures. Locally advanced disease must not be amenable to resection with curative intent or other curative treatments or procedures. Tumor lesions that are located in a previously irradiated area or in an area subjected to other locoregional therapy should only be selected as target lesions if progression has been demonstrated in such lesions.
10. Availability of a baseline archival tumor specimen or willingness to undergo a pretreatment biopsy (core or excisional; fine-needle aspirate samples are not acceptable) to obtain the specimen.
   a. Parts 1a, 1b, 1d, 2a, and 2b: fresh pretreatment biopsy (within the screening period) or archival tissue (collected within <24 months prior to C1D1).
   b. Part 1c: fresh pretreatment biopsy (within the screening period) or archival tissue (collected within <2 months prior to C1D1 or with medical monitor approval if a few days older) and willingness to undergo an on-treatment tumor biopsy (core or excisional; fine-needle aspirate samples are not acceptable).
      Note: Tumor lesions selected as target lesions should not be selected for biopsy.
11. Ability to swallow and retain oral medication.
12. Willingness to avoid pregnancy or fathering children based on the criteria below:
   a. Male participants with reproductive potential must agree to take appropriate precautions to avoid fathering children from screening through 180 days after the last dose of Compound 1 and GEMNabP and must refrain from donating sperm during this period. Permitted methods in preventing pregnancy should be communicated to the participants and their understanding confirmed.
   b. Female participants who are WOCBP must have a negative serum pregnancy test at screening and a negative urine pregnancy test before the first dose on Day 1 and must agree to take appropriate precautions to avoid pregnancy from screening through 180 days after the last dose of Compound 1 and GEMNabP and must refrain from donating oocytes during this period. Permitted methods in preventing pregnancy should be communicated to the participants and their understanding confirmed.
   c. Female participants not considered to be of childbearing potential are eligible.

### Exclusion Criteria

Subjects are excluded from treatment if any of the following criteria apply:

### Cancer History

1. Known additional invasive malignancy within 1 year of the first dose of Compound 1. Note: The following are allowed:
   Prior additional malignancy from which the participant has been disease-free for
   >1 year after treatment with curative intent.
   Noninvasive or indolent malignancy.
   Definitively treated early-stage basal cell or squamous cell carcinoma of the skin, superficial bladder cancer, in situ cervical cancer, early-stage endometrial cancer, Gleason 6/7 prostate cancer, ductal carcinoma in situ, or lobular carcinoma in situ of the breast.
2. Untreated brain or CNS metastases or brain/CNS metastases that have progressed (e.g., evidence of new or enlarging brain metastasis, new neurological symptoms attributable to brain or CNS metastases, new associated edema).
   Note: Participants (except those in Part 1c) with previously treated and clinically stable brain or CNS metastases are eligible if all of the following apply:
      There is no evidence of progression by imaging obtained after at least 2 weeks post-treatment.
      Any neurologic symptoms have returned to baseline.
      Use of corticosteroids for treatment of brain metastases has not been required for at least 7 days before the first dose of Compound 1.

### Prior Cancer Therapy

3. History of organ transplant, including allogeneic stem cell transplantation.
4. Treatment with anticancer therapies (including curative radiation to the thorax or systemic anticancer therapies) within 5 half-lives or 28 days (whichever is shorter) or participation in another interventional clinical study within 28 days before the first administration of Compound 1.
5. Any prior treatment with the following anticancer therapies:
   a. Parts 1a, 1c, 1d, and 2: Prior treatment with any KRAS G12D selective inhibitor. Part 1b: Prior treatment with any KRAS inhibitor.
6. Any prior radiation therapy within 28 days before the first dose of Compound 1.
Note: Palliative radiation therapy to single sites or small fields may be allowed but not within 1 week of the first dose of Compound 1.
Note: Participants must have recovered from all radiation-related toxicities, not require corticosteroids for this purpose, and not have had radiation pneumonitis.
7. Has not recovered to ≤Grade 1 from toxic effects of prior therapy and/or complications from prior surgical intervention before starting Compound 1.
Note: Participants with stable chronic conditions (s Grade 2) not expected to resolve (such as anemia not requiring transfusion support, neuropathy, hypothyroidism, and alopecia) are exceptions and may enroll.
8. Significant concurrent, uncontrolled medical condition, including but not limited to the following:
   a. Hepatic
Known history of DILI; alcoholic liver disease; nonalcoholic steatohepatitis; primary biliary cirrhosis; ongoing extrahepatic obstruction caused by stones, cirrhosis of the liver, or portal hypertension.
   b. Cardiovascular
History of clinically significant or uncontrolled cardiac disease, including recent (within the last 12 months) unstable angina pectoris or acute myocardial infarction, or New York Heart Association Class Ill or IV cardiac disease, including preexisting clinically significant ventricular arrhythmia, congestive heart failure, cardiomyopathy not controlled by medication, history of long QT syndrome, or other clinically significant heart disease (i.e., ≥uncontrolled Grade 3 hypertension). Subjects with a pacemaker and well-controlled rhythm for at least 1 month before the first dose of Compound 1 are allowed.
Part 1c: Subjects with new onset of deep vein thrombosis or pulmonary embolism or a history of thromboembolism while on anticoagulation therapy within 3 months prior to enrollment.
   c. Gastrointestinal
Significant gastrointestinal disorder that could interfere with absorption, metabolism, or excretion of Compound 1, including gastrectomy, partial gastrectomy, or presence of a venting gastric tube that may interfere with absorption of Compound 1
Recent (s 3 months) history or ongoing partial or complete bowel obstruction, unless corrected by surgery.
Any concomitant condition of the upper gastrointestinal tract that precludes administration of oral medications.
   d. Pulmonary (Only Participants in Parts 2a and 2b)
Evidence of interstitial lung disease or active, noninfectious pneumonitis.
History of interstitial lung disease.
9. History or presence of an ECG abnormality that is clinically meaningful.
Screening QTcF interval >450 milliseconds is excluded; in the event that a single QTc is >450 milliseconds, the subject may enroll if the average QTc for the 3 ECGs is <450 milliseconds.
10. Chronic or current active infectious disease requiring systemic antibiotic, antifungal, or antiviral treatment within 14 days before the first dose of Compound 1.
   Note: If a participant has a positive screening test result for SARS-CoV-2 infection, they should be excluded until test normalization and clinical recovery.
11. Active HBV. HBV DNA must be undetectable upon testing. Subjects with cleared prior HBV infection, defined as HBsAg negative, HBsAb positive, HBcAb positive, and undetectable HBV DNA, are eligible for treatment.

For subjects with cleared prior HBV infection:
HBV prophylaxis should be considered.
Note: Subjects with no prior history of HBV infection who have been vaccinated against HBV and who have a positive anti-HBs result as the only evidence of prior exposure may receive treatment.
12. Active HCV (testing must be performed at screening to determine eligibility), defined as follows: positive anti-HCV result and quantitative HCV RNA test result greater than the lower limits of detection of the assay (RNA test only required if anti-HCV positive, can be done as part of screening if available locally).
   Note: Anti-HCV-positive subjects who received and completed treatment for HCV that was intended to eradicate the virus may receive treatment if HCV RNA levels are undetectable at least 12 weeks after the last dose of therapy. Anti-HCV-positive participants with no available confirmatory negative HCV RNA test results are excluded.
13. Known HIV infection (positive for HIV 1/2 antibodies).

### Medications

14. Treatment with prohibited medication within 5 half-lives or 28 days (whichever is shorter) before the first dose of Compound 1.
15. Known hypersensitivity or severe reaction to any component of drug(s) or formulation components.

### Organ Function

16. Laboratory values at screening as defined in Table 2.

TABLE 2
Exclusionary Laboratory Values

| | |
|---|---|
| Laboratory Parameter | Exclusion Criterion |

Hematology

| | | |
|---|---|---|
| a | Platelets | For Parts 1a, 1c, 1d, 2a, and 2b, <100 × 10⁹/L (not influenced by |
| | | transfusion, i.e., last platelet transfusion should have occurred >7 |
| | | days from screening) |
| b | Hemoglobin | For Parts 1a, 1c, 1d, 2a, and 2b, <9 g/dL (not influenced by |
| | | transfusion, i.e., last transfusion should have occurred >14 days |
| | | from screening) |
| c | ANC | <1.0 × 10⁹/L for Parts 1 and 2 |

Hepatic

| | | |
|---|---|---|
| d | ALT | ≥2.5 × ULN or ≥5 × ULN for participants with liver metastases |
| e | AST | ≥2.5 × ULN or ≥5 × ULN for participants with liver metastases |
| f | Total bilirubin | ≥1.5 × ULN, unless conjugated bilirubin is <ULN (conjugated |
| | | bilirubin only needs to be tested if total bilirubin exceeds the ULN). If |
| | | there is no institutional ULN, then direct bilirubin must be <40% of |
| | | total bilirubin. |
| | | Note: In no case can the total bilirubin exceed 3 × ULN (except for |
| | | participants with Gilbert syndrome, total bilirubin may be >3.0 |
| | | mg/dL). |

Renal

| | | |
|---|---|---|
| h | Estimated creatinine | <60 mL/min as calculated by the Cockcroft-Gault formula or |
| | clearance | measured by 24-h urine collection. |

Coagulation

| | | |
|---|---|---|
| i | INR or PT | >1.5 × ULN, unless on therapeutic anticoagulants and stable (>3 |
| | | months under the same regimen). |
| j | aPTT or PTT | >1.5 × ULN, unless on therapeutic anticoagulants and stable (>3 |
| | | months under same regimen). |

### Other Exclusions

17. Any major surgery within 28 days before the first dose of Compound 1.
18. Women who are pregnant or breastfeeding or expecting to conceive or men who are expecting to father children within the projected duration of the treatment, starting with the screening visit through 180 days after the last dose of Compound 1 treatment.
19. Any condition that would interfere with full participation in the treatment, including administration of Compound 1 and attending required visits; pose a significant risk to the subject; or interfere with interpretation of data.

### Treatment

### Treatments Administered

Compound 1 is administered as a monotherapy or in combination with GEMNabP in subjects with select advanced or metastatic solid tumors. Eligible subjects receive Compound 1 administered PO.

Compound 1 is administered continuously during 28-day cycles until a new anticancer therapy is started, disease progression, unacceptable toxicity, death, withdrawal of consent, occurrence of any Compound 1 discontinuation criterion, or the end of treatment, whichever is earlier. The Compound 1 dose and schedule depends on the phase of treatment, as follows:
Dose escalation: dose is based on the subject's pre-determined starting dose level.
Dose expansion: dose is the ROE or one of the ROEs as identified in the dose-escalation phase of treatment.
Pharmacodynamic and food-effect cohorts: dose is based on the subject's pre-determined starting dose level. Compound 1 doses are those declared tolerable after analysis of collected data from Part 1a.

GEMNabP is administered as indicated in Table 3.

TABLE 3

| | Treatment 1 | Study Treatment 2 (GEMNabP) | |
|---|---|---|---|
| Treatment name | Compound 1 | Nab-paclitaxel | Gemcitabine |
| Mechanism of | KRAS G12D | Chemotherapy | |
| action | inhibitor | | |
| Dose | Oral | Liquid | Liquid |
| formulation | | | |
| Administration | Starting regimen of 200 | Subjects with no prior therapy: Days 1, 8, | |
| instructions | mg QD PO in 28-day | and 15 of each 28-day cycle | |
| | cycles. Subsequent dose | Subjects with 1 prior therapy: Days 1 and 15 | |
| | regimens are determined | of each 28-day cycle | |
| | during treatment. | Nab-paclitaxel 125 | Gemcitabine 1000 |
| | Subjects refrain from | mg/m² administered | mg/m² administered |
| | eating large meals and | as an IV infusion over | as an IV infusion |
| | drinking large volumes | 30-40 min. Nab- | over 30-40 min |
| | for at least 1 h before | paclitaxel infusions is | immediately after |
| | and after each dose; | initiated ≈30 min | nab-paclitaxel |
| | however, a light snack | after Compound 1 | infusion. |
| | and small amounts (sips) | administration | |
| | of water are allowed | (immediately to 6 h | |
| | during this period. | after Compound 1 is | |
| | Compound 1 is | acceptable). | |
| | administered with a glass | | |
| | of water. | | |
| Packaging and | Each bottle / vial will be labeled as required per country requirement | | |
| labeling | | | |
| Storage | Store at ambient conditions | Per product | Per product |
| | (15° C.-30° C. or 59° F.-86° F.). | label | label |

Dose-Limiting Toxicity and Determination of Maximum Tolerated Dose and/or Pharmacologically Active Dose

Analysis of safety events according to the occurrence of DLTs are used to evaluate dose levels and establish safe and tolerable doses up to and possibly including the MTD, if establishment of the MTD is achieved. The RDE(s) is determined by evaluation of all safety, PK, pharmacodynamic, and preliminary antitumor activity from the dose-escalation and related data from the pharmacodynamic cohort as applicable.

### Definition of a Dose-Limiting Toxicity

A DLT is defined as the occurrence of any of the toxicities in Table 9 except toxicities with a clear alternative explanation (e.g., due to disease progression) or transient (≤72 h) abnormal electrolyte values without associated clinically significant signs or symptoms. Dose-limiting toxicities are only those occurring up to and including Day 28 of the first drug cycle for participants in Part 1a and Part 2a. All DLTs are assessed for severity using CTCAE v5.0 criteria.

For Part 1a, subjects receiving at least 75% of planned doses of Compound 1 at the level assigned to that cohort and 2 doses of Antibody A at the assigned dose level during the 28-day DLT observation period are considered evaluable for DLT assessment.

For Part 2a, subjects receiving at least 75% of planned doses of Compound 1 at the level assigned to that cohort in addition to the following during the 28-day DLT observation period of the first drug combination cycle or have had a DLT during the first drug combination cycle to be considered evaluable for DLT assessment: at least 75% of planned doses of each component of GEMNabP.

Participants who do not meet these criteria may be replaced to obtain sufficient evaluable participants to assess dose levels using the hybrid design.

Individual subject dose reductions are made based on events observed at any time during treatment; however, for the purposes of dose cohort escalation/de-escalation, expanding a dose cohort, and determining the MTD of Compound 1, decisions are made based on events that are observed from the first day of drug administration through and including the final day of Cycle 1 (Day 28). A lower MTD is subsequently be determined based on relevant toxicities that become evident after Day 28.

TABLE 4
Definition of Dose-Limiting Toxicity

| Toxicity | Definition |
|---|---|
| Nonhematologic | Any liver function abnormality that meets the definition of Hy's law: 1) |
| | ALT or AST elevation ≥3 × ULN, concurrent with 2) total bilirubin ≥2 × |
| | ULN without initial findings of cholestasis (elevated serum ALP <2 × |
| | ULN), and 3) no other apparent possible causes of |
| | aminotransferase elevation and hyperbilirubinemia, including but not |
| | limited to viral hepatitis, pre-existing chronic or acute liver disease, or |
| | the administration of other drug(s) known to be hepatotoxic. |
| | Any ≥Grade 3 nonhematologic toxicity EXCEPT: |
| | An event clearly associated with the underlying disease, |
| | disease progression, a concomitant medication, or comorbidity. |
| | Asymptomatic changes in amylase, lipase, or lipid profile. |
| | Note: Asymptomatic changes in amylase and lipase must be |
| | confirmed by abdominal imaging to exclude pancreatitis. |
| | Singular or nonfasting asymptomatic elevations in blood |
| | glucose with respect to normal reference ranges. |
| | Grade 3 nausea, vomiting, and diarrhea adequately controlled |
| | with medical therapy within 72 h. |
| | Grade 3 fatigue <1 week. |
| | Grade 3 rash in the absence of desquamation, with no |
| | mucosal involvement, that does not require systemic |
| | corticosteroids and that resolves to Grade 1 or baseline in ≤14 |
| | days. |
| | Grade 3 infusion-related reactions where 1) hospitalization is |
| | solely conducted for postevent monitoring and no other criterion |
| | for Grade 3 per CTCAE grading is met and 2) discharge is |
| | within 48 h of admission. |
| Hematologic | Grade 3 thrombocytopenia with clinically significant bleeding |
| | (requires hospitalization, transfusion of blood products, or other |
| | urgent medical intervention). |
| | Grade 4 thrombocytopenia. |
| | Grade ≥3 febrile neutropenia (ANC <1.0 × 10⁹/L and a single |
| | temperature |
| | >101° F./38.3° C. or a sustained temperature ≥100.4° F./38.0° C. for |
| | more than 1 h). |
| | Grade 4 neutropenia that does not recover to ≤Grade 2 in ≤7 days |
| | after interrupting Compound 1. |
| | Grade 4 anemia not explained by underlying disease or some other |
| | concomitant disorder. |
| Immune- | Grade 3 irAEs that do not improve to ≤Grade 2 within <5 days |
| related toxicity | and to ≤Grade 1 or baseline within 14 days with appropriate |
| (for Part 2 | care or corticosteroid therapy (except as specified below). |
| Combination | Grade 4 irAEs. |
| Group 1) | Exceptions: |
| | Grade 3 or 4 endocrinopathy that is adequately |
| | controlled with hormone supplementation. |
| | Grade 3 rash in the absence of desquamation, with no mucosal |
| | involvement, that does not require systemic corticosteroids and |
| | that resolves to Grade 1 or baseline in ≤14 days. |
| General | Although the rules for adjudicating DLTs in the context of dose |
| | escalation are specified in this table, an AE not listed in this |
| | table may be considered a DLT based on the emerging safety |
| | profile. |
| | An event with a clear alternative explanation (e.g., due to disease |
| | progression) or transient (≤72 h) abnormal electrolyte values |
| | without associated clinically significant signs or symptoms are |
| | exceptions to the criteria in this table. |

MTD
The MTD is defined as the dose with an estimated DLT rate closest to the target DLT rate of 28%. Dose-limiting toxicity will be defined as the occurrence of any of the toxicities in this table occurring up to and including C1D28.

### Procedures for Cohort Review and Dose Escalation

For one or more subjects, the initial dose level in Part 1a (dose escalation) is Compound 1 200 mg QD PO, administered as continuous dosing over 28-day cycles. For one or more subjects, the initial dose level in Part 2a is selected after a minimum 2 dose levels according to Part 1a have been determined to be tolerable. To ensure safety of the combination treatment, the Compound 1 doses in Part 2a (including the starting dose) are at a minimum 1 dose level below the highest tested dose of Compound 1 according to Part 1a.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A method of treating cancer in a subject in need thereof comprising administering to the subject a therapeutically effective amount of a KRAS G12D inhibitor, or a pharmaceutically acceptable salt thereof, a nucleoside analog, or a pharmaceutically acceptable salt thereof, and a microtubule inhibitor, or pharmaceutically acceptable salt thereof.

2. The method of claim 1, wherein the nucleoside analog is selected from gemcitabine, cytarabine, azacytidine, cladribine, decitabine, fluorouracil, floxuridine, fludarabine, and nelarabine.

3-5. (canceled)

6. The method of claim 1, wherein the microtubule inhibitor is paclitaxel or docetaxel.

7. (canceled)

8. The method of claim 1, wherein the microtubule inhibitor is nanoparticle albumin-bound paclitaxel (nab-paclitaxel).

9. The method of claim 1, wherein the KRAS G12D inhibitor has an IC₅₀ of about 100 nM or lower and is selective for inhibiting G12D versus wild-type KRAS.

10. (canceled)

11. The method of claim 1, wherein the KRAS G12D inhibitor is a compound of Formula I: 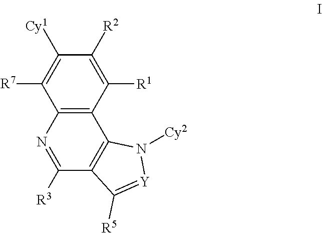
or a pharmaceutically acceptable salt thereof, wherein:
Y is N or CR⁶;
R¹ is selected from H, C₁₋₃ alkyl, C₁₋₃ haloalkyl, cyclopropyl, halo, D, CN, and OR^{a1}; wherein said C₁₋₃ alkyl and cyclopropyl are each optionally substituted with 1 or 2 substituents independently selected from R^{g};
R² is selected from H, C₁₋₃ alkyl, C₁₋₃ haloalkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene, halo, D, CN, and OR^{a2}; wherein said C₁₋₃ alkyl, 4-6 membered heterocycloalkyl, phenyl, 5-6 membered heteroaryl, 4-6 membered heterocycloalkyl-C₁₋₃ alkylene, phenyl-C₁₋₃ alkylene, 5-6 membered heteroaryl-C₁₋₃ alkylene are each optionally substituted with 1 or 2 substituents independently selected from R^{g};
Cy¹ is selected from C₃₋₁₀ cycloalkyl, 4-10 membered heterocycloalkyl, C₆₋₁₀ aryl and 6-10 membered heteroaryl; wherein the 4-10 membered heterocycloalkyl and 6-10 membered heteroaryl each has at least one ring-forming carbon atom and 1, 2, 3, or 4 ring-forming heteroatoms independently selected from N, O, and S; wherein a ring-forming carbon atom of 6-10 membered heteroaryl and 4-10 membered heterocycloalkyl is optionally substituted by oxo to form a carbonyl group; and wherein the C₃₋₁₀ cycloalkyl, 4-10 membered heterocycloalkyl, C₆₋₁₀ aryl and 6-10 membered heteroaryl are each optionally substituted with 1, 2, 3, or 4 substituents independently selected from R¹⁰;
R³ is selected from H, C₁₋₃ alkyl, C₁₋₃ haloalkyl, C₃₋₆ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR^{f3}, C(O)NR^{c3}R^{d3}, NR^{c3}R^{j3}, and NR^{c3}C(O)R^{b3}; wherein said C₁₋₃ alkyl, C₃₋₆cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1, 2, or 3 substituents independently selected from R³⁰;
R⁵ is selected from H, C₁₋₃ alkyl, C₁₋₃ haloalkyl, cyclopropyl, halo, D, CN, and OR^{a5}; wherein said C₁₋₃ alkyl and cyclopropyl are each optionally substituted with 1 or 2 substituents independently selected from R^{g};
R⁶ is selected from H, C₁₋₃ alkyl, C₁₋₃ haloalkyl, C₃₋₆ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, D, CN, OR^{a6}, and C(O)NR^{c6}R^{d6}; wherein said C₁₋₃ alkyl, C₃₋₆cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene are each optionally substituted with 1 or 2 substituents independently selected from R⁶⁰;
R⁷ is selected from H, C₁₋₃ alkyl, C₁₋₃ haloalkyl, cyclopropyl, halo, D, CN, and OR^{a7}; wherein said C₁₋₃ alkyl and cyclopropyl are each optionally substituted with 1 or 2 substituents independently selected from R^{g};
Cy² is selected from 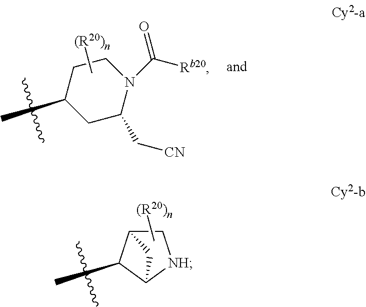
wherein n is 0, 1, or 2;
each R¹⁰ is independently selected from C₁₋₃ alkyl, C₁₋₃ haloalkyl, halo, D, CN, OR^{a10}, C(O)R^{b10}, C(O)NR^{c10}R^{d10}, C(O)OR^{a10}, NR^{c10}R^{d10}, and S(O)₂R^{b10};
each R²⁰ is independently selected from C₁₋₃ alkyl, C₁₋₃ haloalkyl, halo, D, CN, and OR^{a20};
each R³⁰ is independently selected from C₁₋₃ alkyl, C₁₋₃ haloalkyl, C₃₋₆ cycloalkyl, 4-6 membered heterocycloalkyl, phenyl, 5-6 membered heteroaryl, halo, D, CN, OR^{a30}, C(O)R^{b30}, C(O)NR^{c30}R^{d30}, C(O)OR^{a30}, NR^{c30}R^{d30}, and S(O)₂R^{b30}; wherein said C₁₋₃ alkyl, C₃₋₆cycloalkyl, 4-6 membered heterocycloalkyl, phenyl, and 5-6 membered heteroaryl are each optionally substituted with 1 or 2 substituents independently selected from R³¹;
each R³¹ is independently selected from C₁₋₃ alkyl, C₁₋₃ haloalkyl, halo, D, CN, OR^{a31}, C(O)R^{b31}, C(O)NR^{c31}R^{d31}, C(O)OR^{a31}, NR^{c31}R^{d31}, and S(O)₂R^{b31};
each R³³ is independently selected from C₁₋₃ alkyl, C₁₋₃ haloalkyl, C₃₋₆ cycloalkyl, 4-membered heterocycloalkyl, 6-membered heterocycloalkyl, phenyl, 5-6 membered heteroaryl, halo, D, CN, OR^{a30}, C(O)NR^{c30}R^{d30}, and NR^{c30}R^{d30}; wherein said C₁₋₃ alkyl, C₃₋₆cycloalkyl, 4-membered heterocycloalkyl, 6-membered heterocycloalkyl, phenyl, and 5-6 membered heteroaryl are each optionally substituted with 1 or 2 substituents independently selected from R³¹;
each R⁶⁰ is independently selected from C₁₋₃ alkyl, C₁₋₃ haloalkyl, 4-6 membered heterocycloalkyl, 5-6 membered heteroaryl, halo, D, CN, OR^{a60}, C(O)R^{b60}, C(O)NR^{c6}R^{d60}, NR^{c60}C(O)R^{b60}, C(O)OR^{a60}, NR^{c60}C(O)OR^{a60}, NR^{c60}R^{d60}, NR^{c60}S(O)₂R^{b60}, and S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, C₁₋₃ haloalkyl, halo, D, CN, OR^{a61}, and NR^{c61}R^{d61};
R^{a1} is selected from H, C₁₋₃ alkyl, and C₁₋₃ haloalkyl;
each R^{a2} is independently selected from H, C₁₋₃ alkyl, and C₁₋₃ haloalkyl;
each R^{b3}, R^{c3} and R^{d3} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R³⁰;
or R^{c3} and R^{d3} attached to the same N atom, together with the N atom to which they are attached, form a 4-, 5-, or 6-membered heterocycloalkyl group optionally substituted with 1, 2, or 3 substituents independently selected from R³⁰;
R^{j3} is selected from C₁₋₃ alkyl, C₁₋₃ haloalkyl, C₃₋₆ cycloalkyl, 4-6 membered heterocycloalkyl, phenyl, and 5-6 membered heteroaryl; wherein said C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R³⁰;
or R^{c3} and R^{j3} attached to the same N atom, together with the N atom to which they are attached, form a 4-, 5-, or 6-membered heterocycloalkyl group optionally substituted with 1, 2, or 3 substituents independently selected from R³⁰;
R^{f3} is selected from C₁₋₃ haloalkyl, C₃₋₆ cycloalkyl, 4-6 membered heterocycloalkyl, phenyl and 5-6 membered heteroaryl; wherein said C₁₋₃ haloalkyl, C₃₋₆ cycloalkyl, 4-6 membered heterocycloalkyl, phenyl, and 5-6 membered heteroaryl are each optionally substituted with 1, 2, or 3 substituents independently selected from R³⁰; or
R^{f3} is selected from 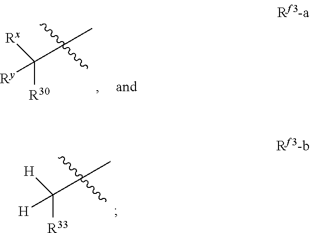
wherein R^{x} is H or C₁₋₂ alkyl and R^{y} is C₁₋₂ alkyl;
or R^{x} and R^{y}, together with the C atom to which they are attached, form a 3-, or 4-membered cycloalkyl group;
R^{a5} is selected from H, C₁₋₃ alkyl, and C₁₋₃ haloalkyl;
each R^{a6}, R^{c6} and R^{d6} is independently selected from H, C₁₋₃ alkyl, C₁₋₃ haloalkyl, C₃₋₆ cycloalkyl, 4-6 membered heterocycloalkyl, phenyl and 5-6 membered heteroaryl; wherein said C₁₋₃ alkyl, C₃₋₆ cycloalkyl, 4-6 membered heterocycloalkyl, phenyl and 5-6 membered heteroaryl are each optionally substituted with 1 or 2 substituents independently selected from R⁶⁰;
R^{a7} is selected from H, C₁₋₃ alkyl, and C₁₋₃ haloalkyl;
each R^{a10}, R^{b10}, R^{c10} and R^{d10} is independently selected from H, C₁₋₃ alkyl, and C₁₋₃ haloalkyl;
each R^{a20} is independently selected from H, C₁₋₃ alkyl, and C₁₋₃ haloalkyl;
R^{b20} is selected from NH₂, C₁₋₃ alkyl, and C₁₋₃ haloalkyl;
each R^{a30}, R^{b30}, R^{c30} and R^{d30} is independently selected from H, C₁₋₃ alkyl, and C₁₋₃ haloalkyl;
each R^{a31}, R^{b31}, R^{c31} and R^{d31} is independently selected from H, C₁₋₃ alkyl, and C₁₋₃ haloalkyl;
each R^{a60}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆ cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹;
each R^{a61}, R^{c61}, and R^{d61}, is independently selected from H, C₁₋₃ alkyl, and C₁₋₃ haloalkyl; and
each R^{g} is independently selected from D, OH, CN, halo, C₁₋₃ alkyl, C₁₋₃ haloalkyl, C₁₋₃ alkoxy, C₁₋₃ haloalkoxy, amino, C₁₋₃ alkylamino, and di(C₁₋₃ alkyl)amino.

12-13. (canceled)

14. The method of claim 11, wherein
Y is CR⁶;
R¹ is H;
R² is —CH₂CH₂CN;
Cy¹ is phenyl; wherein the phenyl is optionally substituted with 1 or 2 substituents independently selected from R¹⁰;
R³ is selected from H, C₁₋₃ alkyl, phenyl, and 5-6 membered heteroaryl; wherein said C₁₋₃ alkyl, phenyl, and 5-6 membered heteroaryl are each optionally substituted with 1, 2 or 3 substituents independently selected from R³⁰;
R⁵ is selected from H and halo;
R⁶ is selected from 4-8 membered heterocycloalkyl; wherein said 4-8 membered heterocycloalkyl is optionally substituted with 1 or 2 substituents independently selected from R⁶⁰; or
R⁶ is selected from C₁₋₃ alkyl; wherein said C₁₋₃ alkyl is substituted with 1 or 2 substituents independently selected from R⁶⁰;
R⁷ is halo;
Cy² is 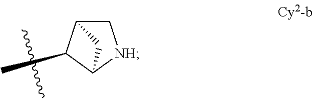
each R¹⁰ is independently selected from C₁₋₃ alkyl and halo;
each R³⁰ is independently selected from C₁₋₃ alkyl, halo, D, OH, and C(O)NR^{c30}R^{d30};
wherein said C₁₋₃ alkyl is optionally substituted with 1 substituent independently selected from R³¹;
each R³¹ is OR^{a31};
each R⁶⁰ is independently selected from C₁₋₃ alkyl, C₁₋₃ haloalkoxy, 4-6 membered heterocycloalkyl, 5-6 membered heteroaryl, halo, C(O)R^{b60}, C(O)NR^{c60}R^{d60}, NR^{c60}C(O)R^{d60}, C(O)OR^{a60}, NR^{c60}C(O)OR^{a60}, and NR^{c60}S(O)₂R^{b60}; wherein said C₁₋₃ alkyl, 4-6 membered heterocycloalkyl, and 5-6 membered heteroaryl are each optionally substituted with 1 or 2 substituents independently selected from R⁶¹;
each R⁶¹ is independently selected from C₁₋₃ alkyl, and halo;
each R^{c30} and R^{d30} is independently selected from H and C₁₋₃ alkyl;
each R^{a31} is independently selected from H and C₁₋₃ alkyl; and
each R^{a60}, R^{b60}, R^{c60} and R^{d60} is independently selected from H, C₁₋₃ alkyl, C₁₋₃ haloalkyl, C₃₋₆ cycloalkyl, 4-6 membered heterocycloalkyl, and 5-6 membered heteroaryl; wherein said C₁₋₃ alkyl, C₃₋₆ cycloalkyl, 4-6 membered heterocycloalkyl, and 5-6 membered heteroaryl are each optionally substituted with 1 or 2 substituents independently selected from R⁶¹;
or any R^{c60} and R^{d60} attached to the same N atom, together with the N atom to which they are attached, form a 4-, 5-, or 6-membered heterocycloalkyl group optionally substituted with 1 or 2 substituents independently selected from R⁶¹.

15-16. (canceled)

17. The method of claim 11, wherein the compound of Formula I is a compound of Formula III: 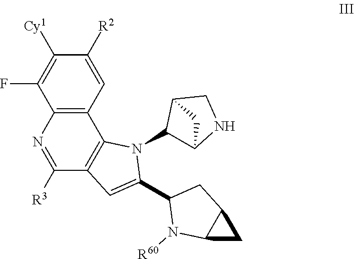
or a pharmaceutically acceptable salt thereof.

18. The method of claim 1, wherein the KRAS G12D inhibitor is selected from
3-(1-((1R,4R,5S)-2-azabicyclo[2.1.1]hexan-5-yl)-7-(7-chloro-3-hydroxynaphthalen-1-yl)-6-fluoro-2-methyl-4-(1H-1,2,4-triazol-1-yl)-1H-pyrrolo[3,2-c]quinolin-8-yl)propanenitrile;
3-(1-((1R,4R,5S)-2-azabicyclo[2.1.1]hexan-5-yl)-7-(5,7-difluoro-1H-indol-3-yl)-6-fluoro-2-methyl-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-6-fluoro-7-(6-fluoro-5-methyl-1H-indol-3-yl)-2-methyl-4-((S)-1-((S)-1-methylpyrrolidin-2-yl)ethoxy)-1H-pyrrolo[3,2-c]quinolin-8-yl)propanenitrile;
3-(2-(3-(azetidin-1-yl)-3-oxopropyl)-1-((1R,4R,5S)-2-azabicyclo[2.1.1]hexan-5-yl)-7-(2,3-dichlorophenyl)-6-fluoro-4-((S)-1-((S)-1-methylpyrrolidin-2-yl)ethoxy)-1H-pyrrolo[3,2-c]quinolin-8-yl)propanenitrile;
3-((1-((1R,4R,5S)-2-azabicyclo[2.1.1]hexan-5-yl)-6-fluoro-7-(3-hydroxynaphthalen-1-yl)-8-methyl-4-((S)-1-((S)-1-methylpyrrolidin-2-yl)ethoxy)-1H-pyrrolo[3,2-c]quinolin-2-yl)methyl)oxazolidin-2-one;
8-(1-((1R,4R,5S)-2-azabicyclo[2.1.1]hexan-5-yl)-6-fluoro-2,8-dimethyl-4-((S)-1-((S)-1-methylpyrrolidin-2-yl)ethoxy)-1H-pyrrolo[3,2-c]quinolin-7-yl)-1-naphthonitrile;
1-((2S,4S)-1-acetyl-2-(cyanomethyl)piperidin-4-yl)-7-(8-cyanonaphthalen-1-yl)-6-fluoro-4-((S)-1-((S)-1-methylpyrrolidin-2-yl)ethoxy)-1H-pyrazolo[4,3-c]quinoline-8-carbonitrile;
8-(1-((1R,4R,5S)-2-azabicyclo[2.1.1]hexan-5-yl)-6-fluoro-8-methyl-4-((S)-1-((S)-1-methylpyrrolidin-2-yl)ethoxy)-2-((3-oxomorpholino)methyl)-1H-pyrrolo[3,2-c]quinolin-7-yl)-1-naphthonitrile;
3-(7-(benzo[b]thiophen-3-yl)-1-((1R,4R,5S)-2-azabicyclo[2.1.1]hexan-5-yl)-6-fluoro-4-((S)-1-((S)-1-methylpyrrolidin-2-yl)ethoxy)-2-((2-oxopyrrolidin-1-yl)methyl)-1H-pyrrolo[3,2-c]quinolin-8-yl)propanenitrile;
3-(1-((1R,4R,5S)-2-azabicyclo[2.1.1]hexan-5-yl)-4-(((S)-1-(dimethylamino)propan-2-yl)oxy)-6-fluoro-7-(7-fluoronaphthalen-1-yl)-2-((2-oxopyrrolidin-1-yl)methyl)-1H-pyrrolo[3,2-c]quinolin-8-yl)propanenitrile;
8-(1-((1R,4R,5S)-2-azabicyclo[2.1.1]hexan-5-yl)-8-(2-cyanoethyl)-6-fluoro-2-methyl-4-((S)-1-((S)-1-methylpyrrolidin-2-yl)ethoxy)-1H-pyrrolo[3,2-c]quinolin-7-yl)-1-naphthonitrile;
3-(1-((1R,4R,5S)-2-azabicyclo[2.1.1]hexan-5-yl)-7-(2,3-dichloro-5-hydroxyphenyl)-6-fluoro-2-methyl-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-8-(2-cyanoethyl)-6-fluoro-4-((3-fluoro-1-methylazetidin-3-yl)methoxy)-7-(3-hydroxynaphthalen-1-yl)-1H-pyrrolo[3,2-c]quinolin-2-yl)-N,N-dimethylpropanamide;
3-(1-((1R,4R,5S)-2-azabicyclo[2.1.1]hexan-5-yl)-6-fluoro-7-(3-hydroxynaphthalen-1-yl)-2-methyl-4-(5-methylpyrazin-2-yl)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoronaphthalen-1-yl)-4-methyl-2-((4-methyl-2-oxopiperazin-1-yl)methyl)-1H-pyrrolo[3,2-c]quinolin-8-yl)propanenitrile;
3-(1-((1R,4R,5S)-2-azabicyclo[2.1.1]hexan-5-yl)-7-(2,3-dichloro-5-hydroxyphenyl)-4-ethoxy-6-fluoro-2-((4-isopropyl-2-oxopiperazin-1-yl)methyl)-1H-pyrrolo[3,2-c]quinolin-8-yl)propanenitrile;
3-(1-((1R,4R,5S)-2-azabicyclo[2.1.1]hexan-5-yl)-4-(3-(dimethylamino)-3-methylazetidin-1-yl)-6-fluoro-7-(7-fluoronaphthalen-1-yl)-2-((3-oxomorpholino)methyl)-1H-pyrrolo[3,2-c]quinolin-8-yl)propanenitrile;
3-(1-((1R,4R,5S)-2-azabicyclo[2.1.1]hexan-5-yl)-4-ethoxy-6-fluoro-7-(3-hydroxynaphthalen-1-yl)-2-(1-(3-oxomorpholino)ethyl)-1H-pyrrolo[3,2-c]quinolin-8-yl)propanenitrile;
3-(1-((endo)-2-azabicyclo[2.1.1]hexan-5-yl)-6-fluoro-7-(3-hydroxynaphthalen-1-yl)-4-((S)-1-((S)-1-methylpyrrolidin-2-yl)ethoxy)-2-(pyridin-3-yl)-1H-pyrrolo[3,2-c]quinolin-8-yl)propanenitrile;
3-(2-(3-(azetidin-1-yl)-3-oxopropyl)-1-((1R,4R,5S)-2-azabicyclo[2.1.1]hexan-5-yl)-7-(7,8-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;
3-(2-(3-(azetidin-1-yl)-3-oxopropyl)-1-((1R,4R,5S)-2-azabicyclo[2.1.1]hexan-5-yl)-7-(6,7-difluoronaphthalen-1-yl)-6-fluoro-4-((S)-1-((S)-1-methylpyrrolidin-2-yl)ethoxy)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoro-3-hydroxynaphthalen-1-yl)-2-methyl-4-((S)-1-((S)-1-methylpyrrolidin-2-yl)ethoxy)-1H-pyrrolo[3,2-c]quinolin-8-yl)propanenitrile;
1-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrazolo[4,3-c]quinolin-7-yl)isoquinoline-8-carbonitrile;
8-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrrolo[3,2-c]quinolin-7-yl)-1-naphthonitrile;
8-(1-((2S,4S)-1-acetyl-2-(cyanomethyl)piperidin-4-yl)-8-chloro-6-fluoro-4-((S)-1-((S)-1-methylpyrrolidin-2-yl)ethoxy)-1H-pyrazolo[4,3-c]quinolin-7-yl)-1-naphthonitrile;
3-(1-((1R,4R,5S)-2-azabicyclo[2.1.1]hexan-5-yl)-6-fluoro-7-(7-fluoro-3-hydroxynaphthalen-1-yl)-2-methyl-4-(1H-1,2,4-triazol-1-yl)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoronaphthalen-1-yl)-2-methyl-4-((S)-1-((S)-1-methylpyrrolidin-2-yl)ethoxy)-1H-pyrrolo[3,2-c]quinolin-8-yl)propanenitrile;
(2R)-2-(1-((1R,4R,5S)-2-azabicyclo[2.1.1]hexan-5-yl)-8-(2-cyanoethyl)-7-(2,3-dichlorophenyl)-6-fluoro-4-(1H-1,2,4-triazol-1-yl)-1H-pyrrolo[3,2-c]quinolin-2-yl)-N,N-dimethylpyrrolidine-1-carboxamide;
methyl (2R)-2-(1-((1R,4R,5S)-2-azabicyclo[2.1.1]hexan-5-yl)-7-(2-chloro-3-methylphenyl)-8-(2-cyanoethyl)-6-fluoro-4-(1H-1,2,4-triazol-1-yl)-1H-pyrrolo[3,2-c]quinolin-2-yl)pyrrolidine-1-carboxylate;
methyl (1S,3R,5S)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;
3-(1-((1R,4R,5S)-2-azabicyclo[2.1.1]hexan-5-yl)-7-(2,3-dichlorophenyl)-6-fluoro-4-methyl-2-(5-oxo-1,2,3,5-tetrahydroindolizin-3-yl)-1H-pyrrolo[3,2-c]quinolin-8-yl)propanenitrile;
methyl (2R)-2-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)pyrrolidine-1-carboxylate;
methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(methylcarbamoyl)pyridin-3-yl)-1H-pyrrolo[3,2-c]quinolin-2-yl)pyrrolidine-1-carboxylate;
3-(1-((1R,4R,5S)-2-azabicyclo[2.1.1]hexan-5-yl)-7-(2-chloro-3-fluorophenyl)-2-((R)-1-(cyclopropanecarbonyl)pyrrolidin-2-yl)-6-fluoro-4-methyl-1H-pyrrolo[3,2-c]quinolin-8-yl)propanenitrile;
8-(2-((R)-1-acetylpyrrolidin-2-yl)-1-((1R,4R,5S)-2-azabicyclo[2.1.1]hexan-5-yl)-6-fluoro-8-methyl-4-(2-methylpyridin-4-yl)-1H-pyrrolo[3,2-c]quinolin-7-yl)-1,2,3,4-tetrahydronaphthalene-1-carbonitrile;
5-(1-((1R,4R,5S)-2-azabicyclo[2.1.1]hexan-5-yl)-7-(3-chloro-2-methylphenyl)-8-(2-cyanoethyl)-6-fluoro-2-((R)-1-(2-oxopyrazin-1(2H)-yl)ethyl)-1H-pyrrolo[3,2-c]quinolin-4-yl)-N-methylpicolinamide;
3-(1-((1R,4R,5S)-2-azabicyclo[2.1.1]hexan-5-yl)-6-fluoro-7-(7-fluoronaphthalen-1-yl)-4-(6-(2-hydroxypropan-2-yl)pyridin-3-yl)-2-((R)-1-(2-oxopyrazin-1(2H)-yl)ethyl)-1H-pyrrolo[3,2-c]quinolin-8-yl)propanenitrile;
3-(1-((1R,4R,5S)-2-azabicyclo[2.1.1]hexan-5-yl)-7-(3-chloro-2-methylphenyl)-6-fluoro-4-(5-methylpyrazin-2-yl)-2-((R)-1-(2-oxopyrazin-1(2H)-yl)ethyl)-1H-pyrrolo[3,2-c]quinolin-8-yl)propanenitrile;
methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(5-fluoro-6-(methylcarbamoyl)pyridin-3-yl)-1H-pyrrolo[3,2-c]quinolin-2-yl)pyrrolidine-1-carboxylate;
methyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
ethyl (2R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
3-(1-((1R,4R,5S)-2-azabicyclo[2.1.1]hexan-5-yl)-7-(2,3-dichlorophenyl)-2-((R)-1-(3,3-difluoroazetidine-1-carbonyl)pyrrolidin-2-yl)-6-fluoro-4-(methyl-d3)-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-2-((R)-1-(3,3-difluoroazetidine-1-carbonyl)pyrrolidin-2-yl)-6-fluoro-1H-pyrrolo[3,2-c]quinolin-8-yl)propanenitrile;
3-(1-((1R,4R,5S)-2-azabicyclo[2.1.1]hexan-5-yl)-7-(3-chloro-2-methylphenyl)-6-fluoro-4-(5-methylpyrazin-2-yl)-2-((R)-1-(3-oxomorpholino)ethyl)-1H-pyrrolo[3,2-c]quinolin-8-yl)propanenitrile;
5-(1-((1R,4R,5S)-2-azabicyclo[2.1.1]hexan-5-yl)-8-(2-cyanoethyl)-6-fluoro-7-(7-fluoronaphthalen-1-yl)-2-((R)-1-(3-oxomorpholino)ethyl)-1H-pyrrolo[3,2-c]quinolin-4-yl)-N-methylpicolinamide;
3-(1-((1R,4R,5S)-2-azabicyclo[2.1.1]hexan-5-yl)-6-fluoro-7-(7-fluoronaphthalen-1-yl)-4-(6-(2-hydroxypropan-2-yl)pyridin-3-yl)-2-((R)-1-(3-oxomorpholino)ethyl)-1H-pyrrolo[3,2-c]quinolin-8-yl)propanenitrile;
3-(1-((1R,4R,5S)-2-azabicyclo[2.1.1]hexan-5-yl)-6-fluoro-7-(7-fluoronaphthalen-1-yl)-4-(5-methylpyrazin-2-yl)-2-((R)-1-(3-oxomorpholino)ethyl)-1H-pyrrolo[3,2-c]quinolin-8-yl)propanenitrile;
methyl (1R,3R,5R)-3-(1-((1R,4R,5S)-2-azabicyclo[2.1.1]hexan-5-yl)-8-(2-cyanoethyl)-7-(2,3-dichlorophenyl)-4-(6-(dimethylcarbamoyl)pyridin-3-yl)-6-fluoro-1H-pyrrolo[3,2-c]quinolin-2-yl)-2-azabicyclo[3.1.0]hexane-2-carboxylate;
3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-6-fluoro-4-(6-(2-hydroxypropan-2-yl)pyridin-3-yl)-2-((R)-1-(2-oxopyrazin-1(2H)-yl)ethyl)-1H-pyrrolo[3,2-c]quinolin-8-yl)propanenitrile;
methyl (2R,4S)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-4-fluoropyrrolidine-1-carboxylate;
methyl (2R,5R)-2-(1-((1R,4R,5S)-2-azabicyclo[2.1.1]hexan-5-yl)-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)-5-methylpyrrolidine-1-carboxylate;
methyl (2R)-2-(1-((1R,4R,5S)-2-azabicyclo[2.1.1]hexan-5-yl)-3-chloro-8-(2-cyanoethyl)-7-(2,3-dichlorophenyl)-6-fluoro-4-(6-(2-hydroxypropan-2-yl)pyridin-3-yl)-1H-pyrrolo[3,2-c]quinolin-2-yl)pyrrolidine-1-carboxylate;
4-(1-((1R,4R,5S)-2-azabicyclo[2.1.1]hexan-5-yl)-8-(2-cyanoethyl)-7-(2,3-dichlorophenyl)-6-fluoro-2-((R)-1-(2-oxopyrazin-1(2H)-yl)ethyl)-1H-pyrrolo[3,2-c]quinolin-4-yl)-2-fluoro-N-methylbenzamide;
methyl ((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)carbamate;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-2,2-difluoroacetamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-2,2-difluoroacetamide;
(2S)—N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)TH F-2-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)cyclopropanesulfonamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)thiazole-4-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-N-methylcyclopropanecarboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-methylcyclopropane-1-carboxamide;
3-(1-((1R,4R,5S)-2-azabicyclo[2.1.1]hexan-5-yl)-7-(2,3-dichlorophenyl)-6-fluoro-4-(1-hydroxyethyl)-2-((1R,3R,5R)-2-(1-methylcyclopropane-1-carbonyl)-2-azabicyclo[3.1.0]hexan-3-yl)-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-(1-hydroxyethyl)-1H-pyrrolo[3,2-c]quinolin-8-yl)propanenitrile;
3-(1-((1R,4R,5S)-2-azabicyclo[2.1.1]hexan-5-yl)-7-(2,3-dichlorophenyl)-6-fluoro-2-((1R,3R,5R)-2-(1-fluorocyclopropane-1-carbonyl)-2-azabicyclo[3.1.0]hexan-3-yl)-4-methyl-1H-pyrrolo[3,2-c]quinolin-8-yl)propanenitrile;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclopropane-1-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-(1-hydroxyethyl)-1H-pyrrolo[3,2-c]quinolin-2-yl)ethyl)-1-fluorocyclobutane-1-carboxamide;
3-(1-((1R,4R,5S)-2-azabicyclo[2.1.1]hexan-5-yl)-7-(3-chloro-2-methylphenyl)-2-(1-(2,6-dimethyl-3-oxo-2,3-dihydropyridazin-4-yl)ethyl)-6-fluoro-4-methyl-1H-pyrrolo[3,2-c]quinolin-8-yl)propanenitrile;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)pyrimidine-4-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)pyridazine-3-carboxamide;
N-((1R)-1-(1-((1R,4R,5S)-2-azabicyclo[2.1.1]hexan-5-yl)-8-(2-cyanoethyl)-7-(2,3-dichlorophenyl)-6-fluoro-4-methyl-1H-pyrrolo[3,2-c]quinolin-2-yl)ethyl)-3,3-difluoroazetidine-1-carboxamide;
3-(1-((1R,4R,5S)-2-azabicyclo[2.1.1]hexan-5-yl)-7-(2,3-dichlorophenyl)-6-fluoro-4-methyl-2-((R)-1-((1-methyl-1H-pyrazol-4-yl)amino)ethyl)-1H-pyrrolo[3,2-c]quinolin-8-yl)propanenitrile;
5-(1-((1R,4R,5S)-2-azabicyclo[2.1.1]hexan-5-yl)-8-(2-cyanoethyl)-7-(2,3-dichlorophenyl)-6-fluoro-2-((R)-1-(1-fluorocyclopropane-1-carbonyl)pyrrolidin-2-yl)-1H-pyrrolo[3,2-c]quinolin-4-yl)-N,N-dimethylpicolinamide; and
methyl (2R)-2-(1-((1R,4R,5S)-2-azabicyclo[2.1.1]hexan-5-yl)-8-(2-cyanoethyl)-7-(2,3-dichlorophenyl)-4-(4-((dimethylamino)methyl)-2,3-difluorophenyl)-6-fluoro-1H-pyrrolo[3,2-c]quinolin-2-yl)pyrrolidine-1-carboxylate;
and pharmaceutically acceptable salts thereof.

19. The method of claim 1, wherein the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

20. The method of claim 1, wherein the KRAS G12D inhibitor is 3-((Rₐ)-1-((1R,4R,5S)-2-azabicyclo [2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

21. The method of claim 1, wherein the KRAS G12D inhibitor is 3-((Rₐ)-1-((1R,4R,5S)-2-azabicyclo [2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride.

22. The method of claim 1, wherein the KRAS G12D inhibitor is 3-((Rₐ)-1-((1R,4R,5S)-2-azabicyclo [2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate.

23. The method of claim 1, wherein the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride.

24. The method of claim 1, wherein the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate.

25-28. (canceled)

29. A method of treating cancer in a subject in need thereof comprising administering to the subject a KRAS G12D inhibitor that is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, a nucleoside analog that is gemcitabine, or a pharmaceutically acceptable salt thereof, and a microtubule inhibitor that is nab-paclitaxel, or a pharmaceutically acceptable salt thereof.

30. The method of claim 29, wherein the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

31. The method of claim 29, wherein the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride.

32. The method of claim 29, wherein the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate.

33. (canceled)

34. The method of claim 1, to 3 wherein
the KRAS G12D inhibitor is administered twice daily (BID) or once daily (QD), and is administered orally (PO);
the nucleoside analog is administered once weekly and is administered as an intraperitoneal injection (IP); and
the microtubule inhibitor is administered once weekly and is administered as an intraperitoneal injection (IP).

35-40. (canceled)

41. The method of claim 1, wherein the cancer is selected from a carcinoma, a hematological cancer, a sarcoma, and glioblastoma.

42-43. (canceled)

44. The method of claim 1, wherein the cancer is a hematological cancer selected from myeloproliferative neoplasms, myelodysplastic syndrome, chronic and juvenile myelomonocytic leukemia, acute myeloid leukemia, acute lymphocytic leukemia, and multiple myeloma.

45. The method of claim 1, wherein the cancer is a carcinoma selected from pancreatic, colorectal, lung, bladder, gastric, esophageal, breast, head and neck, cervical, skin, and thyroid carcinomas.

46-49. (canceled)

50. The method of claim 1, wherein the cancer is non-small cell lung cancer (NSCLC) or pancreatic ductal adenocarcinoma.

51. (canceled)

52. The method of claim 1, wherein the cancer is metastatic.

53-61. (canceled)

62. A pharmaceutical combination comprising a KRAS G12D inhibitor, or a pharmaceutically acceptable salt thereof, a nucleoside analog, or a pharmaceutically acceptable salt thereof, and a microtubule inhibitor, or a pharmaceutically acceptable salt thereof.

63. The pharmaceutical combination of claim 62, wherein the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

64. The pharmaceutical combination of claim 62, wherein the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof.

65. The pharmaceutical combination of claim 62, wherein the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride.

66. The pharmaceutical combination of claim 62, wherein the KRAS G12D inhibitor is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate.

67. The pharmaceutical combination of claim 62, wherein the KRAS G12D inhibitor is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate.

68. The pharmaceutical combination of claim 62, wherein the nucleoside analog is gemcitabine, or a pharmaceutically acceptable salt thereof, and the microtubule inhibitor is nab-paclitaxel.

69-72. (canceled)

73. The method of claim 1 comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, gemcitabine, or a pharmaceutically acceptable salt thereof, and a microtubule inhibitor, or pharmaceutically acceptable salt thereof.

74. The method of claim 1 comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride, gemcitabine, or a pharmaceutically acceptable salt thereof, and a microtubule inhibitor, or pharmaceutically acceptable salt thereof.

75. The method of claim 1 comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate, gemcitabine, or a pharmaceutically acceptable salt thereof, and a microtubule inhibitor, or pharmaceutically acceptable salt thereof.

76. The method of claim 1 comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, a nucleoside analog, or a pharmaceutically acceptable salt thereof, and nab-paclitaxel.

77. The method of claim 1 comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride, a nucleoside analog, or a pharmaceutically acceptable salt thereof, and nab-paclitaxel.

78. The method of claim 1 comprising administering to the subject 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate, a nucleoside analog, or a pharmaceutically acceptable salt thereof, and nab-paclitaxel.

79-81. (canceled)
